(19) United States
(12) Patent Application Publication
(10) Pub. No.: US 2014/0357512 A1
(43) Pub. Date: Dec. 4, 2014

(54) HISTONE DEACETYLASE (HDAC) BIOMARKERS IN MULTIPLE MYELOMA

(71) Applicant: ACETYLON PHARMACEUTICALS, INC., Boston, MA (US)

(72) Inventors: Min Yang, Boston, MA (US); Simon S. Jones, Boston, MA (US)

(73) Assignee: ACETYLON PHARMACEUTICALS, INC., Boston, MA (US)

(21) Appl. No.: 14/294,833

(22) Filed: Jun. 3, 2014

Related U.S. Application Data
(60) Provisional application No. 61/830,371, filed on Jun. 3, 2013.

(51) Int. Cl. G01N 33/574; G01N 33/50

(52) U.S. Cl. CPC G01N 33/57407; G01N 33/57496; G01N 33/5011; G01N 2333/98; G01N 2500/02
USPC 506/9; 530/328; 530/326; 530/327; 530/324; 435/7.1

(57) ABSTRACT

The invention relates to histone deacetylase (HDAC) biomarkers in multiple myeloma. Specifically, the biomarkers are drug specific, histone deacetylase (HDAC) or HDAC6 biomarker peptides, which are acetylated, for multiple myeloma. Alternatively, the biomarkers are drug specific, HDAC6 biomarker peptides, which are acetylated or unacetylated, for multiple myeloma. The invention also relates to a kit comprising a detection agent and instructions for identifying a biomarker peptide of the invention. The invention further relates to a method for monitoring treatment efficiency of an HDAC inhibitor in a subject.

### CROSS REFERENCE TO RELATED APPLICATIONS

This application claims priority to U.S. Provisional Application Ser. No. 61/830,371, filed Jun. 3, 2013, which is hereby incorporated by reference in its entirety.

### FIELD OF THE INVENTION

Provided herein are histone deacetylase (HDAC) biomarkers in multiple myeloma. Specifically, the biomarkers are drug specific, histone deacetylase (HDAC) or HDAC6 biomarker peptides, which are acetylated, for multiple myeloma. Alternatively, the biomarkers are drug specific, HDAC6 biomarker peptides, which are acetylated or unacetylated, for multiple myeloma. The invention also relates to a kit comprising a detection agent and instructions for identifying a biomarker peptide of the invention. The invention further relates to a method for monitoring treatment efficiency of an HDAC inhibitor in a subject.

### BACKGROUND OF THE INVENTION

Cancer is one of the leading causes of death in the United States and in the world. It is estimated that approximately 1.6 million new cases of cancer will occur in the United States in 2012. It is also estimated that approximately 575,000 people will die from cancer in the United States in 2012.

Cancer grows out of normal cells in the body. Normal cells multiply when the body needs them, and die when the body doesn't need them. Cancer occurs when the cells in the body grow and multiply out of control.

There are many causes of cancer, such as exposure to carcinogenic chemicals, use of tobacco, drinking excess alcohol, exposure to environmental toxins, exposure to excessive sunlight, genetic problems, obesity, radiation, and viruses. In addition, the cause of many cancers remains unknown.

There are many different types of cancer, which can develop in almost any organ or tissue in the body. One type of cancer is multiple myeloma.

Multiple myeloma, also known as plasma cell myeloma or Kahler's disease, is a cancer of plasma cells. In multiple myeloma, collections of abnormal plasma cells accumulate in the bone marrow, where they interfere with the production of normal blood cells.

Because many organs can be affected by myeloma, the symptoms and signs vary greatly. Effects of myeloma include elevated calcium, renal failure, anemia, and bone lesions.

Myeloma is generally thought to be treatable, but incurable. Remission may be induced with steroids, chemotherapy, proteasome inhibitors, immunomodulatory drugs such as thalidomide or lenalidomide, and stem cell transplants.

Myeloma develops in 1-4 per 100,000 people per year. With conventional treatment, median survival is 3-4 years, which may be extended to 5-7 years or longer with advanced treatments. Multiple myeloma is the second most common hematological malignancy in the U.S. (after non-Hodgkin lymphoma), and constitutes 1% of all cancers.

Accordingly, there is a need to quickly and reliably identify biomarkers that are indicative of treatment efficiency in multiple myeloma.

### SUMMARY OF THE INVENTION

To meet this and other needs, provided herein are histone deacetylase (HDAC) biomarkers in multiple myeloma and methods of using such biomarkers. Specifically, the biomarkers are drug specific, histone deacetylase (HDAC) or HDAC6 biomarker peptides, which are acetylated, for multiple myeloma. Alternatively, the biomarkers are drug specific, HDAC6 biomarker peptides, which are acetylated or unacetylated, for multiple myeloma.

An embodiment of the invention comprises a Compound B specific histone deacetylase 6 (HDAC6) biomarker peptide, which is acetylated, for multiple myeloma comprising (or consisting of) an amino acid sequence selected from the group consisting of SEQ ID NOs: 1-18, and 323-490.

Another embodiment of the invention comprises a Tubastatin A specific histone deacetylase 6 (HDAC6) biomarker peptide, which is acetylated, for multiple myeloma comprising (or consisting of) an amino acid sequence selected from the group consisting of SEQ ID NOs: 1-322.

An additional embodiment of the invention comprises a joint Compound B and Tubastatin A specific histone deacetylase 6 (HDAC6) biomarker peptide, which is acetylated, for multiple myeloma comprising (or consisting of) an amino acid sequence selected from the group consisting of SEQ ID NOs: 1-18.

Another embodiment of the invention comprises a Compound A specific histone deacetylase (HDAC) biomarker peptide, which is acetylated, for multiple myeloma comprising (or consisting of) an amino acid sequence selected from the group consisting of SEQ ID NOs: 12-18, 245-322, and 476-817.

An additional embodiment of the invention comprises a Compound B specific histone deacetylase 6 (HDAC6) biomarker peptide, which is acetylated or unacetylated, for multiple myeloma comprising (or consisting of) an amino acid sequence selected from the group consisting of SEQ ID NOs: 818-15721.

A further embodiment of the invention comprises a kit comprising an anti-acetylated lysine antibody and instructions for identifying an acetylated biomarker peptide comprising (or consisting of) an amino acid sequence selected from the group consisting of SEQ ID Nos: 1-817.

Alternatively, an embodiment of the invention comprises a kit comprising a detection agent and instructions for identifying an acetylated or unacetylated biomarker peptide comprising (or consisting of) an amino acid sequence selected from the group consisting of SEQ ID NOs: 818-15721.

A further embodiment of the invention comprises a method for monitoring the treatment efficiency of a histone deacetylase (HDAC) inhibitor in a subject comprising: a) administering a therapeutically effective amount of an HDAC inhibitor to a subject; b) taking a biological sample from the subject; c) determining whether an HDAC biomarker peptide comprising (or consisting of) an amino acid sequence selected from the group consisting of SEQ ID NOs: 1-15721 is present in the sample; and d) concluding that the HDAC treatment is efficient if an HDAC biomarker peptide comprising (or consisting of) an amino acid sequence selected from the group consisting of SEQ ID NOs: 1-15721 is present in the sample, and concluding that the HDAC treatment is not efficient if an HDAC biomarker peptide comprising (or consisting of) an amino acid sequence selected from the group consisting of SEQ ID NOs: 1-15721 is not present in the sample. Preferably, the HDAC inhibitor is selected from the group consisting of: Compound A, Compound B, and Tubastatin A. Preferably, the HDAC inhibitor is an HDAC6 inhibitor. Preferably, the HDAC6 inhibitor is selected from the group consisting of Compound B, and Tubastatin A. Preferably, the biological sample is a bone marrow sample. In one embodiment, the biological sample is a subcellular fraction (e.g., cytoplasm, soluble nuclear extract, or insoluble nuclear pellet). Preferably, the determining step utilizes an antibody.

A further embodiment of the invention comprises a method for monitoring the treatment efficiency of a histone deacetylase (HDAC) inhibitor in a subject comprising: a) administering a therapeutically effective amount of an HDAC inhibitor to a subject; b) taking a biological sample from the subject; c) determining whether an HDAC biomarker peptide comprising (or consisting of) an amino acid sequence selected from the group consisting of SEQ ID NOs: 1-15721 is present in the sample; and d) concluding that the HDAC treatment is efficient if the level of an HDAC biomarker peptide comprising (or consisting of) an amino acid sequence selected from the group consisting of SEQ ID NOs: 1-15721 in the biological sample is modulated relative to a control sample, and concluding that the HDAC treatment is not efficient if the level of an HDAC biomarker peptide comprising (or consisting of) an amino acid sequence selected from the group consisting of SEQ ID NOs: 1-15721 in the biological sample is not modulated relative to the control sample. Preferably, the HDAC inhibitor is selected from the group consisting of: Compound A, Compound B, and Tubastatin A. Preferably, the HDAC inhibitor is an HDAC6 inhibitor. Preferably, the HDAC6 inhibitor is selected from the group consisting of Compound B, and Tubastatin A. Preferably, the biological sample is a bone marrow sample. In one embodiment, the biological sample is a subcellular fraction (e.g., cytoplasm, soluble nuclear extract, or insoluble nuclear pellet). Preferably, the determining step utilizes an antibody. Preferably, the control sample is from a healthy subject. In one embodiment, the level of HDAC biomarker peptide in the sample is increased relative to the control sample. In another embodiment, the level of HDAC biomarker peptide in the biological sample is decreased relative to the control sample.

### DETAILED DESCRIPTION OF THE INVENTION

### Definitions

Unless defined otherwise, all technical and scientific terms used herein have the same meaning as is commonly understood by one of ordinary skill in the art.

The articles “a” and “an” are used herein to refer to one or more than one (i.e., to at least one) of the grammatical object of the article. By way of example, “a biomarker” means one biomarker or more than one biomarker.

The terms “administer” or “administration” refer to the act of injecting or otherwise physically delivering a substance as it exists outside the body (e.g., a formulation of the invention) into a patient, such as by mucosal, intradermal, intravenous, intramuscular delivery and/or any other method of physical delivery described herein or known in the art. When a disease, or a symptom thereof, is being treated, administration of the substance typically occurs after the onset of the disease or symptoms thereof. When a disease, or symptoms thereof, is being prevented, administration of the substance typically occurs before the onset of the disease or symptoms thereof.

Unless specifically indicated otherwise, the term “antibody,” as used herein, shall be understood to encompass antibody molecules comprising two immunoglobulin heavy chains and two immunoglobulin light chains (i.e., “full antibody molecules”) as well as antigen-binding fragments thereof. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and (optionally) constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

An antibody that “binds” an antigen of interest is one capable of binding that antigen with sufficient affinity such that the antibody is useful in detecting the presence of the antigen.

The term “biological sample” shall generally mean any biological sample obtained from an individual, body fluid, cell line, tissue culture, or other source. Body fluids are, for example, blood, lymph, sera, plasma, urine, semen, synovial fluid, and spinal fluid. Methods for obtaining tissue biopsies and body fluids from mammals are well known in the art. If the term “sample” is used alone, it shall still mean that the “sample” is a “biological sample”, i.e., the terms are used interchangeably.

The terms “composition” and “formulation” are intended to encompass a product containing the specified ingredients (e.g., an HDAC inhibitor) in, optionally, the specified amounts, as well as any product which results, directly or indirectly, from the combination of the specified ingredients in, optionally, the specified amounts.

The term “excipients” refers to inert substances that are commonly used as a diluent, vehicle, preservative, binder, stabilizing agent, etc. for drugs and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). See, also, Remington's Pharmaceutical Sciences (1990) Mack Publishing Co., Easton, Pa., which is hereby incorporated by reference in its entirety.

The phrases “respond to treatment with a HDAC inhibitor” or “respond to treatment with a HDAC6 inhibitor” or similar phrases refer to the clinical benefit imparted to a patient suffering from a disease or condition, such as cancer, from or as a result of the treatment with the HDAC inhibitor (e.g., a HDAC6 inhibitor). A clinical benefit includes a complete remission, a partial remission, a stable disease (without progression), progression-free survival, disease free survival, improvement in the time-to-progression (of the disease), improvement in the time-to-death, or improvement in the overall survival time of the patient from or as a result of the treatment with the HDAC inhibitor. There are criteria for determining a response to therapy and those criteria allow comparisons of the efficacy to alternative treatments (Slapak and Kufe, Principles of Cancer Therapy, in Harrisons's Principles of Internal Medicine, 13th edition, eds. Isselbacher et al., McGraw-Hill, Inc., 1994). For example, a complete response or complete remission of cancer is the disappearance of all detectable malignant disease. A partial response or partial remission of cancer may be, for example, an approximately 50 percent decrease in the product of the greatest perpendicular diameters of one or more lesions or where there is not an increase in the size of any lesion or the appearance of new lesions.

The term “progression of cancer” includes and may refer to metastasis, a recurrence of cancer, or an at least approximately 25 percent increase in the product of the greatest perpendicular diameter of one lesion or the appearance of new lesions. The progression of cancer is “inhibited” if recurrence or metastasis of the cancer is reduced, slowed, delayed, or prevented.

The terms “marker protein”, “marker polypeptide”, “biomarker protein”, or “biomarker polypeptide” include a protein or polypeptide that is a useful indicator of treatment efficiency in multiple myeloma.

A “kit” is any manufacture (e.g., a package or container) comprising at least one reagent, e.g., an antibody, for specifically detecting a biomarker protein or polypeptide.

As used herein, the term “small molecule” refers to a non-peptidic, non-oligomeric organic compound either synthesized in the laboratory or found in nature. Small molecules, as used herein, can refer to compounds that are “natural product-like”, however, the term “small molecule” is not limited to “natural product-like” compounds. Rather, a small molecule is typically characterized in that it contains several carbon-carbon bonds, and has a molecular weight of less than 1500, although this characterization is not intended to be limiting for the purposes of the present invention. Examples of “small molecules” that occur in nature include, but are not limited to, taxol, dynemicin, and rapamycin. In certain other preferred embodiments, natural-product-like small molecules are utilized.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. Particularly, in embodiments the compound is at least 85% pure, more preferably at least 90% pure, more preferably at least 95% pure, and most preferably at least 99% pure.

The term “therapy” refers to any protocol, method, and/or agent that can be used in the prevention, management, treatment, and/or amelioration of a disease.

The term “therapeutically effective amount” means an amount of a drug that causes a measurable effect in a subject, such as an amount effective for killing or inhibiting the growth of tumor cells.

“Treat”, “treating”, and “treatment” refer to a method of alleviating or abating a disease and/or its attendant symptoms.

The term “treatment efficiency” means how well a drug is doing its job, i.e, acting upon a target to produce a therapeutic effect.

### Stable Isotope Labeling by Amino Acids in Cell Culture (SILAC)

An embodiment of the invention comprises utilizing the SILAC method in order to quantitate changes in protein expression between two or more populations of cells. The SILAC method is a technique based on mass spectrometry that detects differences in protein abundance among samples using non-radioactive isotopic labeling. The SILAC method is known in the art, and described in detail in Ong et al., “Stable Isotope Labeling by Amino Acids in Cell Culture, SILAC, as a Simple and Accurate Approach to Expression Proteomics”, Molecular & Cellular Proteomics, vol. 1, pp. 376-386 (2002), which is incorporated herein by reference in its entirety.

Briefly, the SILAC method involves growing two populations of cells in cell culture. The first population of cells is grown in growth media containing normal amino acids (“normal media”). The second population of cells is grown in media lacking a standard essential amino acid (such as lysine), but is then supplemented with a non-radioactive, isotopically labeled form of that essential amino acid (such as lysine labeled with ¹³C atoms, rather than normal ¹²C atoms), which is called “heavy media”. When the two populations of cells are each growing in the medium, they incorporate the amino acids into all of their proteins. The growth of cells maintained in the heavy media is no different from that in normal media. All peptides containing the heavy amino acids are heavier than their normal counterparts. The cells from each of the two populations of cells are lysed. The lysates from each of the two cell populations are then combined. The combined cell lysates are then analyzed by mass spectrometry. Pairs of chemically identical peptides of different stable isotope composition can be differentiated in a mass spectrometer due to their difference in mass. The ratio of peak intensities in the mass spectrum for such peptide pairs reflects the abundance ratio for the two proteins.

The SILAC method involves a control population of cells and a test population of cells. The test population of cells may be subject to treatment with a drug or some other outside stimulus.

Preferably, the test population of cells is treated with an HDAC inhibitor. Examples of HDAC inhibitors include, but are not limited to, hydroxamic acids, cyclic tetrapeptides, benzamides, electrophilic ketones, and aliphatic acid compounds.

More preferably, the test population of cells is treated with an HDAC6 inhibitor. Examples of HDAC6 inhibitors include, but are not limited to, Compound B and Tubastatin A.

Alternatively, the test population of cells is treated with an HDAC inhibitor that inhibits HDACs 1, 2, 3, and 6. An example of such an HDAC inhibitor is Compound A.

The cells may be any type of cells, which will depend upon what is to be studied. However, both populations of cells should contain the same type of cell. Preferably, a cancer cell line is used in the SILAC method. For example, the cell line can be specific for breast cancer, hematologic cancer, colorectal cancer, lung cancer, skin cancer, brain cancer, renal cancer, liver cancer, prostate cancer, ovarian cancer, and stomach cancer. However, cell lines specific for other forms of cancer may be used.

The cells are grown in growth media. If the set of cells contains a control group of cells and a test group of cells, then one group of cells will be grown in growth media containing normal amino acids, and the other group of cells will be grown in media lacking a standard essential amino acid that is then supplemented with a non-radioactive, isotopically labeled form of that essential amino acid (such as an amino acid labeled with ¹³C atoms, rather than normal ¹²C atoms).

The heavy media lacks an essential amino acid, but is then supplemented with a non-radioactive, isotopically labeled form of that essential amino acid. Of the 20 standard amino acids, nine are essential amino acids. The nine essential amino acids are: histidine, isoleucine, leucine, lysine, methionine, phenylalanine, threonine, tryptophan, and valine. The heavy media may lack any one or more of the above essential amino acids. Preferably, the heavy media lacks normal lysine. However, the heavy media must then be supplemented with a non-radioactive, isotopically labeled form of that essential amino acid. Preferably, the non-radioactive, isotopically labeled essential amino acid is ¹³C labeled lysine.

The isotope label may include specific heavy isotopes of elements present in biomolecules such as ²H, ³H, ¹³C, ¹⁴C, ¹⁵N, ¹⁸O, ³²P, ³³P, ³³S, ³⁴S, ¹²⁵I, or ¹³¹I. Thus the essential amino acid may be labeled with any one of the above isotopes, or another isotope.

The cells may be lysed by any means known in the art.

The performance of mass spectrometry is known in the art.

### Cytoplasm (C), Soluble Nuclear Extract (N), and Insoluble Nuclear Pellet (P) Analysis (CNP Analysis)

An embodiment of the invention comprises fractionating a population of cells, such as a test group of cells, into three subcellular fractions—cytoplasm (C), soluble nuclear extract (N), and insoluble nuclear pellet (P).

The SILAC method is performed as described above.

The harvested “heavy” and “light” labeled cells are lysed. The supernatants from each are then saved after centrifugation. The protein concentration in both the “heavy” or “light” labeled supernatant is measured. Equal amounts of crude proteins in the supernatants are mixed, and the crude proteins are precipitated (this is called the cytosolic fraction). After washing, the protein pellets are dissolved for trypsin digestion.

The remaining cell pellets are further lysed. The resulting lysates are clarified by centrifugation, and the supernatant is saved as nuclear extracts fractions. The protein concentration in “heavy” or “light” labeled supernatant is measured. Equal amounts of crude proteins in supernatant are mixed, and the crude proteins are precipitated (this is called the nuclear extracts fraction). After washing, the proteins pellets are dissolved for trypsin digestion.

The final remaining cell pellets are dissolved in urea to extract the chromatin-binding proteins. The protein concentration is then measured. Then, equal amounts of chromatin-binding proteins in urea solution are mixed, and the proteins are precipitated (this is called the nuclear pellet fraction). After washing, the proteins pellets are dissolved for trypsin digestion.

After trypsin digestion for the above three fractions, the Kac affinity enrichment followed by MS analysis and data inquiry are separately performed. The combined Kac data formed the total Kac profiling data in the pair of Compound B vs DMSO.

Any peptides identified as drug specific, such as HDAC specific, in the SILAC method described above with this subcellular localization will allow one to understand the peptide's function inside the cell.

### Biological Pathway Analysis

Once biomarker peptides are identified for a specific drug by the SILAC method, the biomarker peptides may be compared to peptides/proteins in a biological pathway database in order to identify major biological pathways or functions implicated by the drug's action. A preferred pathway analysis website is at www dot broadinstitute dot org/gsea/index dot jsp. Such analysis will be related to the drug's (e.g., HDAC or HDAC6) specific clinical effects.

### Analysis of Total Peptide (Including Both Acetylated and Unacetylated) Amount in Compound B Treated Cell Lysate

The SILAC method is performed as described above. The Compound B- (¹²C-lysine labeled) and DMSO-treated (¹³C-lysine labeled) cells are lysed. The samples are sonicated, and centrifuged to remove remaining debris. Protein content in the supernatant is determined Crude protein from each sample is mixed and separated. After electrophoresis, the gel is stained. The entire gel lane is cut into slices and digested with trypsin. The gel bands are cut into small cubes and washed. The gel pieces are dehydrated. Supernatant is discarded and the gel pieces are vacuum-dried. Disulfide bonds are cleaved and then alkylated. The gel pieces are dehydrated and vacuum-dried again. Gel pieces are covered with trypsin solution (10 ng/μl in 50 mM ammonium bicarbonate buffer). After incubation, the remaining trypsin solution is removed. The peptides in the gels are extracted. All the supernatant is combined and vacuum-dried followed by mass spectrometer analysis.

### Histone Deacetylase (HDAC) Inhibitors

An HDAC inhibitor useful in the SILAC method and in the Examples herein can be any HDAC inhibitor, such as a small molecule organic compound, an antibody, a siRNA, an aptamer, a nucleic acid, a protein, or a peptide. Preferably, the HDAC inhibitor is a small molecule organic compound.

Preferably, the HDAC inhibitor is an HDAC6 inhibitor. This means that the HDAC inhibitor selectively inhibits HDAC6 over other forms of HDAC.

In a specific embodiment, an HDAC inhibitor has the following chemical structure:

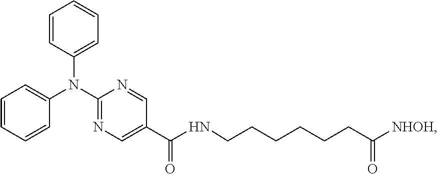

which is referred to herein as Compound A. Methods for making Compound A are known in the art.

In another specific embodiment, an HDAC 6 inhibitor has the following chemical structure:

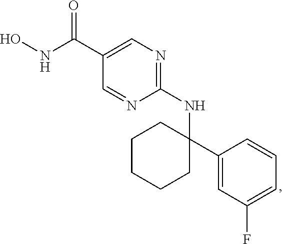 [SMILES: O=C(NO)c1cnc(NC2(c3cccc(F)c3)CCCCC2)nc1]

which is referred to herein as Compound B. Methods for making Compound B are known in the art.

In yet another specific embodiment, an HDAC 6 inhibitor has the following chemical structure:

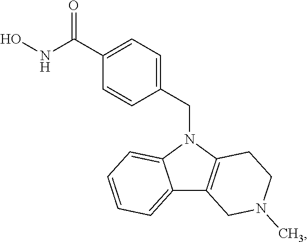 [SMILES: CN1CCc2c(c3ccccc3n2Cc2ccc(C(=O)NO)cc2)C1]

which is referred to herein as Tubastatin A. Methods for making Tubastatin A are known in the art.

HDAC inhibitors have one or more of the following properties: the compound is capable of inhibiting at least one histone deacetylase; the compound is capable of inhibiting HDAC6; the compound is a selective HDAC6 inhibitor; the compound binds to the poly-ubiquitin binding domain of HDAC6; the compound is capable of inducing apoptosis in cancer cells (especially multiple myeloma cells, non-Hodgkin's lymphoma (NML) cells, breast cancer cells, acute myelogeous leukemia (AML) cells); and/or the compound is capable of inhibiting aggresome formation.

An HDAC inhibitor may comprise a metal binding moiety, preferably a zinc-binding moiety such as a hydroxamate. Certain hydroxamates are potent inhibitors of HDAC6 activity; without wishing to be bound by theory, it is believed that the potency of these hydroxamates is due, at least in part, to the ability of the compounds to bind zinc. An HDAC inhibitor may include at least one portion or region that can confer selectivity for a biological target implicated in the aggresome pathway, e.g., a biological target having tubulin deacetylase (TDAC) or HDAC activity, e.g., HDAC6. Thus, some HDAC inhibitors include a zinc-binding moiety spaced from other portions of the molecule that are responsible for binding to the biological target.

### Biomarkers

As shown in the Examples herein, the SILAC method may be used to identify biomarkers that tell us why a particular set of cells is killed. That is, the biomarkers are indicative of HDAC inhibitors and cell death in myeloma.

The set of cells may contain a control group of cells and a test group of cells. This set of cells allows one to determine how a particular drug works in a particular type of cancer cell.

Preferably, the biomarkers are selected from the peptides in Table 1 (which are identical to the peptides in Table 2) and Table 9.

### Kits

Certain embodiments of the invention include kits that may be used in the experimental methods in order to identify drug specific and disease specific biomarkers.

For example, in a certain embodiment of the invention, the invention provides a kit comprising one or more of the following: a drug; a cell line; cell growth media containing normal amino acids; cell growth media lacking a standard essential amino acid; a non-radioactive, isotopically labeled form of that essential amino acid; a cell lysis buffer; a protease such as trypsin; a non-specific anti-acetylated amino acid antibody cocktail; a detection agent; and instructions for performing the SILAC method and identifying a biomarker peptide of the invention.

### Methods

An embodiment of the invention provides a method for monitoring the treatment efficiency of a drug in a subject. Preferably, the drug is an HDAC inhibitor.

In a specific embodiment, the invention provides a method for monitoring the treatment efficiency of a histone deacetylase (HDAC) inhibitor in a subject comprising:

a) administering a therapeutically effective amount of an HDAC inhibitor to a subject;
b) taking a biological sample from the subject;
c) determining whether an HDAC biomarker peptide comprising an amino acid sequence selected from the group consisting of SEQ ID NOs: 1-15721 is present in the sample; and
d) concluding that the HDAC treatment is efficient if an HDAC biomarker peptide comprising an amino acid sequence selected from the group consisting of SEQ ID NOs: 1-15721 is present in the sample, and concluding that the HDAC treatment is not efficient if an HDAC biomarker peptide comprising an amino acid sequence selected from the group consisting of SEQ ID NOs: 1-15721 is not present in the sample.

The method will monitor whether the HDAC treatment that a patient receives is efficient. That is, the method will be indicative of HDAC inhibition and cell death.

As discussed above, the HDAC inhibitor may be any HDAC inhibitor known in the art. Preferably, the HDAC inhibitor is selected from the group consisting of: Compound A, Compound B, and Tubastatin A. Preferably, the HDAC inhibitor is an HDAC6 inhibitor. More preferably, the HDAC6 inhibitor is selected from the group consisting of Compound B, and Tubastatin A.

The HDAC inhibitor may be administered using any therapeutically effective amount and any route of administration.

The method involves taking a biological sample from a patient in order to determine the treatment efficiency of a drug in a patient with a particular disease. For example, the biological sample may be a sample from whole blood, blood serum, blood plasma, semen, urine, mucus, bone marrow, or other body sample. In a preferred embodiment, the biological sample is from bone marrow.

The determining step may use any means or detection agent known in the art to identify an acetylated or unacetylated peptide of the invention. Preferably, the determining step utilizes an antibody. More preferably, the determining step utilizes a non-specific anti-acetylated lysine antibody cocktail to identify acetylated biomarker peptides of the invention. Alternatively, peptide specific antibodies may be used to identify the unacetylated biomarker peptides of the invention.

All patents, patent applications, and publications mentioned herein are each hereby incorporated by reference in their entirety.

### EXAMPLES

The following examples are provided to aid in the understanding of the invention. It is understood that modifications can be made to the procedures set forth below without departing from the spirit and scope of the invention.

Conventional techniques of molecular biology and nucleic acid/protein chemistry, which are within the skill of the art, are explained in the literature and used in the practice of the invention. See, for example, Sambrook, J. et al., Molecular Cloning: A Laboratory Manual, Cold Spring Harbor Laboratory Press, Cold Spring Harbor, N.Y., 1989; Gait, M. J. (ed.), Oligonucleotide synthesis—a practical approach, IRL Press Limited, 1984; Hames, B. D. and Higgins, S. J. (eds.), Nucleic acid hybridisation—a practical approach, IRL Press Limited, 1985; and a series, Methods in Enzymology, Academic Press, Inc., all of which are incorporated herein by reference.

### Example 1

### SILAC Method

This example describes SILAC studies that were performed to compare the effects of three different HDAC inhibitors (Compound A, Compound B, and tubastatin A) to a control on a human multiple myeloma cell line. The HDAC inhibitors were also compared amongst themselves to determine differences in their effects.

A human myeloma cell line MM.1S was grown using either ¹²C (light) or ¹³C (heavy) lysine containing culture medium. The “heavy” cells were treated with DMSO as the control, and the “light” cells were treated with a single HDAC inhibitor at 37° C. for 6 hours. The heavy and light cells were lysed, and the lysates were mixed in 1:1 ratio and digested with the protease trypsin. The acetylated lysine containing peptides (due to the action of the HDAC inhibitor) in the digested peptide pool were captured by a non-specific anti-acetylated lysine antibody cocktail (the antibodies identify both light and heavy acetylated peptides), and subjected to mass spectrometry analysis in order to identify thousands of individual peptides.

Thousands of peptides were identified in pairs (¹²C and ¹³C) due to their known mass differences. The amino acid sequence and quantitative ratio (Light/Heavy) of a given peptide in two differentially treated cell lysates were then determined and annotated with their possible protein (gene) sources.

As stated above, three HDAC inhibitors (Compound A, Compound B, and Tubastatin A) were used. Thus, the SILAC method was performed three times, with a different HDAC inhibitor compared to a control each time. Accordingly, there are three pairs of quantitative data in the table below, i.e., Compound A:DMSO, Compound B:DMSO, and Tubastatin A:DMSO. A cut-off of 1.30 fold of change (increase or decrease) was then used to determine “changed” or “+” in the table, and any peptides with a fold of change less than 1.3 fold were determined to be “unchanged” or “0” in the table. Those peptides that were not identified were marked as “NA” in the table.

Since Compound A is able to inhibit HDAC1, 2, 3 and 6, and Compound B and Tubastatin A can only inhibit HDAC6 at the concentration used in this study, any AcKs (acetylated lysine) in Compound B or Tubastatin A can be referred to as HDAC6 specific. Although both Compound B and Tubastatin A are selective HDAC6 inhibitors, some previous cell biology studies have shown them to have differential activities. As a result, both compounds were used in order to investigate their similarities and differences.

The results of these studies are shown in Table 1 below.

The first column of Table 1 shows the amino acid sequence of the acetylated peptide that was identified by the SILAC method, wherein “(ac)” means that the previous amino acid was acetylated.

The second, third, and fourth columns of Table 1 show data for Compound A, Tubastatin A, and Compound B, respectively. The numbers in these columns represent the light to heavy ratio (L/H ratio) for each peptide for each drug treated group of cells. The numbers are in a log 2 scale so 1 represents a 2× change, 0 represents no change, and NA represents unknown.

The fifth column of Table 1 shows the gene name and position information, which is in parentheses, for the acetylated lysine. One number in parentheses means that only one lysine was acetylated. Two numbers in parentheses means that two lysines were acetylated.

The sixth column of Table 1 shows the gene symbol for the peptide in the fifth column.

| | Cmpd | Tub | Cmpd | | |
|---|---|---|---|---|---|
| Peptide | A | A | B | Protein_Position | Gene |
| FK(ac)ESFAEMNR (SEQ ID NO: 1) | 0.00 | 0.52 | −0.67 | Cytochrome c oxidase subunit 4 | COX4I1 |
| | | | | isoform 1 mitochondrial [87] | |
| ELNK(ac)ILEGR (SEQ ID NO: 2) | 0.00 | 0.45 | 0.47 | Dihydrolipoyllysine-residue | DLAT |
| | | | | acetyltransferase component of | |
| | | | | pyruvate dehydrogenase complex | |
| | | | | mitochondrial [466] | |
| AAEVLNK(ac)HSLSGRPLK | 0.00 | −0.87 | −0.74 | Isoform 1 of Heterogeneous nu- | HNRNPM |
| (SEQ ID NO: 3) | | | | clear ribonucleoprotein M [134] | |
| LYK(ac)EELEQTYHAK | 0.00 | 0.46 | 0.48 | Lamin-B1 [261] | LMNB1 |
| (SEQ ID NO: 4) | | | | | |
| TLVLSDK(ac)HSPQK(ac)K | 0.00 | 0.67 | 0.38 | Isoform 1 of Histone-lysine N- | MLL3 |
| (SEQ ID NO: 5) | | | | methyltransferase MLL3 | |
| | | | | [2809:2814] | |
| (ac)CNTPTYCDLGK(ac)AAK | 0.00 | 0.52 | −0.63 | Isoform 1 of Voltage-dependent | VDAC3 |
| (SEQ ID NO: 6) | | | | anion-selective channel protein | |
| | | | | 3 [12] | |
| LGTDESK(ac)FNAVLCSR | NA | −0.44 | 0.50 | cDNA FLJ55482 highly similar | ANXA11 |
| (SEQ ID NO: 7) | | | | to Annexin A11 [478] | |
| SFDSEFK(ac)LACK | NA | 0.44 | 0.51 | Cytoplasmic dynein 1 heavy | DYNC1H1 |
| (SEQ ID NO: 8) | | | | chain 1 [4283] | |
| EPVAVLK(ac)ANR | NA | −0.58 | −0.40 | Beta-hexosaminidase subunit | HEXB |
| (SEQ ID NO: 9) | | | | beta [161] | |
| LNK(ac)DQWEER | NA | −0.38 | 0.71 | Moesin [165] | MSN |
| (SEQ ID NO: 10) | | | | | |
| DFLAGGVAAAISK(ac)TAVAPIER | NA | 0.80 | −0.47 | ADP/ATP translocase 2 [23] | SLC25A5 |
| (SEQ ID NO: 11) | | | | | |
| YYNK(ac)YINVK | 0.54 | 0.66 | −0.43 | Isoform 1 of ATP synthase | ATP5J2 |
| (SEQ ID NO: 12) | | | | subunit f mitochondrial [54] | |
| TNK(ac)NK(ac)SSISR | 1.00 | 0.64 | 0.62 | CREB-binding protein | CREBBP |
| (SEQ ID NO: 13) | | | | [1595:1597] | |
| TSK(ac)NK(ac)SSLSR | 1.00 | 0.42 | 0.43 | Histone acetyltransferase | EP300 |
| (SEQ ID NO: 14) | | | | p300 [1558:1560] | |
| AGGK(ac)AGK(ac)DSGK | 1.00 | 0.47 | 0.95 | Histone H2A.V [5:8] | H2AFV |
| (SEQ ID NO: 15) | | | | | |
| PDPAK(ac)SAPAPK(ac)K | 1.00 | 0.47 | 0.66 | Histone H2B type 1-O [6:12] | HIST1H2BO |
| (SEQ ID NO: 16) | | | | | |
| YLTNQK(ac)NSNSK(ac)NDR | 1.00 | 0.58 | 0.43 | Isoform 3 of Chromatin | MEAF6 |
| (SEQ ID NO: 17) | | | | modification-related | |
| | | | | protein MEAF6 [69:74] | |
| GVTQFGNK(ac)YIQQTKPLTLER | 1.00 | 0.50 | 0.50 | Cleavage and polyadenylation | NUDT21 |
| (SEQ ID NO: 18) | | | | specificity factor subunit | |
| | | | | 5 [23] | |
| IHNFGLIQEK(ac)LAR | 0.00 | −0.60 | NA | cDNA FLJ56425 highly similar to | ACADVL |
| (SEQ ID NO: 19) | | | | Very-long-chain specific acyl- | |
| | | | | CoAdehydrogenase mitochondrial | |
| | | | | [428] | |
| LDHLAEK(ac)FR (SEQ ID NO: 20) | 0.00 | −0.51 | 0.00 | Isoform 1 of Alpha-actinin-1 | ACTN1 |
| | | | | [398] | |
| LK(ac)DISTLEPLKK | 0.00 | −0.56 | 0.00 | Isoform 1 of Acidic leucine-rich | ANP32B |
| (SEQ ID NO: 21) | | | | nuclear phosphoprotein 32 family | |
| | | | | member B [101] | |
| QRQEVCQSYK(ac)SLYGK | 0.00 | 0.68 | 0.00 | Annexin A6 [63] | ANXA6 |
| (SEQ ID NO: 22) | | | | | |
| MYFDK(ac)YVLKPATEGK | 0.00 | 0.62 | 0.00 | Isoform 2 of ATPase family AAA | ATAD3A |
| (SEQ ID NO: 23) | | | | domain-containing protein | |
| | | | | 3A [491] | |
| IVEYEK(ac)EMEK | 0.00 | 0.63 | 0.00 | Isoform 1 of ATP synthase | ATP5H |
| (SEQ ID NO: 24) | | | | subunit d mitochondrial [117] | |
| FYGDEEK(ac)DKGLQTSQDAR | 0.00 | −0.97 | 0.00 | Calreticulin [62] | CALR |
| (SEQ ID NO: 25) | | | | | |
| ALEHFTDLYDIK(ac)R | 0.00 | −0.62 | 0.00 | Isoform 1 of Clathrin heavy | CLTC |
| (SEQ ID NO: 26) | | | | chain 1 [637] | |
| KYEFSPQTLCCYGK(ac)QLCTIPR | 0.00 | 0.60 | 0.00 | CREB-binding protein [1216] | CREBBP |
| (SEQ ID NO: 27) | | | | | |
| ALK(ac)DLYCHK | 0.00 | 0.43 | 0.00 | cAMP-responsive element modu- | CREM |
| (SEQ ID NO: 28) | | | | lator isoform s [240] | |
| LYCPK(ac)CMDVYTPK | 0.00 | −0.39 | 0.00 | Casein kinase II subunit | CSNK2B |
| (SEQ ID NO: 29) | | | | beta [139] | |
| FK(ac)DPNCVGTVLASR | 0.00 | −0.51 | 0.00 | Isoform 1 of DAZ-associated | DAZAP1 |
| (SEQ ID NO: 30) | | | | protein 1 [59] | |
| APTIVGK(ac)SSLNPILFR | 0.00 | −0.51 | 0.00 | Dolichyl-diphosphooligo- | DDOST |
| (SEQ ID NO: 31) | | | | saccharide--protein glycosyl | |
| | | | | transferase 48 kDa subunit [189] | |
| FEK(ac)NFYVEHPEVAR | 0.00 | −0.42 | 0.00 | probable ATP-dependent RNA | DDX17 |
| (SEQ ID NO: 32) | | | | helicase DDX17 isoform 3 [132] | |
| DWVLNEFK(ac)HGK | 0.00 | −0.41 | 0.00 | Probable ATP-dependent RNA | DDX5 |
| (SEQ ID NO: 33) | | | | helicase DDX5 [388] | |
| FGNPGEK(ac)LVK (SEQ ID NO: 34) | 0.00 | 0.45 | NA | Probable ATP-dependent RNA | DDX5 |
| | | | | helicase DDX5 [40] | |
| VVK(ac)HPIFER (SEQ ID NO: 35) | 0.00 | −0.91 | 0.00 | DnaJ homolog subfamily B member | DNAJB11 |
| | | | | 11 [247] | |
| TCEESSFCK(ac)R (SEQ ID NO: 36) | 0.00 | −0.51 | NA | Isoform 2 of Neutral alpha- | GANAB |
| | | | | glucosidase AB [48] | |
| ASGLAAGK(ac)GVIVAK | 0.00 | 0.46 | 0.00 | Isoform Long of Trifunctional | GART |
| (SEQ ID NO: 37) | | | | purine biosynthetic protein | |
| | | | | adenosine-3 [156] | |
| IIVDELK(ac)QEVISTSSK | 0.00 | −0.62 | 0.00 | Guanine nucleotide-binding | GNB2L1 |
| (SEQ ID NO: 38) | | | | protein subunit beta- | |
| | | | | 2-like 1 [271] | |
| LVK(ac)VWNLANCK | 0.00 | −0.70 | 0.00 | Guanine nucleotide-binding | GNB2L1 |
| (SEQ ID NO: 39) | | | | protein subunit beta- | |
| | | | | 2-like 1 [175] | |
| (ac)AHYNFK(ac)K | 0.00 | −0.38 | 0.00 | Nucleolar GTP-binding | GTPBP4 |
| (SEQ ID NO: 40) | | | | protein 1 [7] | |
| GQQQVFK(ac)GLNDK | 0.00 | 0.46 | 0.00 | Trifunctional enzyme subunit | HADHA |
| (SEQ ID NO: 41) | | | | alpha mitochondrial [406] | |
| K(ac)HPDSSVNFAEFSK | 0.00 | −0.48 | 0.00 | High mobility group protein | HMGB2 |
| (SEQ ID NO: 42) | | | | B2 [30] | |
| RGEEGHDPK(ac)EPEQLR | 0.00 | −0.45 | 0.00 | Isoform 1 of Heterogeneous nuclear | HNRNPA3 |
| (SEQ ID NO: 43) | | | | ribonucleoprotein A3 [29] | |
| AFITNIPFDVK(ac)WQSLK | 0.00 | −0.48 | 0.00 | Isoform 1 of Heterogeneous nuclear | HNRNPM |
| (SEQ ID NO: 44) | | | | ribonucleoprotein M [83] | |
| VK(ac)VLFVR (SEQ ID NO: 45) | 0.00 | −0.77 | 0.00 | Isoform 1 of Heterogeneous nuclear | HNRNPR |
| | | | | ribonucleoprotein R [341] | |
| GYFEYIEENK(ac)YSR | 0.00 | −0.60 | 0.00 | Isoform Short of Heterogeneous | HNRNPU |
| (SEQ ID NO: 46) | | | | nuclear ribonucleoprotein U [246] | |
| TTWVTK(ac)HAAENPGK | 0.00 | −0.56 | 0.00 | Isoform Short of Heterogeneous | HNRNPU |
| (SEQ ID NO: 47) | | | | nuclear ribonucleoprotein U [497] | |
| EELVK(ac)NLGTIAK | 0.00 | −0.60 | NA | Endoplasmin [161] | HSP90B1 |
| (SEQ ID NO: 48) | | | | | |
| ELISNASDALDK(ac)IR | 0.00 | −0.53 | 0.00 | Endoplasmin [114] | HSP90B1 |
| (SEQ ID NO: 49) | | | | | |
| ETLQQHK(ac)LLK (SEQ ID NO: 50) | 0.00 | −0.74 | 0.00 | Endoplasmin [455] | HSP90B1 |
| IADDK(ac)YNDTFWK | 0.00 | −0.78 | 0.00 | Endoplasmin [479] | HSP90B1 |
| (SEQ ID NO: 51) | | | | | |
| NK(ac)EIFLR (SEQ ID NO: 52) | 0.00 | −1.00 | 0.00 | Endoplasmin [97] | HSP90B1 |
| IINEPTAAAIAYGLDK(ac)R | 0.00 | −0.54 | NA | 78 kDa glucose-regulated protein | HSPA5 |
| (SEQ ID NO: 53) | | | | [213] | |
| NQLTSNPENTVFDAK(ac)R | 0.00 | −0.74 | 0.00 | 78 kDa glucose-regulated protein | HSPA5 |
| (SEQ ID NO: 54) | | | | [96] | |
| NQVAMNPTNTVFDAK(ac)R | 0.00 | −0.48 | 0.00 | Isoform 1 of Heat shock cognate | HSPA8 |
| (SEQ ID NO: 55) | | | | 71 kDa protein [71] | |
| YAASSYLSLTPEQWK(ac)SHR | 0.00 | −0.63 | 0.00 | IGL @ protein [208] | IGLV2-14, |
| (SEQ ID NO: 56) | | | | | IGLC2 |
| IHLEIK(ac)QLNR (SEQ ID NO: 57) | 0.00 | −0.56 | NA | Protein ERGIC-53 [346] | LMAN1 |
| LYKEELEQTYHAK(ac)LENAR | 0.00 | −0.73 | 0.00 | Lamin-B1 [271] | LMNB1 |
| (SEQ ID NO: 58) | | | | | |
| GFGYK(ac)GSCFHR | 0.00 | −0.45 | 0.00 | peptidyl-prolyl cis-trans | LOC390956 |
| (SEQ ID NO: 59) | | | | isomerase A-like [71] | |
| ICCQFDFK(ac)R (SEQ ID NO: 60) | 0.00 | −0.69 | 0.00 | Alpha-mannosidase 2 [373] | MAN2A1 |
| ADKDYHFK(ac)VDNDENEHQLSLR | 0.00 | −0.64 | 0.00 | Isoform 2 of Nucleophosmin [32] | NPM1 |
| (SEQ ID NO: 61) | | | | | |
| FINYVK(ac)NCFR (SEQ ID NO: 62) | 0.00 | −0.52 | NA | Isoform 2 of Nucleophosmin [244] | NPM1 |
| SGVGNVFIK(ac)NLDK | 0.00 | −0.54 | NA | Isoform 1 of Polyadenylate- | PABPC4 |
| (SEQ ID NO: 63) | | | | binding protein 4 [104] | |
| GEK(ac)FVMQEEFSR | 0.00 | −0.58 | 0.00 | Protein disulfide-isomerase | PDIA3 |
| (SEQ ID NO: 64) | | | | A3 [335] | |
| PSHLTNK(ac)FEDK | 0.00 | −0.63 | 0.00 | Protein disulfide-isomerase | PDIA3 |
| (SEQ ID NO: 65) | | | | A3 [214] | |
| YGVSGYPTLK(ac)IFR | 0.00 | −0.60 | NA | Protein disulfide-isomerase | PDIA3 |
| (SEQ ID NO: 66) | | | | A3 [104] | |
| SLLGK(ac)DVLFLK | 0.00 | 0.38 | 0.00 | Phosphoglycerate kinase 1 [91] | PGK1 |
| (SEQ ID NO: 67) | | | | | |
| SIYGEK(ac)FEDENFILK | 0.00 | −0.67 | 0.00 | Peptidyl-prolyl cis-trans | PPIA |
| (SEQ ID NO: 68) | | | | isomerase A [82] | |
| TVPK(ac)TVDNFVALATGEK | 0.00 | −0.49 | 0.00 | Peptidyl-prolyl cis-trans | PPIB |
| (SEQ ID NO: 69) | | | | isomerase B [71] | |
| VIFGLFGK(ac)TVPK | 0.00 | −0.44 | 0.00 | Peptidyl-prolyl cis-trans | PPIB |
| (SEQ ID NO: 70) | | | | isomerase B [67] | |
| VIK(ac)DFMIQGGDFTR | 0.00 | −0.40 | 0.00 | Peptidyl-prolyl cis-trans | PPIB |
| (SEQ ID NO: 71) | | | | isomerase B [98] | |
| IYGFYDECK(ac)R (SEQ ID NO: 72) | 0.00 | −0.43 | 0.00 | Isoform Gamma-1 of | PPP1CC |
| | | | | Serine/threonine-protein | |
| | | | | phosphatase PP1-gamma catalytic | |
| | | | | subunit [141] | |
| EIDSVK(ac)YLECSALTQR | 0.00 | −0.48 | 0.00 | Ras-related C3 botulinum toxin | RAC2 |
| (SEQ ID NO: 73) | | | | substrate 2 [153] | |
| (ac)ADK(ac)EAAFDDAVEER | 0.00 | 0.71 | NA | Isoform 1 of Histone-binding | RBBP4 |
| (SEQ ID NO: 74) | | | | protein RBBP4 [4] | |
| ACFYNLDK(ac)FR (SEQ ID NO: 75) | 0.00 | −0.42 | NA | Splicing factor 45 [391] | RBM17 |
| LGLGEGAEEK(ac)SIPTLISR | 0.00 | −0.45 | NA | Isoform 2 of Regulator of | RCC1 |
| (SEQ ID NO: 76) | | | | chromosome condensation [366] | |
| TGCIGAK(ac)HR (SEQ ID NO: 77) | 0.00 | −0.47 | NA | Isoform 1 of 60S ribosomal | RPL11 |
| | | | | protein L11 [154] | |
| WFQQK(ac)YDGIILPGK | 0.00 | −0.64 | NA | Isoform 1 of 60S ribosomal | RPL11 |
| (SEQ ID NO: 78) | | | | protein L11 [169] | |
| IFAPNHVVAK(ac)SR | 0.00 | −0.51 | 0.00 | 60S ribosomal protein | RPL18A |
| (SEQ ID NO: 79) | | | | L18a [41] | |
| IEHIK(ac)HSK (SEQ ID NO: 80) | 0.00 | −0.44 | NA | 60S ribosomal protein | RPL21, |
| | | | | L21 [97] | SNORD102, |
| | | | | | SNORA27, |
| | | | | | RPL21P19 |
| YLK(ac)YLTK (SEQ ID NO: 81) | 0.00 | −0.38 | 0.00 | 60S ribosomal protein L22 [84] | RPL22 |
| INFDK(ac)YHPGYFGK | 0.00 | −0.52 | 0.00 | 60S ribosomal protein L27a [47] | RPL27A |
| (SEQ ID NO: 82) | | | | | |
| FIDTTSK(ac)FGHGR | 0.00 | −0.45 | 0.00 | 60S ribosomal protein | RPL3L |
| (SEQ ID NO: 83) | | | | L3-like [373] | |
| AASLK(ac)SNYNLPMHK | 0.00 | −0.57 | 0.00 | 60S ribosomal protein L4 [274] | RPL4 |
| (SEQ ID NO: 84) | | | | | |
| LNILK(ac)LAPGGHVGR | 0.00 | −0.48 | NA | 60S ribosomal protein L4 [239] | RPL4 |
| (SEQ ID NO: 85) | | | | | |
| NFLGEK(ac)YIR (SEQ ID NO: 86) | 0.00 | −0.44 | 0.00 | 60S ribosomal protein L9 [121] | RPL9 |
| VANVSLLALYK(ac)GK | 0.00 | −0.46 | 0.00 | 40S ribosomal protein S23 [135] | RPS23 |
| (SEQ ID NO: 87) | | | | | |
| LAVLK(ac)YYK (SEQ ID NO: 88) | 0.00 | −0.52 | 0.00 | Ubiquitin-40S ribosomal protein | RPS27A |
| | | | | S27a [104] | |
| LK(ac)YALTGDEVKK | 0.00 | −0.38 | 0.00 | 40S ribosomal protein S4 X | RPS4X |
| (SEQ ID NO: 89) | | | | isoform [53] | |
| SFQK(ac)IQVR (SEQ ID NO: 90) | 0.00 | −0.51 | 0.00 | 40S ribosomal protein S7 [74] | RPS7 |
| ELLTLDEK(ac)DPR | 0.00 | −0.52 | NA | 40S ribosomal protein S9 [66] | RPS9 |
| (SEQ ID NO: 91) | | | | | |
| LQTQVFK(ac)LGLAK | 0.00 | −0.51 | NA | 40S ribosomal protein S9 [116] | RPS9 |
| (SEQ ID NO: 92) | | | | | |
| (ac)ATETVELHK(ac)LK | 0.00 | −0.85 | 0.00 | SAP domain-containing | SARNP |
| (SEQ ID NO: 93) | | | | ribonucleoprotein [10] | |
| LAELK(ac)QECLAR | 0.00 | −0.73 | 0.00 | SAP domain-containing | SARNP |
| (SEQ ID NO: 94) | | | | ribonucleoprotein 17] | |
| ANLVFK(ac)EIEK (SEQ ID NO: 95) | 0.00 | −0.49 | NA | Isoform SCPx of Non-speific | SCP2 |
| | | | | lipid-transfer protein [438] | |
| EGQNWLEK(ac)K (SEQ ID NO: 96) | 0.00 | 0.48 | NA | Signal peptidase complex catalytic | SEC11C |
| | | | | subunit SEC11C [147] | |
| GFGFIK(ac)LESR (SEQ ID NO: 97) | 0.00 | −0.60 | NA | Isoform Long of Splicing factor | SFPQ |
| | | | | proline- and glutamine-rich [338] | |
| LFAK(ac)YGEPGEVFINK | 0.00 | −0.38 | 0.00 | Isoform Long of Splicing factor | SFPQ |
| (SEQ ID NO: 98) | | | | proline- and glutamine-rich [319] | |
| YGEPGEVFINK(ac)GK | 0.00 | −0.43 | NA | Isoform Long of Splicing factor | SFPQ |
| (SEQ ID NO: 99) | | | | proline- and glutamine-rich [330] | |
| YIYDSAFHPDTGEK(ac)MILIGR | 0.00 | 0.55 | 0.00 | Sideroflexin-1 [86] | SFXN1 |
| (SEQ ID NO: 100) | | | | | |
| LVESLPQEIK(ac)ANVAK | 0.00 | −0.56 | 0.00 | cDNA FLJ60124 highly similar to | SLC25A10 |
| (SEQ ID NO: 101) | | | | Mitochondrial dicarboxylate | |
| | | | | carrier [173] | |
| AFFK(ac)GAWSNVLR | 0.00 | 0.45 | 0.00 | ADP/ATP translocase 2 [272] | SLC25A5 |
| (SEQ ID NO: 102) | | | | | |
| YFPTQALNFAFK(ac)DK | 0.00 | 0.58 | 0.00 | ADP/ATP translocase 2 [92] | SLC25A5 |
| (SEQ ID NO: 103) | | | | | |
| TWEK(ac)LLLAAR | 0.00 | −0.45 | 0.00 | 33 kDa protein [57] | SNORA6, |
| (SEQ ID NO: 104) | | | | | RPSAP15, |
| | | | | | SNORA62, |
| | | | | | RPSA |
| YLMEEDEDAYK(ac)K | 0.00 | −0.42 | NA | 60S ribosomal protein L5 [220] | SNORD21, |
| (SEQ ID NO: 105) | | | | | RPL5 |
| TVFAEHISDECK(ac)R | 0.00 | −0.46 | NA | 60S ribosomal protein L3 [115] | SNORD43, |
| (SEQ ID NO: 106) | | | | | RPL3 |
| TKDIEDVFYK(ac)YGAIR | 0.00 | −0.64 | 0.00 | Isoform ASF-1 of Serine/arginine- | SRSF1 |
| (SEQ ID NO: 107) | | | | rich splicing factor 1 [38] | |
| (ac)AEPSAPESK(ac)HK | 0.00 | −0.42 | 0.00 | Dolichyl-diphosphooligosaccharide-- | STT3B |
| (SEQ ID NO: 108) | | | | protein glycosyltransferase | |
| | | | | subunit STT3B [10] | |
| INVYYNEATGGK(ac)YVPR | 0.00 | −0.43 | 0.00 | Tubulin beta-2C chain [58] | TUBB2C |
| (SEQ ID NO: 109) | | | | | |
| ILAEGGGAK(ac)FK | 0.00 | −0.61 | 0.00 | elongation factor Tu mitochondrial | TUFM |
| (SEQ ID NO: 110) | | | | precursor [91] | |
| QIESK(ac)TAFQEALDAAGDK | 0.00 | −0.70 | 0.00 | Thioredoxin [8] | TXN |
| (SEQ ID NO: 111) | | | | | |
| LFKPGQEAVK(ac)YQGPR | 0.00 | −0.47 | 0.00 | Thioredoxin domain-containing | TXNDC5, |
| (SEQ ID NO: 112) | | | | protein 5 [150] | MUTED, |
| | | | | | MUTED- |
| | | | | | TXNDC5 |
| VYVAK(ac)VDCTAHSDVCSAQGVR | 0.00 | −0.49 | 0.00 | Thioredoxin domain-containing | TXNDC5, |
| (SEQ ID NO: 113) | | | | protein 5 [118] | MUTED, |
| | | | | | MUTED- |
| | | | | | TXNDC5 |
| VVVTK(ac)YCDPDSYHR | 0.00 | −0.43 | 0.00 | Isoform 1 of Splicing factor | U2AF2 |
| (SEQ ID NO: 114) | | | | U2AF 65 kDa subunit [462] | |
| FGIAAK(ac)YQIDPDACFSAK | 0.00 | 0.47 | 0.00 | Voltage-dependent anion-selective | VDAC1 |
| (SEQ ID NO: 115) | | | | channel protein 1 [224] | |
| YK(ac)WCEYGLTFTEK | 0.00 | 0.59 | 0.00 | Isoform 2 of Voltage-dependent | VDAC2 |
| (SEQ ID NO: 116) | | | | anion-selective channel protein | |
| | | | | 2 [63] | |
| YK(ac)VCNYGLTFTQK | 0.00 | 0.40 | 0.00 | Isoform 1 of Voltage-dependent | VDAC3 |
| (SEQ ID NO: 117) | | | | anion-selective channel protein | |
| | | | | 3 [63] | |
| FANYIDK(ac)VR (SEQ ID NO: 118) | 0.00 | −0.65 | 0.00 | Vimentin [120] | VIM |
| ILLAELEQLK(ac)GQGK | 0.00 | −0.82 | 0.00 | Vimentin [139] | VIM |
| (SEQ ID NO: 119) | | | | | |
| LQDEIQNMK(ac)EEMAR | 0.00 | −0.72 | 0.00 | Vimentin [373] | VIM |
| (SEQ ID NO: 120) | | | | | |
| SK(ac)FADLSEAANR | 0.00 | −0.60 | 0.00 | Vimentin [294] | VIM |
| (SEQ ID NO: 121) | | | | | |
| TNEK(ac)VELQELNDR | 0.00 | −0.59 | 0.00 | Vimentin [104] | VIM |
| (SEQ ID NO: 122) | | | | | |
| ALQEK(ac)VEIK (SEQ ID NO: 123) | 0.00 | −0.46 | 0.00 | X-ray repair cross-complementing | XRCC5 |
| | | | | protein 5 [665] | |
| (ac)SIMSYNGGAIMAMK(ac)GK | NA | 1.00 | NA | RcPSMB3 (Fragment) [15] | — |
| (SEQ ID NO: 124) | | | | | |
| NLQK(ac)EVIHR (SEQ ID NO: 125) | NA | 0.54 | 0.00 | Isoform 1 of Peroxisomal acyl- | ACOX1 |
| | | | | coenzyme A oxidase 1 [504] | |
| LVDQSGPPHEPK(ac)FVYQAK | NA | −0.41 | 0.00 | Isoform 2 of Double-stranded RNA- | ADAR |
| (SEQ ID NO: 126) | | | | specific adenosine deaminase [757] | |
| HLSDSINQK(ac)HFEQAIER | NA | −0.39 | 0.00 | AFG3-like protein 2 [543] | AFG3L2 |
| (SEQ ID NO: 127) | | | | | |
| DAISGIGTDEK(ac)CLIEILASR | NA | 0.67 | NA | Annexin A6 [113] | ANXA6 |
| (SEQ ID NO: 128) | | | | | |
| DLMTDLK(ac)SEISGDLAR | NA | 0.42 | 0.00 | Annexin A6 [418] | ANXA6 |
| (SEQ ID NO: 129) | | | | | |
| GTVRPANDFNPDADAK(ac)ALR | NA | 0.44 | NA | Annexin A6 [370] | ANXA6 |
| (SEQ ID NO: 130) | | | | | |
| LENK(ac)MEGIGLK | NA | 0.67 | NA | PRA1 family protein 3 [151] | ARL6IP5 |
| (SEQ ID NO: 131) | | | | | |
| EQEHMINWVEK(ac)HVVQSISTQQEK | NA | 0.77 | 0.00 | ATP synthase subunit b | ATP5F1 |
| (SEQ ID NO: 132) | | | | mitochondrial [221] | |
| LNEQK(ac)LAQLEEAK | NA | 0.72 | 0.00 | ATP synthase subunit b | ATP5F1 |
| (SEQ ID NO: 133) | | | | mitochondrial [131] | |
| YGPFVADFADK(ac)LNEQK | NA | −0.45 | NA | ATP synthase subunit b | ATP5F1 |
| (SEQ ID NO: 134) | | | | mitochondrial [126] | |
| SCAEWVSLSK(ac)AR | NA | 0.75 | NA | Isoform 1 of ATP synthase | ATP5H |
| (SEQ ID NO: 135) | | | | subunit d mitochondrial [109] | |
| VNLQNNPGAMEHFHMK(ac)LFR | NA | 0.52 | 0.00 | B-cell receptor-associated | BCAP31 |
| (SEQ ID NO: 136) | | | | protein 31 [95] | |
| LTALDYHNPAGFNCK(ac)DETEFR | NA | 0.41 | 0.00 | UPF0568 protein C14orf166 [20] | C14orf166 |
| (SEQ ID NO: 137) | | | | | |
| IYFTDSSSK(ac)WQR | NA | −0.59 | NA | Isoform 1 of Adipocyte plasma | C20orf3 |
| (SEQ ID NO: 138) | | | | membrane-associated protein [220] | |
| TPELNLDQFHDK(ac)TPYTIMFGPDK | NA | −0.48 | 0.00 | cDNA FLJ55574 highly similar to | CANX |
| (SEQ ID NO: 139) | | | | Calnexin [217] | |
| YIAK(ac)QESVEER | NA | −0.42 | NA | Isoform 3 of Coiled-coil domain- | CCDC144A |
| (SEQ ID NO: 140) | | | | containing protein 144A [754] | |
| EDQK(ac)PVMDDQR | NA | −0.79 | NA | Isoform 1 of HLA class I | CD74 |
| (SEQ ID NO: 141) | | | | histocompatibility antigen gamma | |
| | | | | chain [14] | |
| SHLIDK(ac)GMLTSTTEDE | NA | −0.78 | NA | Isoform 1 of Phosphatidate | CDS2 |
| (SEQ ID NO: 142) | | | | cytidylyltransferase 2 [435] | |
| (ac)SVFGK(ac)LFGAGGGK | NA | −0.39 | 0.00 | Charged multivesicular body | CHMP4B |
| (SEQ ID NO: 143) | | | | protein 4b [6] | |
| STTELPLTVSYDK(ac)VSLGR | NA | −0.48 | NA | Isoform 1 of Cleft lip and | CLPTM1L |
| (SEQ ID NO: 144) | | | | palate transmembrane protein | |
| | | | | 1-like protein [243] | |
| DCDHADEQK(ac)CYSCGEFGHIQK | NA | −0.38 | 0.00 | Isoform 1 of Cellular nucleic | CNBP |
| (SEQ ID NO: 145) | | | | acid-binding protein [118] | |
| GPIGSIGPK(ac)GIPGEDGYR | NA | −0.80 | NA | Isoform 1 of Collagen alpha-3(VI) | COL6A3 |
| (SEQ ID NO: 146) | | | | chain [2052] | |
| YLLYGEK(ac)GTGK | NA | 0.46 | NA | 28S ribosomal protein S29 | DAP3 |
| (SEQ ID NO: 147) | | | | mitochondrial [130] | |
| NIDPK(ac)PCTPR | NA | 0.80 | 0.00 | Isoform 1 of DAZ-associated | DAZAP1 |
| (SEQ ID NO: 148) | | | | protein 1 [83] | |
| FNQK(ac)GEVYK | NA | 0.39 | 0.00 | Lipoamide acyltransferase component | DBT |
| (SEQ ID NO: 149) | | | | of branched-chain alpha-keto acid | |
| | | | | dehydrogenase complex | |
| | | | | mitochondrial [435] | |
| WVFK(ac)EEGVLR | NA | −0.53 | NA | Dolichyl-diphosphooligosaccharide-- | DDOST |
| (SEQ ID NO: 150) | | | | protein glycosyltransferase 48 kDa | |
| | | | | subunit [301] | |
| SK(ac)EITVR (SEQ ID NO: 151) | NA | −0.69 | NA | Probable ATP-dependent RNA | DDX5 |
| | | | | helicase DDX5 [80] | |
| K(ac)YEDICPSTHNMDVPNIK | NA | −0.56 | 0.00 | Eukaryotic translation | EIF5A2 |
| (SEQ ID NO: 152) | | | | initiation factor 5A-2 [68] | |
| AHPVFYQGTYSQALNDAK(ac)R | NA | −0.48 | NA | FAS-associated factor 2 [167] | FAF2 |
| (SEQ ID NO: 153) | | | | | |
| MPTPVIK(ac)AFGILK | NA | −0.55 | NA | Isoform Mitochondrial of Fumarate | FH |
| (SEQ ID NO: 154) | | | | hydratase mitochondrial [94] | |
| SNFK(ac)TCEESSFCK | NA | −0.42 | NA | Isoform 2 of Neutral alpha- | GANAB |
| (SEQ ID NO: 155) | | | | glucosidase AB [39] | |
| AVIWPQYVK(ac)DR | NA | 0.47 | NA | cDNA FLJ59630 highly similar to | GHITM |
| (SEQ ID NO: 156) | | | | Growth hormone-inducible | |
| | | | | transmembrane protein [124] | |
| EAALEPSMEK(ac)IFK | NA | −0.68 | 0.00 | cDNA FLJ59630 highly similar to | GHITM |
| (SEQ ID NO: 157) | | | | Growth hormone-inducible | |
| | | | | transmembrane protein [76] | |
| TAMK(ac)YNLGLDLR | NA | 0.48 | 0.00 | Glutamate dehydrogenase 1 | GLUD1 |
| (SEQ ID NO: 158) | | | | mitochondrial [527] | |
| NLDK(ac)EYLPIGGLAEFCK | NA | −0.46 | 0.00 | Aspartate aminotransferase | GOT2 |
| (SEQ ID NO: 159) | | | | mitochondrial [94] | |
| DSGNK(ac)PPGLLPR | NA | 0.47 | NA | Isoform 1 of Glutathione S- | GSTK1 |
| (SEQ ID NO: 160) | | | | transferase kappa 1 [54] | |
| GDASK(ac)EDIDTAMK | NA | 0.47 | 0.00 | Isoform 1 of Hydroxyacyl-coenzyme | HADH |
| (SEQ ID NO: 161) | | | | A dehydrogenase mitochondrial | |
| | | | | [241] | |
| GFGGK(ac)YGVER | NA | 0.77 | 0.00 | Hematopoietic lineage cell- | HCLS1 |
| (SEQ ID NO: 162) | | | | specific protein [123] | |
| YLENGK(ac)ETLQR | NA | −0.46 | 0.00 | Putative HLA class I histocompati- | HLA-H |
| (SEQ ID NO: 163) | | | | bility antigen alpha chain H | |
| | | | | [200] | |
| K(ac)HPDASVNFSEFSK | NA | −0.39 | 0.00 | Similar to nonhistone chromosomal | HMGB1P4 |
| (SEQ ID NO: 164) | | | | protein HMG-1 [30] | |
| GFAFVTFDDHDSVDK(ac)IVIQK | NA | −0.53 | 0.00 | Isoform A1-B of Heterogeneous | HNRNPA1 |
| (SEQ ID NO: 165) | | | | nuclear ribonucleoprotein A1 [161] | |
| IFVGGLSPDTPEEK(ac)IR | NA | −0.78 | NA | Isoform 1 of Heterogeneous nuclear | HNRNPD |
| (SEQ ID NO: 166) | | | | ribonucleoprotein D0 [197] | |
| ISK(ac)EVLAGR | NA | −0.75 | 0.00 | Isoform Short of Heterogeneous | HNRNPU |
| (SEQ ID NO: 167) | | | | nuclear ribonucleoprotein U [417] | |
| VTEK(ac)IPVR | NA | −0.76 | NA | Isoform Short of Heterogeneous | HNRNPU |
| (SEQ ID NO: 168) | | | | nuclear ribonucleoprotein U [324] | |
| MKENQK(ac)HIYYITGETK | NA | −0.46 | 0.00 | Isoform 2 of Heat shock protein | HSP90AA1 |
| (SEQ ID NO: 169) | | | | HSP 90-alpha [611] | |
| AFYK(ac)SFSK | NA | −1.00 | 0.00 | Endoplasmin [360] | HSP90B1 |
| (SEQ ID NO: 170) | | | | | |
| K(ac)TFEINPR | NA | −0.73 | NA | Endoplasmin [683] | HSP90B1 |
| (SEQ ID NO: 171) | | | | | |
| AVEEK(ac)IEWLESHQDADIEDFK | NA | −0.54 | 0.00 | 78 kDa glucose-regulated protein | HSPA5 |
| (SEQ ID NO: 172) | | | | [601] | |
| DVK(ac)FGADAR | NA | −0.66 | 0.00 | 60 kDa heat shock protein | HSPD1 |
| (SEQ ID NO: 173) | | | | mitochondrial [31] | |
| IGIEIIK(ac)R | NA | −0.42 | NA | 60 kDa heat shock protein | HSPD1 |
| (SEQ ID NO: 174) | | | | mitochondrial [469] | |
| TLNDELEIIEGMK(ac)FDR | NA | −0.45 | NA | 60 kDa heat shock protein | HSPD1 |
| (SEQ ID NO: 175) | | | | mitochondrial [218] | |
| TPVIVTLK(ac)ENER | NA | −0.54 | 0.00 | Hypoxia up-regulated protein | HYOU1 |
| (SEQ ID NO: 176) | | | | 1 [75] | |
| NTILK(ac)AYDGR | NA | −0.49 | NA | Isocitrate dehydrogenase [NADP] | IDH2 |
| (SEQ ID NO: 177) | | | | mitochondrial [256] | |
| HK(ac)DIDILIVR | NA | −1.00 | NA | Isocitrate dehydrogenase [NAD] | IDH3G |
| (SEQ ID NO: 178) | | | | subunit gamma mitochondrial [159] | |
| FEK(ac)EQQQLNWK | NA | −0.47 | NA | Isoform Long of Integrin beta-7 | ITGB7 |
| (SEQ ID NO: 179) | | | | [763] | |
| IGFGSFVDK(ac)TVLPFVSTVPSK | NA | −0.40 | NA | Isoform Long of Integrin beta-7 | ITGB7 |
| (SEQ ID NO: 180) | | | | [199] | |
| EVHK(ac)QVVESAYEVIK | NA | −0.67 | 0.00 | Isoform 1 of L-lactate | LDHA |
| (SEQ ID NO: 181) | | | | dehydrogenase A chain [232] | |
| DK(ac)TQYIFNNMVLK | NA | −0.45 | NA | Alpha-mannosidase 2 [191] | MAN2A1 |
| (SEQ ID NO: 182) | | | | | |
| VALEIFQHSK(ac)YK | NA | 0.64 | NA | Isoform 2 of 39S ribosomal | MRPL39 |
| (SEQ ID NO: 183) | | | | protein L39 mitochondrial | |
| | | | | [234] | |
| DELGDYLK(ac)PK | NA | −0.96 | NA | 39S ribosomal protein L46 | MRPL46 |
| (SEQ ID NO: 184) | | | | mitochondrial [265] | |
| DAAGSGDK(ac)PGADTGR | NA | 0.38 | NA | 39S ribosomal protein L53 | MRPL53 |
| (SEQ ID NO: 185) | | | | mitochondrial [105] | |
| VLK(ac)GNTAEGCVHETQEK | NA | −0.43 | NA | Isoform 1 of Myb-binding protein | MYBBP1A |
| (SEQ ID NO: 186) | | | | 1A [935] | |
| FWNK(ac)FLENK (SEQ ID NO: 187) | NA | 0.54 | 0.00 | NADH dehydrogenase [ubiquinone] | NDUFB6 |
| | | | | 1 beta subcomplex subunit 6 [49] | |
| TYGEIFEK(ac)FHPIR | NA | 1.00 | 0.00 | NADH dehydrogenase [ubiquinone] | NDUFC2 |
| (SEQ ID NO: 188) | | | | 1 subunit C2 [114] | |
| PGGPALWGDAFK(ac)R | NA | 0.77 | NA | Protein NipSnap homolog 3A [166] | NIPSNAP3A |
| (SEQ ID NO: 189) | | | | | |
| QGFNVVVESGAGEASK(ac)FSDDHYR | NA | 0.48 | 0.00 | NAD(P) transhydrogenase | NNT |
| (SEQ ID NO: 190) | | | | mitochondrial [100] | |
| ADK(ac)DYHFK (SEQ ID NO: 191) | NA | −0.60 | 0.00 | Isoform 2 of Nucleophosmin [27] | NPM1 |
| NYEHLFK(ac)VNDK | NA | 1.00 | NA | Mitochondrial import inner | PAM16 |
| (SEQ ID NO: 192) | | | | membrane translocase subunit | |
| | | | | TIM16 [82] | |
| VVVGK(ac)TFDSIVMDPK | NA | −0.80 | 0.00 | Protein disulfide-isomerase A4 | PDIA4 |
| (SEQ ID NO: 193) | | | | [533] | |
| NATVGQSVLNIK(ac)YK | NA | 0.51 | NA | Peroxisome biogenesis factor | PEX2 |
| (SEQ ID NO: 194) | | | | 2 [84] | |
| VLPSIVNEVLK(ac)SVVAK | NA | 1.00 | NA | Prohibitin-2 [142] | PHB2 |
| (SEQ ID NO: 195) | | | | | |
| GDGTGGK(ac)SIYGER | NA | −0.54 | 0.00 | Peptidyl-prolyl cis-trans | PPIB |
| (SEQ ID NO: 196) | | | | isomerase B [116] | |
| GLFIIDDK(ac)GILR | NA | −0.77 | 0.00 | Peroxiredoxin-1 [136] | PRDX1 |
| (SEQ ID NO: 197) | | | | | |
| KLPK(ac)NEPQNATGAPGR | NA | 0.38 | 0.00 | Ras-related protein Rab-5C [184] | RAB5C |
| (SEQ ID NO: 198) | | | | | |
| AGYNVK(ac)QLFR | NA | 0.45 | NA | Isoform 1 of Ras-related protein | RAB6A |
| (SEQ ID NO: 199) | | | | Rab-6A [164] | |
| FLNAENAQK(ac)FK | NA | −0.68 | 0.00 | Ran-specific GTPase-activating | RANBP1 |
| (SEQ ID NO: 200) | | | | protein [150] | |
| NCMTDLLAK(ac)LEAK | NA | 0.49 | NA | Receptor expression-enhancing | REEP5 |
| (SEQ ID NO: 201) | | | | protein 5 [25] | |
| ADINTK(ac)WAATR | NA | −0.50 | 0.00 | Ribosomal protein L14 variant [85] | RPL14 |
| (SEQ ID NO: 202) | | | | | |
| RPAVK(ac)QFHDSK | NA | −0.54 | 0.00 | 60S ribosomal protein L18a [143] | RPL18A |
| (SEQ ID NO: 203) | | | | | |
| AYGGSMCAK(ac)CVR | NA | 0.40 | NA | 60S ribosomal protein L34 [85] | RPL34 |
| (SEQ ID NO: 204) | | | | | |
| NVTLPAVFK(ac)APIRPDIVNFVHTNLR | NA | −0.48 | NA | 60S ribosomal protein L4 [29] | RPL4 |
| (SEQ ID NO: 205) | | | | | |
| AGVNTVTTLVENK(ac)K | NA | −0.42 | NA | 60S ribosomal protein L7a [150] | RPL7A, |
| (SEQ ID NO: 206) | | | | | SNORD24 |
| GIVK(ac)DIIHDPGR | NA | −0.54 | 0.00 | 60S ribosomal protein L8 [46] | RPL8 |
| (SEQ ID NO: 207) | | | | | |
| GHLENNPALEK(ac)LLPHIR | NA | −0.48 | 0.00 | 60S acidic ribosomal protein | RPLP0 |
| (SEQ ID NO: 208) | | | | P0 [77] | |
| FFTVK(ac)LPVALDPGAK | NA | −0.39 | NA | Dolichyl-diphosphooligosaccharide-- | RPN1 |
| (SEQ ID NO: 209) | | | | protein glycosyltransferase | |
| | | | | subunit 1 precursor [155] | |
| NVESYTK(ac)LGNPTR | NA | −0.88 | NA | Dolichyl-diphosphooligosaccharide-- | RPN1 |
| (SEQ ID NO: 210) | | | | protein glycosyltransferase | |
| | | | | subunit 1 precursor [226] | |
| DLEK(ac)WQNNLLPSR | NA | −0.46 | 0.00 | 40S ribosomal protein 515a [88] | RPS15A |
| (SEQ ID NO: 211) | | | | | |
| IQDK(ac)EGIPPDQQR | NA | −0.50 | 0.00 | Ubiquitin-40S ribosomal protein | RPS27A |
| (SEQ ID NO: 212) | | | | S27a [33] | |
| LIYDTK(ac)GR (SEQ ID NO: 213) | NA | −0.40 | NA | 40S ribosomal protein S4 X | RPS4X |
| | | | | isoform [106] | |
| NKEEAAEYAK(ac)LLAK | NA | 0.53 | NA | 40S ribosomal protein S6 [211] | RPS6 |
| (SEQ ID NO: 214) | | | | | |
| LIK(ac)VHLDK (SEQ ID NO: 215) | NA | −0.50 | 0.00 | 40S ribosomal protein S7 [155] | RPS7 |
| GYLTK(ac)EDLR (SEQ ID NO: 216) | NA | 1.00 | 0.00 | Protein S100-A10 [28] | S100A10 |
| IMK(ac)DLDQCR (SEQ ID NO: 217) | NA | 0.72 | 0.00 | Protein S100-A10 [57] | S100A10 |
| FLTK(ac)GDNNAVDDR | NA | 0.82 | NA | Putative signal peptidase complex | SEC11B |
| (SEQ ID NO: 218) | | | | catalytic subunit SEC11B [101] | |
| FLEVIKPFCVILPEIQK(ac)PER | NA | 0.49 | NA | cDNA FLJ59739 highly similar to | SEC61A1 |
| (SEQ ID NO: 219) | | | | Protein transport protein Sec61 | |
| | | | | subunit alpha isoform 1 [27] | |
| GLSSLLYGSIPK(ac)AAVR | NA | 0.44 | 0.00 | Tricarboxylate transport protein | SLC25A1 |
| (SEQ ID NO: 220) | | | | mitochondrial [97] | |
| LTEAKPVDK(ac)VK | NA | −0.50 | 0.00 | cDNA FLJ60124 highly similar to | SLC25A10 |
| (SEQ ID NO: 221) | | | | Mitochondrial dicarboxylate | |
| | | | | carrier [142] | |
| SLEK(ac)VCADLIR | NA | −0.39 | NA | 40S ribosomal protein S20 [34] | SNORD54, |
| (SEQ ID NO: 222) | | | | | RPS20 |
| EVQTNDLK(ac)EVVNK | NA | −0.60 | 0.00 | 40S ribosomal protein S3a [182] | SNORD73A, |
| (SEQ ID NO: 223) | | | | | RPS3A |
| (ac)SIGVPIK(ac) | NA | 0.40 | 0.00 | Small nuclear ribonucleoprotein Sm | SNRPD3 |
| VLHEAEGHIVTCETNTGEVYR | | | | D3 [8] | |
| (SEQ ID NO: 224) | | | | | |
| AVK(ac)HAEELER | NA | −1.00 | NA | Isoform 2 of Signal recognition | SRP68 |
| (SEQ ID NO: 225) | | | | particle 68 kDa protein [164] | |
| LSLDK(ac)VFR (SEQ ID NO: 226) | NA | −0.41 | 0.00 | Stomatin-like protein 2 [145] | STOML2 |
| HIK(ac)ENDYYTPTGEFR | NA | −0.44 | NA | Dolichyl-diphosphooligosaccharide-- | STT3A |
| (SEQ ID NO: 227) | | | | protein glycosyltransferase | |
| | | | | subunit STT3A [613] | |
| IGGSTDTGK(ac)HIK | NA | −1.00 | NA | Dolichyl-diphosphooligosaccharide-- | STT3A |
| (SEQ ID NO: 228) | | | | protein glycosyltransferase | |
| | | | | subunit STT3A [610] | |
| LFVGSIPK(ac)SK | NA | −0.46 | NA | Isoform 1 of Heterogeneous nuclear | SYNCRIP |
| (SEQ ID NO: 229) | | | | ribonucleoprotein Q [252] | |
| HYEVEILDAK(ac)TR | NA | 0.51 | NA | Isoform 1 of Trans-23-enoyl-CoA | TECR |
| (SEQ ID NO: 230) | | | | reductase [12] | |
| NAVQALIDK(ac)HQR | NA | −0.53 | NA | Isoform 2 of Transmembrane | TMEM68 |
| (SEQ ID NO: 231) | | | | protein 68 [240] | |
| HVFTTFYAK(ac)TK | NA | 0.52 | NA | Isoform 1 of Transmembrane | TMEM70 |
| (SEQ ID NO: 232) | | | | protein 70 mitochondrial [215] | |
| SFEEK(ac)VENLK (SEQ ID NO: 233) | NA | −0.51 | 0.00 | Isoform 1 of Tumor protein | TPD52 |
| | | | | D52 [180] | |
| SDCK(ac)EFSSEAR | NA | −1.00 | NA | Heat shock protein 75 kDa | TRAP1 |
| (SEQ ID NO: 234) | | | | mitochondrial [262] | |
| SVQK(ac)LLDAVDTYIPVPAR | NA | −0.74 | NA | elongation factor Tu mitochondrial | TUFM |
| (SEQ ID NO: 235) | | | | precursor [241] | |
| IGK(ac)VDCTQHYELCSGNQVR | NA | −0.44 | 0.00 | Thioredoxin domain-containing | TXNDC5, |
| (SEQ ID NO: 236) | | | | protein 5 [244] | MUTED, |
| | | | | | MUTED- |
| | | | | | TXNDC5 |
| NK(ac)TEDLEATSEHFK | NA | 0.70 | 0.00 | Vesicle-associated membrane | VAMP8 |
| (SEQ ID NO: 237) | | | | protein 8 [47] | |
| FLK(ac)FGMTPSK | NA | −0.89 | NA | Transitional endoplasmic reticulum | VCP |
| (SEQ ID NO: 238) | | | | ATPase [505] | |
| DIFNK(ac)GFGFGLVK | NA | 0.48 | NA | Isoform 2 of Voltage-dependent | VDAC2 |
| (SEQ ID NO: 239) | | | | anion-selective channel protein | |
| | | | | 2 [20] | |
| GFGFGLVK(ac)LDVK | NA | 0.40 | NA | Isoform 2 of Voltage-dependent | VDAC2 |
| (SEQ ID NO: 240) | | | | anion-selective channel protein | |
| | | | | 2 [28] | |
| FLEQQNK(ac)ILLAELEQLK | NA | −0.52 | 0.00 | Vimentin [129] | VIM |
| (SEQ ID NO: 241) | | | | | |
| KVESLQEEIAFLK(ac)K | NA | −0.68 | 0.00 | Vimentin [235] | VIM |
| (SEQ ID NO: 242) | | | | | |
| FPTAIFESK(ac)GFR | NA | 0.40 | NA | Isoform 4 of Histone-lysine N- | WHSC1L1 |
| (SEQ ID NO: 243) | | | | methyltransferase NSD3 [727] | |
| LHTGEK(ac)PYK (SEQ ID NO: 244) | NA | −0.59 | NA | Zinc finger and SCAN domain- | ZSCAN21 |
| | | | | containing protein 21 [414] | |
| KTPCGEGSK(ac)TWDR | −0.69 | −0.46 | 0.00 | 40S ribosomal protein S20 [75] | SNORD54, |
| (SEQ ID NO: 245) | | | | | RPS20 |
| K(ac)YSQFINFPIYVWSSK | −0.54 | −0.41 | 0.00 | Endoplasmin [270] | HSP90B1 |
| (SEQ ID NO: 246) | | | | | |
| PLISVYSEK(ac)GESSGK | −0.54 | −0.38 | NA | 60S ribosomal protein L4 [14] | RPL4 |
| (SEQ ID NO: 247) | | | | | |
| ALDLNLK(ac)HQILPK | −0.50 | 0.46 | 0.00 | Cytochrome b-c1 complex subunit | UQCRB |
| (SEQ ID NO: 248) | | | | 7 [72] | |
| IGQGYLIK(ac)DGK | −0.44 | −0.46 | 0.00 | 60S ribosomal protein L3 [294] | SNORD43, |
| (SEQ ID NO: 249) | | | | | RPL3 |
| VGIVGK(ac)YGTR | −0.44 | −0.56 | 0.00 | 60S ribosomal protein L37a [13] | RPL37A |
| (SEQ ID NO: 250) | | | | | |
| LYATMEK(ac)HK (SEQ ID NO: 251) | −0.43 | 0.38 | 0.00 | Histone acetyltransferase | EP300 |
| | | | | p300 [1590] | |
| TLQYK(ac)LLEPVLLLGK | −0.39 | −0.53 | 0.00 | 40S ribosomal protein S16 [50] | RPS16 |
| (SEQ ID NO: 252) | | | | | |
| FPHSAHQK(ac)YVR | −0.39 | −0.87 | 0.00 | Ribosomal protein L14 variant | RPL14 |
| (SEQ ID NO: 253) | | | | [71] | |
| YNCDK(ac)MICR (SEQ ID NO: 254) | 0.38 | −0.41 | 0.00 | Ubiquitin-605 ribosomal protein | UBA52 |
| | | | | L40 [93] | |
| SK(ac)SDPIMLLK | 0.39 | 0.52 | 0.00 | pyruvate dehydrogenase E1 | PDHA1 |
| (SEQ ID NO: 255) | | | | component subunit alpha somatic | |
| | | | | form mitochondrial isoform 2 | |
| | | | | precursor [351] | |
| NICSK(ac)YSVR (SEQ ID NO: 256) | 0.39 | −0.51 | 0.00 | Thioredoxin domain-containing | TXNDC5, |
| | | | | protein 5 [390] | MUTED, |
| | | | | | MUTED- |
| | | | | | TXNDC5 |
| NVLINK(ac)DIR (SEQ ID NO: 257) | 0.39 | −0.88 | 0.00 | Calreticulin [159] | CALR |
| YQQFK(ac)DFQR (SEQ ID NO: 258) | 0.39 | −0.38 | 0.00 | Isoform 2 of Spliceosome RNA | SNORD84, |
| | | | | helicase DDX39B [349] | DDX39B |
| IPK(ac)HLTDAYFK | 0.40 | −0.63 | NA | 60S ribosomal protein L6 [211] | RPL6 |
| (SEQ ID NO: 259) | | | | | |
| AGLQVYNK(ac)CWK | 0.41 | −0.45 | 0.00 | CD59 glycoprotein [63] | CD59 |
| (SEQ ID NO: 260) | | | | | |
| EVEEDEYK(ac)AFYK | 0.41 | −0.89 | 0.00 | Endoplasmin[ 356] | HSP90B1 |
| (SEQ ID NO: 261) | | | | | |
| AVLK(ac)FAAATGATPIAGR | 0.41 | −0.53 | 0.00 | Laminin receptor-like protein | SNORA6, |
| (SEQ ID NO: 262) | | | | LAMRL5 [89] | RPSAP15, |
| | | | | | SNORA62, |
| | | | | | RPSA |
| FVVEK(ac)AEQQK | 0.42 | 0.62 | 0.00 | Prohibitin [202] | PHB |
| (SEQ ID NO: 263) | | | | | |
| IQEVLK(ac)HAR (SEQ ID NO: 264) | 0.43 | 0.38 | 0.00 | 39S ribosomal protein L20 | MRPL20 |
| | | | | mitochondrial [26] | |
| GSK(ac)FYGPAGPYGIFAGR | 0.43 | −0.45 | NA | membrane-associated progesterone | PGRMC2 |
| (SEQ ID NO: 265) | | | | receptor component 2 [159] | |
| AK(ac)FEELNMDLFR | 0.44 | −0.52 | 0.00 | 78 kDa glucose-regulated protein | HSPA5 |
| (SEQ ID NO: 266) | | | | [326] | |
| NQVALNPQNTVFDAK(ac)R | 0.44 | 0.59 | 0.00 | Heat shock 70 kDa protein 1A/1B | HSPA1B, |
| (SEQ ID NO: 267) | | | | [71] | HSPA1A |
| DGLQNEK(ac)NIVSTPVK | 0.45 | 0.40 | 0.00 | Hydroxymethylglutaryl-CoA lyase | HMGCL |
| (SEQ ID NO: 268) | | | | mitochondrial [48] | |
| TLLIK(ac)TVETR | 0.45 | −0.54 | 0.00 | Vimentin [445] | VIM |
| (SEQ ID NO: 269) | | | | | |
| TKYEK(ac)SLYSMIK | 0.46 | 0.42 | 0.00 | Annexin A6 [299] | ANXA6 |
| (SEQ ID NO: 270) | | | | | |
| QNK(ac)LEQVEK (SEQ ID NO: 271) | 0.46 | 0.45 | 0.00 | ATP synthase subunit O | ATP50 |
| | | | | mitochondrial [54] | |
| ATVPK(ac)TEIR (SEQ ID NO: 272) | 0.47 | −0.44 | 0.00 | Isoform 1 of 40S ribosomal | RPS24 |
| | | | | protein S24 [37] | |
| VHVIFNYK(ac)GK | 0.48 | −0.58 | NA | Calreticulin [151] | CALR |
| (SEQ ID NO: 273) | | | | | |
| YDGK(ac)WEVEEMK | 0.48 | −0.49 | 0.00 | cDNA FLJ55574 highly similar to | CANX |
| (SEQ ID NO: 274) | | | | Calnexin [138] | |
| RMELK(ac)ADQLYK | 0.51 | 0.53 | NA | Pyruvate dehydrogenase E1 | PDHA2 |
| (SEQ ID NO: 275) | | | | component subunit alpha testis- | |
| | | | | specific form mitochondrial [75] | |
| VMEHFIK(ac)LYK | 0.54 | −0.66 | 0.00 | 78 kDa glucose-regulated | HSPA5 |
| (SEQ ID NO: 276) | | | | protein [268] | |
| (ac)AAK(ac)VFESIGK | 0.54 | 0.39 | NA | Prohibitin [4] | PHB |
| (SEQ ID NO: 277) | | | | | |
| GWLK(ac)SNVSDAVAQSTR | 0.54 | −0.41 | 0.00 | triosephosphate isomerase | TPI1, |
| (SEQ ID NO: 278) | | | | isoform 2 [231] | TPI1P1 |
| IVNSAQTGSFK(ac)QLTVK | 0.56 | 0.77 | 0.00 | ATP synthase subunit g | ATP5L |
| (SEQ ID NO: 279) | | | | mitochondrial [66] | |
| IFK(ac)SDGLR (SEQ ID NO: 280) | 0.56 | 0.48 | 0.00 | ADP/ATP translocase 1 [166] | SLC25A4 |
| FVLSSGK(ac)FYGDEEKDK | 0.58 | −0.51 | 0.00 | Calreticulin [55] | CALR |
| (SEQ ID NO: 281) | | | | | |
| AINEAYK(ac)EDYHK | 0.59 | 0.53 | 0.00 | Annexin A6 [478] | ANXA6 |
| (SEQ ID NO: 282) | | | | | |
| (ac)AEYLASIFGTEK(ac)DK | 0.60 | 0.60 | NA | Splicing factor U2AF 35 kDa | U2AF1 |
| (SEQ ID NO: 283) | | | | subunit [13] | |
| AISPDK(ac)DNFYFDVK | 0.61 | 0.40 | 0.00 | NAD(P) transhydrogenase | NNT |
| (SEQ ID NO: 284) | | | | mitochondrial [403] | |
| NDTSGEYK(ac)K (SEQ ID NO: 285) | 0.61 | 0.70 | NA | Annexin A6 [314] | ANXA6 |
| NFEDVAFDEK(ac)K | 0.62 | −0.41 | 0.00 | Protein disulfide-isomerase [385] | P4HB |
| (SEQ ID NO: 286) | | | | | |
| FK(ac)LITEDVQGK | 0.62 | −0.66 | NA | 40S ribosomal protein S3a [85] | SNORD73A, |
| (SEQ ID NO: 287) | | | | | RPS3A |
| EIFQNEK(ac)R (SEQ ID NO: 288) | 0.64 | 0.55 | 0.00 | NAD(P) transhydrogenase | NNT |
| | | | | mitochondrial [70] | |
| TCDLVGEK(ac)GK | 0.65 | −0.81 | NA | Cation-dependent mannose-6- | M6PR |
| (SEQ ID NO: 289) | | | | phosphate receptor [38] | |
| VPVPEDK(ac)YTAQVDAEEKEDVK | 0.66 | 0.50 | 0.00 | Isoform 1 of ATP synthase | ATP5H |
| (SEQ ID NO: 290) | | | | subunit d mitochondrial [85] | |
| ILMSK(ac)PVLSGGTGR | 0.67 | 0.65 | NA | cDNA FLJ55380 highly similar to | ZMYND8 |
| (SEQ ID NO: 291) | | | | Protein kinase C-binding protein | |
| | | | | 1 [440] | |
| SAYFAEK(ac)LYK | 0.67 | 0.62 | NA | annexin A4 [255] | ANXA4 |
| (SEQ ID NO: 292) | | | | | |
| YFLDK(ac)TLTSR | 0.68 | 0.50 | NA | [3-methyl-2-oxobutanoate | BCKDK |
| (SEQ ID NO: 293) | | | | dehydrogenase [lipoamide]] kinase | |
| | | | | mitochondrial [192] | |
| VLSK(ac)EFHLNESGDPSSK | 0.69 | −1.00 | 0.00 | Isoform 1 of Protein SET [154] | SET |
| (SEQ ID NO: 294) | | | | | |
| SEVDMLK(ac)IR (SEQ ID NO: 295) | 0.71 | 0.41 | 0.00 | Putative annexin A2-like | ANXA2P2 |
| | | | | protein [302] | |
| LVAENK(ac)FGK (SEQ ID NO: 296) | 0.74 | −0.43 | 0.00 | Isoform 1 of Hydroxyacyl-coenzyme | HADH |
| | | | | A dehydrogenase mitochondrial | |
| | | | | [301] | |
| THILLFLPK(ac)SVSDYDGK | 0.75 | −0.45 | 0.00 | Protein disulfide-isomerase [263] | P4HB |
| (SEQ ID NO: 297) | | | | | |
| NKPLFFADK(ac)LYK | 0.78 | 0.51 | 0.00 | Annexin A6 [607] | ANXA6 |
| (SEQ ID NO: 298) | | | | | |
| ISK(ac)EEAMR (SEQ ID NO: 299) | 0.84 | −0.48 | 0.00 | Isoform 1 of 60S ribosomal | RPL11 |
| | | | | protein L11 [159] | |
| AIFAGYK(ac)R (SEQ ID NO: 300) | 0.86 | 0.44 | NA | 60S ribosomal protein L35a [15] | RPL35A |
| AGLVDDFEK(ac)K | 0.86 | 0.66 | NA | Isoform 1 of ATP synthase | ATP5H |
| (SEQ ID NO: 301) | | | | subunit d mitochondrial [72] | |
| LLEQYK(ac)EESK | 0.89 | −0.57 | NA | Isoform Short of Heterogeneous | HNRNPU |
| (SEQ ID NO: 302) | | | | nuclear ribonucleoprotein U [651] | |
| VLVGK(ac)NFEDVAFDEK | 0.99 | −0.41 | 0.00 | Protein disulfide-isomerase [375] | P4HB |
| (SEQ ID NO: 303) | | | | | |
| LEMSYSK(ac)FK (SEQ ID NO: 304) | 1.00 | 0.48 | NA | Isoform 1 of UDP-N- | DPAGT1 |
| | | | | acetylglucosamine--dolichyl- | |
| | | | | phosphate N- | |
| | | | | acetylglucosaminephospho- | |
| | | | | transferase [320] | |
| PAQGAK(ac)YR (SEQ ID NO: 305) | 1.00 | 0.39 | NA | Annexin A6 [9] | ANXA6 |
| IFGSNK(ac)WTTEQQQR | 1.00 | 0.45 | NA | ADP-ribosylation factor-like | ARL6IP1 |
| (SEQ ID NO: 306) | | | | protein 6-interacting protein | |
| | | | | 1 [96] | |
| HVVQSISTQQEK(ac)ETIAK | 1.00 | 0.62 | 0.00 | ATP synthase subunit b | ATP5F1 |
| (SEQ ID NO: 307) | | | | mitochondrial [233] | |
| VPGK(ac)DTVTK (SEQ ID NO: 308) | 1.00 | 0.40 | 0.00 | Trifunctional enzyme subunit beta | HADHB |
| | | | | mitochondrial [272] | |
| PEPAK(ac)SAPAPK(ac)K | 1.00 | 0.58 | 0.00 | Histone H2B type 2-E [6:12] | HIST2H2BE |
| (SEQ ID NO: 309) | | | | | |
| K(ac)STGGK(ac)APR | 1.00 | 0.46 | 0.00 | Histone H3.2 [10:15] | HIST2H3D, |
| (SEQ ID NO: 310) | | | | | HIST2H3A, |
| | | | | | HIST2H3C |
| GK(ac)GGK(ac)GLGK | 1.00 | 0.49 | 0.00 | Histone H4 [6:9] | HIST2H4B, |
| (SEQ ID NO: 311) | | | | | HIST1H4C, |
| | | | | | HIST1H4J, |
| | | | | | HIST1H4D, |
| | | | | | HIST1H4A, |
| | | | | | HIST2H4A, |
| | | | | | HIST1H4I, |
| | | | | | HIST1H4K, |
| | | | | | HIST1H4E, |
| | | | | | HIST1H4L, |
| | | | | | HIST1H4F, |
| | | | | | HIST1H4H, |
| | | | | | HIST4H4, |
| | | | | | HIST1H4B |
| GLGK(ac)GGAK(ac)R | 1.00 | 0.49 | 0.00 | Histone H4 [13:17] | HIST2H4B, |
| (SEQ ID NO: 312) | | | | | HIST1H4C, |
| | | | | | HIST1H4J, |
| | | | | | HIST1H4D, |
| | | | | | HIST1H4A, |
| | | | | | HIST2H4A, |
| | | | | | HIST1H4I, |
| | | | | | HIST1H4K, |
| | | | | | HIST1H4E, |
| | | | | | HIST1H4L, |
| | | | | | HIST1H4F, |
| | | | | | HIST1H4H, |
| | | | | | HIST4H4, |
| | | | | | HIST1H4B |
| GLGK(ac)GGAKR | 1.00 | −0.62 | 0.00 | Histone H4 [13] | HIST2H4B, |
| (SEQ ID NO: 313) | | | | | HIST1H4C, |
| | | | | | HIST1H4J, |
| | | | | | HIST1H4D, |
| | | | | | HIST1H4A, |
| | | | | | HIST2H4A, |
| | | | | | HIST1H4I, |
| | | | | | HIST1H4K, |
| | | | | | HIST1H4E, |
| | | | | | HIST1H4L, |
| | | | | | HIST1H4F, |
| | | | | | HIST1H4H, |
| | | | | | HIST4H4, |
| | | | | | HIST1H4B |
| EK(ac)NLLHVTDTGVGMTR | 1.00 | −0.84 | 0.00 | Endoplasmin[ 142] | HSP90B1 |
| (SEQ ID NO: 314) | | | | | |
| KSDYIK(ac)LYVR | 1.00 | −0.70 | 0.00 | Endoplasmin [410] | HSP90B1 |
| (SEQ ID NO: 315) | | | | | |
| K(ac)SADTLWGIQK | 1.00 | 1.00 | 0.00 | Isoform 1 of L-lactate dehy- | LDHA |
| (SEQ ID NO: 316) | | | | drogenase A chain [318] | |
| IVADK(ac)DYSVTANSK | 1.00 | 0.51 | 0.00 | L-lactate dehydrogenase | LDHB |
| (SEQ ID NO: 317) | | | | B chain [82] | |
| VVDSMDALDK(ac)VVQER | 1.00 | 0.45 | 0.00 | Isoform 1 of 39S ribosomal | MRPL47 |
| (SEQ ID NO: 318) | | | | protein L47 mitochondrial [144] | |
| YSAK(ac)DYFFK (SEQ ID NO: 319) | 1.00 | 0.44 | NA | Alpha-soluble NSF attachment | NAPA |
| | | | | protein [203] | |
| GIVLEK(ac)VGVEAK | 1.00 | −0.55 | NA | 40S ribosomal protein S23 [54] | RPS23 |
| (SEQ ID NO: 320) | | | | | |
| DLQQYQSQAK(ac)QLFR | 1.00 | 0.46 | NA | Vesicle-trafficking protein | SEC22B |
| (SEQ ID NO: 321) | | | | SEC22b [38] | |
| LLASK(ac)SLLNR | 1.00 | −0.59 | NA | cDNA FLJ56303 highly similar to | SSR2 |
| (SEQ ID NO: 322) | | | | Translocon-associated protein | |
| | | | | subunit beta [46] | |
| TMSCSDK(ac)ILR | 0.00 | NA | −0.65 | Isoform 2 of Double-stranded RNA- | ADAR |
| (SEQ ID NO: 323) | | | | specific adenosine deaminase | |
| | | | | [1013] | |
| MVAAAK(ac)YAR (SEQ ID NO: 324) | 0.00 | 0.00 | −0.80 | Isoform Heart of ATP synthase | ATP5C1 |
| | | | | subunit gamma mitochondrial [55] | |
| FEDPK(ac)FEVIEKPQA | 0.00 | 0.00 | −0.50 | ATP synthase-coupling factor 6 | ATP5J |
| (SEQ ID NO: 325) | | | | mitochondrial [99] | |
| VQDGLSDIAEK(ac)FLK | 0.00 | 0.00 | 0.89 | Isoform 5 of Transcriptional | ATRX |
| (SEQ ID NO: 326) | | | | regulator ATRX [812] | |
| LNK(ac)PFLFDTK | 0.00 | 0.00 | 0.43 | cDNA FLJ55574 highly similar to | CANX |
| (SEQ ID NO: 327) | | | | Calnexin [172] | |
| AAEEVEAK(ac)FK | 0.00 | 0.00 | −0.42 | Coiled-coil-helix-coiled-coil- | CHCHD3 |
| (SEQ ID NO: 328) | | | | helix domain-containing | |
| | | | | protein 3 mitochondrial [173] | |
| SLLK(ac)PFCAALLK | 0.00 | NA | 0.48 | Isoform 1 of Cytoskeleton- | CKAP5 |
| (SEQ ID NO: 329) | | | | associated protein 5 [438] | |
| SMSTEGLMK(ac)FVDSK | 0.00 | NA | 0.76 | Citrate synthase mitochondrial | CS |
| (SEQ ID NO: 330) | | | | [459] | |
| YNIEK(ac)DIAAYIK | 0.00 | 0.00 | −0.45 | Dynein light chain 2 cytoplasmic | DYNLL2 |
| (SEQ ID NO: 331) | | | | [36] | |
| YLAEK(ac)YEWDVAEAR | 0.00 | NA | −0.40 | Elongation factor 2 [638] | EEF2 |
| (SEQ ID NO: 332) | | | | | |
| FK(ac)ILDAVVAQEPLHR | 0.00 | NA | −0.99 | 116 kDa U5 small nuclear | EFTUD2 |
| (SEQ ID NO: 333) | | | | ribonucleoprotein component [790] | |
| LEAMCFDGVK(ac)R | 0.00 | NA | −0.40 | S1 IF1 type family protein [56] | EIF1AXP1 |
| (SEQ ID NO: 334) | | | | | |
| LMCK(ac)PIFSK (SEQ ID NO: 335) | 0.00 | NA | 0.49 | Eukaryotic translation initiation | EIF2S3 |
| | | | | factor 2 subunit 3 [312] | |
| SNPSEVVEFTTCPDK(ac)PGIPVKPSVK | 0.00 | 0.00 | 0.40 | Isoform 2 of Fibronectin type-III | FNDC3A |
| (SEQ ID NO: 336) | | | | domain-containing protein 3A [507] | |
| EIAQEFK(ac)TDLR | 0.00 | NA | −0.49 | histone cluster 2 H3 pseudogene 2 | HIST2H3PS2 |
| (SEQ ID NO: 337) | | | | [80] | |
| APQCLGK(ac)FIEIAAR | 0.00 | 0.00 | 0.75 | Isoform Short of Heterogeneous | HNRNPU |
| (SEQ ID NO: 338) | | | | nuclear ribonucleoprotein U [546] | |
| MMVAGFK(ac)K (SEQ ID NO: 339) | 0.00 | NA | −0.40 | Isoform Short of Heterogeneous | HNRNPU |
| | | | | nuclear ribonucleoprotein U [524] | |
| FEYK(ac)YSFK (SEQ ID NO: 340) | 0.00 | NA | −0.52 | Protein ERGIC-53 [49] | LMAN1 |
| IINEVSK(ac)PLAHHIPVEK | 0.00 | 0.00 | −0.58 | cDNA FLJ77177 highly similar to | MANF |
| (SEQ ID NO: 341) | | | | Homo sapiens arginine-rich | |
| | | | | mutated in early stage tumors | |
| | | | | (ARMET) mRNA [97] | |
| FLNK(ac)LAEER (SEQ ID NO: 342) | 0.00 | NA | −0.44 | Matrin-3 [836] | MATR3, |
| | | | | | SNHG4 |
| VHLSQK(ac)YK (SEQ ID NO: 343) | 0.00 | NA | 0.40 | Matrin-3 [473] | MATR3, |
| | | | | | SNHG4 |
| INK(ac)ALDK (SEQ ID NO: 344) | 0.00 | NA | 0.43 | Isoform 1 of Myosin-9 [435] | MYH9 |
| FCLSK(ac)PGVYK | 0.00 | 0.00 | 0.59 | Nodal modulator 1 [630] | NOMO3, |
| (SEQ ID NO: 345) | | | | | NOMO1 |
| AQNDLIWNIK(ac)DELKK | 0.00 | NA | 0.83 | Poly [ADP-ribose] polymerase | PARP1 |
| (SEQ ID NO: 346) | | | | 1 [249] | |
| FPDENFK(ac)LK (SEQ ID NO: 347) | 0.00 | 0.00 | −1.00 | Peptidyl-prolyl cis-trans | PPIC |
| | | | | isomerase C [123] | |
| TNVPVK(ac)LFAR | 0.00 | 0.00 | 0.39 | cDNA FLJ77422 highly similar to | RALY |
| (SEQ ID NO: 348) | | | | Homo sapiens RNA binding protein | |
| | | | | autoantigenic (hnRNP-associated | |
| | | | | with lethal yellow homolog | |
| | | | | (mouse)) transcript variant | |
| | | | | 1 mRNA (Fragment) [165] | |
| GYAYVEFENPDEAEK(ac)ALK | 0.00 | 0.00 | −0.46 | Isoform 1 of RNA-binding protein | RNPS1 |
| (SEQ ID NO: 349) | | | | with serine-rich domain 1 [218] | |
| VTNLLMLK(ac)GK | 0.00 | NA | 0.83 | regulator of differentiation 1 | ROD1 |
| (SEQ ID NO: 350) | | | | isoform 2 [64] | |
| KIEISQHAK(ac)YTCSFCGK | 0.00 | NA | 0.41 | 60S ribosomal protein L37a [36] | RPL37A |
| (SEQ ID NO: 351) | | | | | |
| DTYIENEK(ac)LISGK | 0.00 | 0.00 | 0.77 | Dolichyl-diphosphooligosaccharide-- | RPN1 |
| (SEQ ID NO: 352) | | | | protein glycosyltransferase | |
| | | | | subunit 1 precursor [626] | |
| KDVHMPK(ac)HPELADK | 0.00 | 0.00 | 0.56 | 40S ribosomal protein S10 [31] | RPS10 |
| (SEQ ID NO: 353) | | | | | |
| TFEK(ac)SMMNLQTK | 0.00 | NA | 0.46 | DNA topoisomerase 1 [642] | TOP1 |
| (SEQ ID NO: 354) | | | | | |
| KQELLEALTK(ac)HFQD | 0.00 | 0.00 | −0.74 | X-ray repair cross-complementing | XRCC6 |
| (SEQ ID NO: 355) | | | | protein 6 [605] | |
| NFK(ac)SISK (SEQ ID NO: 356) | NA | NA | 0.44 | cDNA FLJ56425 highly similar to | ACADVL |
| | | | | Very-long-chain specific acyl- | |
| | | | | CoAdehydrogenase mitochondrial | |
| | | | | [681] | |
| TNFSNEK(ac)TISK | NA | NA | 0.84 | Acylphosphatase-2 [113] | ACYP2 |
| (SEQ ID NO: 357) | | | | | |
| K(ac)YGKSLYYYIQQDTK | NA | NA | 0.66 | Putative annexin A2-like | ANXA2P2 |
| (SEQ ID NO: 358) | | | | protein [310] | |
| RPEILK(ac)QEIK | NA | NA | 0.57 | Isoform 1 of AP-2 complex | AP2B1 |
| (SEQ ID NO: 359) | | | | subunit beta [318] | |
| ELQEMDKDDESLIK(ac)YK | NA | NA | 0.60 | Rho GDP-dissociation inhibitor | ARHGDIB |
| (SEQ ID NO: 360) | | | | 2 [47] | |
| NAYVWTLK(ac)GR | NA | NA | −1.00 | Actin-related protein 2/3 complex | ARPC1B, |
| (SEQ ID NO: 361) | | | | subunit 1B [82] | ARPC1A |
| VFDPQNDK(ac)PSKWWTCFVK | NA | NA | 0.45 | Actin-related protein 2/3 complex | ARPC3 |
| (SEQ ID NO: 362) | | | | subunit 3 [155] | |
| VLISFK(ac)ANDIEK | NA | NA | 0.42 | Isoform 2 of Actin-related | ARPC5 |
| (SEQ ID NO: 363) | | | | protein 2/3 complex subunit | |
| | | | | 5 [90] | |
| LAALPENPPAIDWAYYK(ac)ANVAK | NA | NA | 0.48 | Isoform 1 of ATP synthase | ATP5H |
| (SEQ ID NO: 364) | | | | subunit d mitochondrial | |
| | | | | [58] | |
| ELDPIQK(ac)LFVDK | NA | 0.00 | −0.48 | ATP synthase-coupling factor 6 | ATP5J |
| (SEQ ID NO: 365) | | | | mitochondrial [41] | |
| GHCPPAPAK(ac)PMHPENK(ac)LTNHGK | NA | NA | 0.63 | Isoform 1 of B-cell CLL/lymphoma | BCL9L |
| (SEQ ID NO: 366) | | | | 9-like protein [36:43] | |
| LVGELK(ac)LDR (SEQ ID NO: 367) | NA | NA | −0.52 | Bactericidal/permeability- | BPI |
| | | | | increasing protein [237] | |
| SNYK(ac)MMFVK (SEQ ID NO: 368) | NA | NA | −0.45 | Small acidic protein [174] | C11orf58 |
| K(ac)HDSGAADLER | NA | NA | 1.00 | Isoform 2 of Huntingtin- | C15orf63 |
| (SEQ ID NO: 369) | | | | interacting protein K [35] | |
| AELSK(ac)LVIVAK | NA | NA | 1.00 | UPF0556 protein C19orf10 [161] | C19orf10 |
| (SEQ ID NO: 370) | | | | | |
| LHSNYYK(ac)HK (SEQ ID NO: 371) | NA | NA | 0.52 | Uncharacterized protein C6orf125 | C6orf125 |
| | | | | [69] | |
| GQTLVVQFTVK(ac)HEQNIDCGGGYVK | NA | NA | −0.43 | Calreticulin [98] | CALR |
| (SEQ ID NO: 372) | | | | | |
| K(ac)VCGDSDKGFVVINQK | NA | NA | 0.67 | T-complex protein 1 subunit zeta | CCT6A |
| (SEQ ID NO: 373) | | | | [280] | |
| (ac)MEDYTK(ac)IEK | NA | NA | −0.51 | Isoform 2 of Cyclin-dependent | CDK1 |
| (SEQ ID NO: 374) | | | | kinase 1 [6] | |
| LK(ac)ADVFK (SEQ ID NO: 375) | NA | NA | −0.45 | Isoform 1 of Chromodomain- | CHD4 |
| | | | | helicase-DNA-binding | |
| | | | | protein 4 [959] | |
| (ac)SNMEK(ac)HLFNLK | NA | NA | 0.85 | Charged multivesicular body | CHMP1B |
| (SEQ ID NO: 376) | | | | protein 1b [6] | |
| GK(ac)YMLVR (SEQ ID NO: 377) | NA | NA | 1.00 | Carbohydrate sulfotransferase | CHST1 |
| | | | | 1 [296] | |
| FLDGNEMTLADCNLLPK(ac)LHIVK | NA | NA | 0.51 | Chloride intracellular channel | CLIC4 |
| (SEQ ID NO: 378) | | | | protein 4 [194] | |
| (ac)SSNECFK(ac)CGR | NA | 0.00 | −0.82 | Isoform 1 of Cellular nucleic | CNBP |
| (SEQ ID NO: 379) | | | | acid-binding protein [8] | |
| NLK(ac)NEITK (SEQ ID NO: 380) | NA | NA | 0.40 | Isoform 1 of Coatomer subunit | COPA |
| | | | | alpha [441] | |
| DHPLPEVAHVK(ac)HLSASQK | NA | 0.00 | −0.63 | Cytochrome c oxidase subunit 4 | COX4I1 |
| (SEQ ID NO: 381) | | | | isoform 1 mitochondrial [53] | |
| NLPFSVENK(ac)WSLLAK | NA | NA | −0.87 | Cytochrome c oxidase subunit 7C | COX7C |
| (SEQ ID NO: 382) | | | | mitochondrial [34] | |
| GLESTTLADK(ac)DGEIYCK | NA | NA | −0.67 | Cysteine and glycine-rich | CSRP1 |
| (SEQ ID NO: 383) | | | | protein 1 [161] | |
| HTENVAK(ac)FHCPHCDTVIAR | NA | NA | −0.66 | Transcriptional repressor CTCF | CTCF |
| (SEQ ID NO: 384) | | | | [436] | |
| LHYK(ac)HESWLLHR | NA | NA | −0.43 | Deoxycytidine kinase [211] | DCK |
| (SEQ ID NO: 385) | | | | | |
| QLNDVK(ac)TTVVYPATEK | NA | NA | 0.97 | Scavenger mRNA-decapping enzyme | DCPS |
| (SEQ ID NO: 386) | | | | DcpS [128] | |
| SHSAHFFEFLTK(ac)ELALGQDR | NA | NA | −0.44 | D-dopachrome decarboxylase [87] | DDT |
| (SEQ ID NO: 387) | | | | | |
| KDFIK(ac)TTVK (SEQ ID NO: 388) | NA | NA | 0.38 | Protein DEK [367] | DEK |
| LCMLYHPDK(ac)HRDPELK | NA | NA | −0.69 | Isoform 3 of DnaJ homolog | DNAJC11 |
| (SEQ ID NO: 389) | | | | subfamily C member 11 [45] | |
| LCK(ac)YIYAK (SEQ ID NO: 390) | NA | NA | 0.45 | Eukaryotic translation initiation | EIF3C, |
| | | | | factor 3 subunit C [553] | EIF3CL |
| KPEENPASK(ac)FSSASK | NA | NA | 0.53 | Eukaryotic translation initiation | EIF4B |
| (SEQ ID NO: 391) | | | | factor 4B [586] | |
| EENTSNESTDVTK(ac)GDSK(ac)NAK | NA | NA | 0.53 | Histone acetyltransferase p300 | EP300 |
| (SEQ ID NO: 392) | | | | [1542:1546] | |
| GLAPQNK(ac)PELQK | NA | NA | 0.69 | Isoform 2 of Protein FAM107B | FAM107B |
| (SEQ ID NO: 393) | | | | [225] | |
| VYISK(ac)CYR (SEQ ID NO: 394) | NA | NA | −0.85 | Isoform 1 of Regulator of micro- | FAM82A2 |
| | | | | tubule dynamics protein 3 [426] | |
| SFYYK(ac)LR (SEQ ID NO: 395) | NA | NA | −0.74 | Fatty acid synthase [2426] | FASN |
| AVSMDNSNK(ac)YTK | NA | NA | 0.45 | Forkhead box protein O3 [259] | FOXO3 |
| (SEQ ID NO: 396) | | | | | |
| FAFK(ac)EVEVQAK | NA | NA | 0.74 | Putative rRNA methyltransferase 3 | FTSJ3 |
| (SEQ ID NO: 397) | | | | [218] | |
| LVQTAELTK(ac)VFEIR | NA | NA | −0.80 | Glia maturation factor gamma [119] | GMFG |
| (SEQ ID NO: 398) | | | | | |
| LWNTLGVCK(ac)YTVQDESHSEWVSCVR | NA | NA | 0.54 | Guanine nucleotide-binding protein | GNB2L1 |
| (SEQ ID NO: 399) | | | | subunit beta-2-like 1 [139] | |
| EFTK(ac)LEEVLTNK | NA | NA | −0.60 | Glutathione S-transferase omega-1 | GSTO1 |
| (SEQ ID NO: 400) | | | | [152] | |
| VIIVVK(ac)DGPGFYTTR | NA | 0.00 | −0.70 | Trifunctional enzyme subunit alpha | HADHA |
| (SEQ ID NO: 401) | | | | mitochondrial [540] | |
| AK(ac)FESMAEEK | NA | NA | 1.00 | Hematopoietic lineage cell- | HCLS1 |
| (SEQ ID NO: 402) | | | | specific protein [241] | |
| ATAEEMTK(ac)YHSDEYIK | NA | NA | 0.78 | Histone deacetylase 2[161] | HDAC2 |
| (SEQ ID NO: 403) | | | | | |
| ESESVDK(ac)VMDQK | NA | NA | −0.53 | Isoform 1 of Heterogeneous nuclear | HNRNPD |
| (SEQ ID NO: 404) | | | | ribonucleoprotein D0 [153] | |
| VFITDDFHDMMPK(ac)YLNFVK | NA | NA | 0.77 | Endoplasmin [428] | HSP90B1 |
| (SEQ ID NO: 405) | | | | | |
| IQEIIEQLDVTTSEYEK(ac)EKLNER | NA | NA | 0.40 | 60 kDa heat shock protein | HSPD1 |
| (SEQ ID NO: 406) | | | | mitochondrial [387] | |
| TFYQMYDK(ac)GLVYR | NA | NA | −0.56 | Isoleucyl-tRNA synthetase | IARS2 |
| (SEQ ID NO: 407) | | | | mitochondrial [233] | |
| NVTAIQGPGGK(ac)WMIPSEAK | NA | NA | −0.44 | Isoform 1 of Isocitrate dehydro- | IDH3A |
| (SEQ ID NO: 408) | | | | genase [NAD] subunit alpha | |
| | | | | mitochondrial [77] | |
| KSEDTISK(ac)MNDFMR | NA | NA | −0.57 | Isoform 1 of Gamma-interferon- | IFI16 |
| (SEQ ID NO: 409) | | | | inducible protein 16 [451] | |
| SQSCETK(ac)LLDEK | NA | NA | −0.53 | Isoform 3 of Pro-interleukin- | IL16 |
| (SEQ ID NO: 410) | | | | 16 [277] | |
| KK(ac)FLCVTK (SEQ ID NO: 411) | NA | NA | 1.00 | Kelch repeat and BTB domain- | KBTBD8 |
| | | | | containing protein 8 [178] | |
| IQFK(ac)QDDGTGPEK | NA | NA | 0.61 | Isoform 1 of Far upstream | KHSRP |
| (SEQ ID NO: 412) | | | | element-binding protein | |
| | | | | 2 [359] | |
| K(ac)GPSVQKR (SEQ ID NO: 413) | NA | NA | 0.47 | Isoform 1 of Protein lin-28 | LIN28B |
| | | | | homolog B [240] | |
| LK(ac)AEKYR (SEQ ID NO: 414) | NA | NA | −1.00 | Isoform 1 of Leucine-rich | LRRIQ3 |
| | | | | repeat and IQ domain-containing | |
| | | | | protein 3 [557] | |
| FQAK(ac)LEHEYIQNFK | NA | NA | −1.00 | Microtubule-associated protein | MAPRE1 |
| (SEQ ID NO: 415) | | | | RP/EB family member 1 [66] | |
| NVQQTVSAK(ac)GPPEK(ac)R | NA | NA | 0.49 | Mediator of RNA polymerase II | MED6 |
| (SEQ ID NO: 416) | | | | transcription subunit 6 [243:248] | |
| YK(ac)VEYPIMYSTDPENGHIFNCIQR | NA | NA | −0.59 | Microsomal glutathione S- | MGST3 |
| (SEQ ID NO: 417) | | | | transferase 3 [37] | |
| TPSVGK(ac)AMDTPKPAGGDEK | NA | NA | −0.86 | Isoform Long of Antigen | MKI67 |
| (SEQ ID NO: 418) | | | | KI-67 [2264] | |
| IIWHK(ac)FK (SEQ ID NO: 419) | NA | NA | −0.73 | Isoform 1 of 39S ribosomal | MRPL47 |
| | | | | protein L47 mitochondrial | |
| | | | | [180] | |
| FK(ac)ECLDK (SEQ ID NO: 420) | NA | NA | 0.60 | Isoform 2 of N-alpha- | NAA15 |
| | | | | acetyltransferase 15 NatA | |
| | | | | auxiliary subunit [307] | |
| (ac)AQLGK(ac)LLK | NA | NA | 0.70 | NEDD8-activating enzyme E1 | NAE1 |
| (SEQ ID NO: 421) | | | | regulatory subunit [6] | |
| SELEFK(ac)FNR (SEQ ID NO: 422) | NA | NA | 0.53 | Sialic acid synthase [61] | NANS |
| YTEQITNEK(ac)LAMVK | NA | NA | −0.40 | NADH dehydrogenase [ubiquinone] 1 | NDUFA5 |
| (SEQ ID NO: 423) | | | | alpha subcomplex subunit 5 [55] | |
| YHVSEK(ac)PYGIVEK | NA | NA | −1.00 | NADH dehydrogenase [ubiquinone] 1 | NDUFB6 |
| (SEQ ID NO: 424) | | | | beta subcomplex subunit 6 [93] | |
| CHAFEK(ac)EWIECAHGIGYTR | NA | NA | −0.63 | NADH dehydrogenase [ubiquinone] | NDUFS5 |
| (SEQ ID NO: 425) | | | | iron-sulfur protein 5 [38] | |
| DVTK(ac)AMDEK (SEQ ID NO: 426) | NA | NA | 0.53 | Isoform 2 of 6-phosphofructokinase | PFKM |
| | | | | muscle type [329] | |
| EGVHGGLINK(ac)K | NA | NA | 0.58 | Profilin-1 [126] | PFN1 |
| (SEQ ID NO: 427) | | | | | |
| TVSK(ac)VDDFLANEAK | NA | NA | 0.74 | 6-phosphogluconate dehydrogenase | PGD |
| (SEQ ID NO: 428) | | | | decarboxylating [38] | |
| ITLPVDFVTADK(ac)FDENAK | NA | NA | 0.60 | Phosphoglycerate kinase 1 [291] | PGK1 |
| (SEQ ID NO: 429) | | | | | |
| NVTGHYISPFHDIPLK(ac)VNSK | NA | NA | 0.91 | Isoform 1 of Inorganic | PPA2 |
| (SEQ ID NO: 430) | | | | pyrophosphatase 2 | |
| | | | | mitochondrial [69] | |
| FEDENFHYK(ac)HDR | NA | NA | 0.46 | Peptidyl-prolyl cis-trans | PPID |
| (SEQ ID NO: 431) | | | | isomerase D [111] | |
| HSPEDPEK(ac)YSCFALFVK | NA | NA | −0.41 | Isoform 1 of DNA-dependent protein | PRKDC |
| (SEQ ID NO: 432) | | | | kinase catalytic subunit [700] | |
| MLK(ac)QMLFR (SEQ ID NO: 433) | NA | NA | −0.47 | Proteasome subunit beta type-7 | PSMB7 |
| | | | | [127] | |
| IMK(ac)SEVLR (SEQ ID NO: 434) | NA | NA | 0.41 | 26S protease regulatory subunit | PSMC3 |
| | | | | 6A [56] | |
| TTHLIAK(ac)EEMIHNLQ | NA | NA | −0.38 | 26S proteasome non-ATPase | PSMD12 |
| (SEQ ID NO: 435) | | | | regulatory subunit 12 [448] | |
| VAHLALK(ac)HR (SEQ ID NO: 436) | NA | NA | −0.57 | Isoform Rpn10A of 26S proteasome | PSMD4 |
| | | | | non-ATPase regulatory subunit | |
| | | | | 4 [9]8 | |
| (ac)SDAAVDTSSEITTK(ac)DLK | NA | NA | 0.61 | Isoform 2 of Prothymosin alpha | PTMA, |
| (SEQ ID NO: 437) | | | | [15] | MIR1244-3, |
| | | | | | MIR1244-2, |
| | | | | | MIR1244-1 |
| HMVMQK(ac)LLR (SEQ ID NO: 438) | NA | NA | 0.71 | Isoform 1 of Poly(U)-binding- | PUF60 |
| | | | | splicing factor PUF60 [454] | |
| AFQLK(ac)SR (SEQ ID NO: 439) | NA | NA | −0.89 | ribonucleoprotein PTB-binding | RAVER1 |
| | | | | 1 [579] | |
| IMQK(ac)YGFR (SEQ ID NO: 440) | NA | NA | −0.50 | Splicing factor 45 [245] | RBM17 |
| GNLYSFGCPEYGQLGHNSDGK(ac)FIAR | NA | NA | 0.75 | Protein RCC2 [293] | RCC2 |
| (SEQ ID NO: 441) | | | | | |
| WTPEVK(ac)HFCPNVPIILVGNK | NA | NA | −0.39 | Rho-related GTP-binding protein | RHOC |
| (SEQ ID NO: 442) | | | | RhoC [104] | |
| AYHLQK(ac)STCGK | NA | 0.00 | 0.42 | 60S ribosomal protein L37 [31] | RPL37 |
| (SEQ ID NO: 443) | | | | | |
| NVYK(ac)FELDTSER | NA | 0.00 | 0.54 | Dolichyl-diphosphooligosaccharide-- | RPN2 |
| (SEQ ID NO: 444) | | | | protein glycosyltransferase | |
| | | | | subunit 2 [460] | |
| VETFSGVYK(ac)K | NA | NA | −0.78 | 40S ribosomal protein S7 [178] | RPS7 |
| (SEQ ID NO: 445) | | | | | |
| MLEK(ac)DVCEK (SEQ ID NO: 446) | NA | NA | 0.62 | Isoform 1 of Protein RUFY3 [382] | RUFY3 |
| VEWAK(ac)FQER (SEQ ID NO: 447) | NA | NA | −0.55 | Splicing factor 3A subunit | SF3A1 |
| | | | | 1 [251] | |
| LNPK(ac)TGLIDYNQLALTAR | NA | NA | −0.41 | Serine hydroxymethyltransferase | SHMT2 |
| (SEQ ID NO: 448) | | | | mitochondrial [200] | |
| GNTPK(ac)YGLIFHSTFIGR | NA | NA | −0.41 | Nucleolar protein 56 [347] | SNORD110, |
| (SEQ ID NO: 449) | | | | | SNORD86, |
| | | | | | NOP56 |
| HHAAYVNNLNVTEEK(ac)YQEALAK | NA | NA | −0.67 | Superoxide dismutase [Mn] | SOD2 |
| (SEQ ID NO: 450) | | | | mitochondrial [68] | |
| TTTCSK(ac)LAYYYQR | NA | NA | 0.40 | Signal recognition particle 54 kDa | SRP54 |
| (SEQ ID NO: 451) | | | | protein [120] | |
| DK(ac)WLCPLSGK | NA | NA | 0.42 | Isoform 2 of Serrate RNA effector | SRRT |
| (SEQ ID NO: 452) | | | | molecule homolog [712] | |
| IIEDQQESLNK(ac)WK | NA | NA | 0.57 | Lupus La protein [328] | SSB |
| (SEQ ID NO: 453) | | | | | |
| IDYGEYMDK(ac)NNVR | NA | NA | −0.52 | Single-stranded DNA-binding | SSBP1 |
| (SEQ ID NO: 454) | | | | protein mitochondrial [122] | |
| GEDFPANNIVK(ac)FLVGFTNK | NA | NA | −0.88 | Isoform 1 of Translocon-associated | SSR1 |
| (SEQ ID NO: 455) | | | | protein subunit alpha [102] | |
| DK(ac)LAQQQAAAAAAAAAAASQQGSAK | NA | NA | 0.86 | F-box-like/WD repeat-containing | TBL1XR1 |
| (SEQ ID NO: 456) | | | | protein TBL1XR1 [102] | |
| IAVEAQNK(ac)YER | NA | NA | 0.63 | Nucleoprotein TPR [1081] | TPR |
| (SEQ ID NO: 457) | | | | | |
| SQNTK(ac)ISTQLDFASK | NA | NA | −0.45 | Nucleoprotein TPR [713] | TPR |
| (SEQ ID NO: 458) | | | | | |
| GK(ac)LEEQRPER | NA | NA | −0.42 | Tumor protein translationally- | TPT1 |
| (SEQ ID NO: 459) | | | | controlled 1 [102] | |
| QPAENVNQYLTDPK(ac)FVER | NA | NA | 0.44 | cDNA FLJ40024 fis clone | UBA1 |
| (SEQ ID NO: 460) | | | | STOMA2007745 highly similar to | |
| | | | | UBIQUITIN-ACTIVATING ENZYME E1 | |
| | | | | [119] | |
| YPPPYK(ac)HFWTAES | NA | NA | −0.50 | Isoform 2 of Ubiquitin-associated | UBAP2L |
| (SEQ ID NO: 461) | | | | protein 2-like [976] | |
| IYHPNVDK(ac)LGR | NA | NA | −0.70 | Ubiquitin-conjugating enzyme E2 N | UBE2N |
| (SEQ ID NO: 462) | | | | [82] | |
| ACK(ac)LAIR (SEQ ID NO: 463) | NA | NA | −0.58 | Transitional endoplasmic reticulum | VCP |
| | | | | ATPase [696] | |
| (ac)AVPPTYADLGK(ac)SAR | NA | 0.00 | −0.78 | Voltage-dependent anion-selective | VDAC1 |
| (SEQ ID NO: 464) | | | | channel protein 1 [12] | |
| GYGFGLIK(ac)LDLK | NA | 0.00 | −0.57 | Voltage-dependent anion-selective | VDAC1 |
| (SEQ ID NO: 465) | | | | channel protein 1 [28] | |
| LTLSALLDGK(ac)NVNAGGHK | NA | 0.00 | −0.66 | Voltage-dependent anion-selective | VDAC1 |
| (SEQ ID NO: 466) | | | | channel protein 1 [266] | |
| DVFNK(ac)GYGFGMVK | NA | NA | −0.73 | Isoform 1 of Voltage-dependent | VDAC3 |
| (SEQ ID NO: 467) | | | | anion-selective channel | |
| | | | | protein 3 [20] | |
| FGIAAK(ac)YMLDCR | NA | NA | 0.43 | Isoform 1 of Voltage-dependent | VDAC3 |
| (SEQ ID NO: 468) | | | | anion-selective channel protein 3 | |
| | | | | [224] | |
| LTLSALIDGK(ac)NFSAGGHK | NA | NA | −0.71 | Isoform 1 of Voltage-dependent | VDAC3 |
| (SEQ ID NO: 469) | | | | anion-selective channel protein 3 | |
| | | | | [266] | |
| SGK(ac)FMNPTDQAR | NA | NA | −0.46 | WW domain-binding protein 11 [13] | WBP11 |
| (SEQ ID NO: 470) | | | | | |
| QLGSILK(ac)TNVR | NA | NA | 0.93 | Exportin-1 [693] | XPO1 |
| (SEQ ID NO: 471) | | | | | |
| AIVEK(ac)LR (SEQ ID NO: 472) | NA | NA | 1.00 | X-ray repair cross-complementing | XRCC6 |
| | | | | protein 6 [468] | |
| CPICEK(ac)VIQGAGK | NA | NA | −0.44 | Zinc finger and BTB domain- | ZBTB7A |
| (SEQ ID NO: 473) | | | | containing protein 7A [389] | |
| SIDFPLTK(ac)VYVVEGSK | NA | NA | 0.61 | CAAX prenyl protease 1 homolog | ZMPSTE24 |
| (SEQ ID NO: 474) | | | | [251] | |
| IIEK(ac)DVMEGVTVDDHMMK | NA | NA | 0.40 | cDNA FLJ50345 highly similar to | ZNF326 |
| (SEQ ID NO: 475) | | | | Zinc finger protein 326 [298] | |
| LVSEK(ac)LASYQAAR | −0.82 | 0.00 | −0.56 | Isoform 2 of Myc proto-oncogene | MYC |
| (SEQ ID NO: 476) | | | | protein [163] | |
| SFDK(ac)NLYR (SEQ ID NO: 477) | −0.55 | 0.00 | −0.47 | DNA replication licensing factor | MCM4 |
| | | | | MCM4 [220] | |
| LVDTGDK(ac)FR (SEQ ID NO: 478) | −0.46 | NA | −0.42 | Isoform Long of Spectrin beta | SPTBN1 |
| | | | | chain brain 1 [1913] | |
| FGIAAK(ac)YQLDPTASISAK | −0.40 | 0.00 | −0.73 | Isoform 2 of Voltage-dependent | VDAC2 |
| (SEQ ID NO: 479) | | | | anion-selective channel protein 2 | |
| | | | | [224] | |
| IK(ac)EKYIDQEELNK | 0.39 | NA | 0.45 | Isoform 2 of Heat shock protein | HSP90AA1 |
| (SEQ ID NO: 480) | | | | HSP 90-alpha [403] | |
| SYAEELAK(ac)HGMK | 0.39 | 0.00 | 0.57 | Estradiol 17-beta-dehydrogenase 12 | HSD17B12 |
| (SEQ ID NO: 481) | | | | [72] | |
| EHLLK(ac)YEYQPFAGK | 0.47 | NA | 0.45 | Isoform 1 of WD repeat-containing | WDR1 |
| (SEQ ID NO: 482) | | | | protein 1 [95] | |
| GVHPK(ac)FPEGGK | 0.48 | NA | −0.48 | NADH-cytochrome b5 reductase 1 | CYB5R1 |
| (SEQ ID NO: 483) | | | | [124] | |
| LAK(ac)LSDGVAVLK | 0.61 | 0.00 | −0.42 | 60 kDa heat shock protein | HSPD1 |
| (SEQ ID NO: 484) | | | | mitochondrial [396] | |
| LTLDK(ac)LDVK (SEQ ID NO: 485) | 0.65 | NA | −0.39 | Phosphoglycerate kinase 1 [11] | PGK1 |
| DVTLQK(ac)QCVPFR | 0.71 | NA | −0.39 | 60S ribosomal protein L17 [55] | RPL17 |
| (SEQ ID NO: 486) | | | | | |
| FVATGHGK(ac)YEK | 0.75 | NA | 1.00 | Lymphocyte-specific | LOC100133284, |
| (SEQ ID NO: 487) | | | | protein 1 [327] | LSP1 |
| VAILK(ac)ANLR (SEQ ID NO: 488) | 0.80 | 0.00 | 1.00 | Transitional endoplasmic reticulum | VCP |
| | | | | ATPase [658] | |
| IVQK(ac)HPHTGDTKEEK | 1.00 | 0.00 | 0.83 | Death-associated protein 1 [29] | DAP |
| (SEQ ID NO: 489) | | | | | |
| GSK(ac)K(ac)AVTK | 1.00 | 0.00 | 0.44 | Histone H2B type 2-E [16:17] | HIST2H2BE |
| (SEQ ID NO: 490) | | | | | |
| PKPYDPPGEK(ac)MVAAK | −1.00 | NA | NA | Cyclic AMP-dependent transcription | ATF4 |
| (SEQ ID NO: 491) | | | | factor ATF-4 [267] | |
| RPPQTIK(ac)SVR | −1.00 | NA | NA | GDP-D-glucose phosphorylase | C15orf58 |
| (SEQ ID NO: 492) | | | | C15orf58 [113] | |
| STPAITLESPDIK(ac)YPLR | −1.00 | 0.00 | NA | Isoform 1 of NADH-cytochrome b5 | CYB5R3 |
| (SEQ ID NO: 493) | | | | reductase 3 [42] | |
| HGMK(ac)VVLISR | −1.00 | NA | NA | Estradiol 17-beta-dehydrogenase | HSD17B12 |
| (SEQ ID NO: 494) | | | | 12 [76] | |
| LALQK(ac)QLTK (SEQ ID NO: 495) | −1.00 | NA | NA | Isoform 2 of Zinc finger | ZNF385C |
| | | | | protein 385C [103] | |
| NK(ac)YGDAFIR (SEQ ID NO: 496) | −0.93 | NA | NA | U6 snRNA-associated Sm-like | LSM6 |
| | | | | protein LSm6 [59] | |
| AK(ac)HDELTYF (SEQ ID NO: 497) | −0.88 | NA | 0.00 | Cystatin-B [91] | CSTB |
| NK(ac)WFFQK (SEQ ID NO: 498) | −0.86 | 0.00 | NA | 60S ribosomal protein L27 [128] | RPL27 |
| LMK(ac)YGDSLYR | −0.78 | NA | NA | Nucleolar GTP-binding protein | GTPBP4 |
| (SEQ ID NO: 499) | | | | 1 [118] | |
| LPSSTK(ac)SGWPR | −0.78 | NA | 0.00 | cDNA FLJ75110 highly similar to | PHF15 |
| (SEQ ID NO: 500) | | | | Homo sapiens PHD finger protein | |
| | | | | 15 (PHF15) mRNA [38] | |
| TLAMDVMK(ac)PR | −0.76 | NA | NA | H(+)/CI(−) exchange transporter | CLCN5 |
| (SEQ ID NO: 501) | | | | 5 [587] | |
| SAQEAYK(ac)SYIR | −0.74 | NA | NA | ATP-dependent RNA helicase DDX18 | DDX18 |
| (SEQ ID NO: 502) | | | | [584] | |
| AEK(ac)DEPGAWEETFK | −0.71 | NA | NA | Isoform 2 of Neutral alpha- | GANAB |
| (SEQ ID NO: 503) | | | | glucosidase AB [242] | |
| FSPDDK(ac)YSR (SEQ ID NO: 504) | −0.68 | NA | 0.00 | H/ACA ribonucleoprotein complex | NOP10 |
| | | | | subunit 3 [40] | |
| LAAIVAK(ac)QVLLGR | −0.67 | 0.00 | NA | 60S ribosomal protein L13a [25] | SNORD32A, |
| (SEQ ID NO: 505) | | | | | RPL13A, |
| | | | | | SNORD33, |
| | | | | | SNORD34 |
| IDK(ac)PILK (SEQ ID NO: 506) | −0.65 | NA | 0.00 | 60S ribosomal protein L8 [177] | RPL8 |
| LVPLK(ac)ETIK (SEQ ID NO: 507) | −0.64 | NA | NA | ATP synthase subunit beta | ATP5B |
| | | | | mitochondrial [485] | |
| (ac)AEPVSPLK(ac)HFVLAK | −0.63 | NA | NA | Mitochondrial transmembrane | MFN1 |
| (SEQ ID NO: 508) | | | | GTPase FZO-2 [37] | |
| EEAIK(ac)FSEEQR | −0.60 | NA | 0.00 | X-ray repair cross-complementing | XRCC5 |
| (SEQ ID NO: 509) | | | | protein 5 [648] | |
| K(ac)EELTLEGIR | −0.60 | 0.00 | NA | Eukaryotic initiation factor | EIF4A1 |
| (SEQ ID NO: 510) | | | | 4A-I [238] | |
| TLFVK(ac)GLSEDTTEETLK | −0.59 | NA | NA | cDNA FLJ45706 fis clone | NCL |
| (SEQ ID NO: 511) | | | | FEBRA2028457 highly similar to | |
| | | | | Nucleolin [470] | |
| LIAGNK(ac)PVSFLTAQQLQQLQQQGQATQVR | −0.58 | 0.00 | 0.00 | Isoform 2 of Nuclear factor | NFRKB |
| (SEQ ID NO: 512) | | | | related to kappa-B-binding | |
| | | | | protein [1262] | |
| ALK(ac)YLVMDEADR | −0.58 | NA | NA | Probable ATP-dependent RNA | DDX47 |
| (SEQ ID NO: 513) | | | | helicase DDX47 [169] | |
| LLLFK(ac)TFSR (SEQ ID NO: 514) | −0.57 | 0.00 | NA | Heterogeneous nuclear | HNRNPUL2 |
| | | | | ribonucleoprotein U-like | |
| | | | | protein 2 [553] | |
| VLGTVK(ac)WFNVR | −0.57 | NA | NA | Isoform 1 of DNA-binding | CSDA |
| (SEQ ID NO: 515) | | | | protein A [96] | |
| TLK(ac)ALEYVFK | −0.57 | 0.00 | NA | Isoform 1 of Dedicator of | DOCK2 |
| (SEQ ID NO: 516) | | | | cytokinesis protein 2 [738] | |
| LMEK(ac)EINGSK | −0.56 | NA | NA | Atlastin-3 [309] | ATL3 |
| (SEQ ID NO: 517) | | | | | |
| AVDCLLDSK(ac)WAK | −0.51 | 0.00 | NA | Translocation protein SEC62 [60] | SEC62 |
| (SEQ ID NO: 518) | | | | | |
| GHK(ac)FHHTIGGSR | −0.51 | NA | 0.00 | 60S ribosomal protein L15 [179] | RPL15 |
| (SEQ ID NO: 519) | | | | | |
| VYVLK(ac)FK (SEQ ID NO: 520) | −0.50 | NA | NA | Proteasomal ubiquitin receptor | ADRM1 |
| | | | | ADRM1 [97] | |
| LSFISVGNK(ac)FK | −0.49 | NA | NA | Isoform 2 of Myosin-VI [646] | MYO6 |
| (SEQ ID NO: 521) | | | | | |
| VITEEEK(ac)NFK | −0.46 | 0.00 | 0.00 | 60S ribosomal protein L13 [174] | RPL13 |
| (SEQ ID NO: 522) | | | | | |
| TLVLSDK(ac)HSPQK | −0.46 | 0.00 | 0.00 | Isoform 1 of Histone-lysine N- | MLL3 |
| (SEQ ID NO: 523) | | | | methyltransferase MLL3 [2809] | |
| SQFLK(ac)YIEK (SEQ ID NO: 524) | −0.46 | NA | NA | DNA replication licensing factor | MCM2 |
| | | | | MCM2 [534] | |
| SEIINSK(ac)NFDR | −0.45 | NA | 0.00 | Transmembrane protein 43 [159] | TMEM43 |
| (SEQ ID NO: 525) | | | | | |
| MQK(ac)EITALAPSTMK | −0.44 | NA | NA | Actin aortic smooth muscle [317] | ACTA2 |
| (SEQ ID NO: 526) | | | | | |
| ETGYTYILPK(ac)NVLK | −0.44 | NA | NA | Pre-mRNA-processing-splicing | PRPF8 |
| (SEQ ID NO: 527) | | | | factor 8 [2108] | |
| LLNK(ac)HGIK (SEQ ID NO: 528) | −0.43 | NA | NA | Ribosomal L1 domain-containing | RSL1D1 |
| | | | | protein 1 [120] | |
| K(ac)VLFTCFK (SEQ ID NO: 529) | −0.42 | NA | NA | Isoform Beta-2 of DNA | TOP2B |
| | | | | topoisomerase 2-beta [749] | |
| DMCLEK(ac)DTLGLFLR | −0.41 | NA | NA | Isoform 1 of U5 small nuclear | SNRNP200 |
| (SEQ ID NO: 530) | | | | ribonucleoprotein 200 kDa | |
| | | | | helicase [745] | |
| IAAYK(ac)SILQER | −0.41 | NA | 0.00 | X-ray repair cross-complementing | XRCC5 |
| (SEQ ID NO: 531) | | | | protein 5 [265] | |
| YAVLYQPLFDK(ac)R | −0.41 | NA | 0.00 | Nucleosome assembly protein 1- | NAP1L1 |
| (SEQ ID NO: 532) | | | | like 1 [116] | |
| SHLAK(ac)EGLYQYK | −0.41 | 0.00 | 0.00 | Eukaryotic translation initiation | EIF3A |
| (SEQ ID NO: 533) | | | | factor 3 subunit A [68] | |
| YKDAIHFYNK(ac)SLAEHR | −0.40 | NA | 0.00 | Stress-induced-phosphoprotein | STIP1 |
| (SEQ ID NO: 534) | | | | 1 [325] | |
| YIGK(ac)TMDYR (SEQ ID NO: 535) | −0.39 | NA | NA | 39S ribosomal protein L24 | MRPL24 |
| | | | | mitochondrial [100] | |
| GLCEK(ac)PLASAAAK | −0.39 | NA | 0.00 | Isoform 2 of Transportin-3 [513] | TNPO3 |
| (SEQ ID NO: 536) | | | | | |
| GK(ac)FGNVYLAR | −0.39 | NA | NA | Isoform 1 of Aurora kinase C | AURKC |
| (SEQ ID NO: 537) | | | | [53] | |
| LWSK(ac)AIFAGYK | 0.38 | 0.00 | 0.00 | 60S ribosomal protein L35a [8] | RPL35A |
| (SEQ ID NO: 538) | | | | | |
| LQVLDPVPK(ac)PVIK | 0.39 | NA | NA | CD48 antigen [133] | CD48 |
| (SEQ ID NO: 539) | | | | | |
| ELK(ac)EIQYGIR | 0.39 | 0.00 | 0.00 | Branched-chain-amino-acid | BCAT2 |
| (SEQ ID NO: 540) | | | | aminotransferase [337] | |
| GYPTIK(ac)FFR (SEQ ID NO: 541) | 0.39 | 0.00 | 0.00 | Protein disulfide-isomerase [103] | P4HB |
| IHGVGFK(ac)K (SEQ ID NO: 542) | 0.39 | NA | 0.00 | 60S ribosomal protein L31 [39] | RPL31 |
| ISGASEK(ac)DIVHSGLAYTMER | 0.39 | 0.00 | 0.00 | Glutamate dehydrogenase 1 | GLUD1 |
| (SEQ ID NO: 543) | | | | mitochondrial [503] | |
| PK(ac)PLLFK (SEQ ID NO: 544) | 0.39 | NA | NA | Isoform 1 of UDP- | UGGT1 |
| | | | | glucose:glycoprotein | |
| | | | | glucosyltransferase 1 [176] | |
| FNNFLK(ac)ALQEK | 0.39 | NA | NA | X-ray repair cross-complementing | XRCC5 |
| (SEQ ID NO: 545) | | | | protein 5 [660] | |
| AVDLIQK(ac)HK (SEQ ID NO: 546) | 0.39 | 0.00 | 0.00 | Flap endonuclease 1 [252] | FEN1 |
| AEEILEK(ac)GLK | 0.39 | 0.00 | NA | Isoform 1 of 60S ribosomal | RPL11 |
| (SEQ ID NO: 547) | | | | protein L11 [85] | |
| LFDYFPK(ac)PYPNSEAAR | 0.39 | NA | NA | Cytochrome c1 heme protein | CYC1 |
| (SEQ ID NO: 548) | | | | mitochondrial [177] | |
| AHLQK(ac)LMSDK | 0.39 | NA | 0.00 | Glycyl-tRNA synthetase [224] | GARS |
| (SEQ ID NO: 549) | | | | | |
| TFK(ac)GVVDVVMK | 0.39 | 0.00 | 0.00 | Elongation factor G 2 | GFM2 |
| (SEQ ID NO: 550) | | | | mitochondrial precursor [265] | |
| AAWEHMK(ac)K (SEQ ID NO: 551) | 0.39 | NA | NA | Peroxisomal multifunctional | HSD17B4 |
| | | | | enzyme type 2 [139] | |
| STVAQLVK(ac)R (SEQ ID NO: 552) | 0.39 | 0.00 | NA | ATP synthase subunit alpha | ATP5A1 |
| | | | | mitochondrial [261] | |
| YNIEK(ac)DIAAHIK | 0.40 | 0.00 | 0.00 | Dynein light chain 1 cytoplasmic | DYNLL1 |
| (SEQ ID NO: 553) | | | | [36] | |
| HFAFK(ac)MASGAANVVGPK | 0.40 | NA | 0.00 | Protein FAM3C [72] | FAM3C |
| (SEQ ID NO: 554) | | | | | |
| TDFDKNK(ac)IWYEHR | 0.40 | NA | 0.00 | Isocitrate dehydrogenase [NADP] | IDH2 |
| (SEQ ID NO: 555) | | | | mitochondrial [282] | |
| HGYIK(ac)GIVK (SEQ ID NO: 556) | 0.40 | NA | NA | 60S ribosomal protein L8 [42] | RPL8 |
| FAK(ac)PVYPGQTLQTEMWK | 0.40 | NA | NA | Peroxisomal multifunctional enzyme | HSD17B4 |
| (SEQ ID NO: 557) | | | | type 2 [565] | |
| NCSSFLIK(ac)R (SEQ ID NO: 558) | 0.40 | 0.00 | 0.00 | 60S ribosomal protein L28 [19] | RPL28 |
| NK(ac)IAGYVTHLMK | 0.40 | 0.00 | 0.00 | 40S ribosomal protein S17 [49] | RPS17, |
| (SEQ ID NO: 559) | | | | | RPS17L |
| AVTK(ac)AQK(ac)K | 0.40 | 0.00 | NA | Histone H2B type 2-E [21:24] | HIST2H2BE |
| (SEQ ID NO: 560) | | | | | |
| VK(ac)ILFNK (SEQ ID NO: 561) | 0.40 | NA | NA | Isoform 1 of Polypyrimidine tract- | PTBP1 |
| | | | | binding protein 1 [368] | |
| VK(ac)FIHDQTSPNPK | 0.40 | 0.00 | 0.00 | Tricarboxylate transport protein | SLC25A1 |
| (SEQ ID NO: 562) | | | | mitochondrial [149] | |
| YFDEK(ac)IAK (SEQ ID NO: 563) | 0.41 | 0.00 | 0.00 | D-beta-hydroxybutyrate | BDH1 |
| | | | | dehydrogenase mitochondrial [280] | |
| LSLLSK(ac)FR (SEQ ID NO: 564) | 0.41 | 0.00 | 0.00 | Mitochondrial trifunctional | HADHB |
| | | | | protein beta subunit | |
| | | | | (Fragment) [100] | |
| VK(ac)NELFK (SEQ ID NO: 565) | 0.41 | 0.00 | NA | Isoform 1 of Hydroxyacyl-coenzyme | HADH |
| | | | | A dehydrogenase mitochondrial | |
| | | | | [127] | |
| EVGKDVSDEK(ac)LR | 0.41 | NA | NA | Citrate synthase mitochondrial | CS |
| (SEQ ID NO: 566) | | | | [327] | |
| SFLLK(ac)DSETSQR | 0.41 | 0.00 | 0.00 | Serine hydroxymethyltransferase | SHMT2 |
| (SEQ ID NO: 567) | | | | mitochondrial [474] | |
| VFEK(ac)YGR (SEQ ID NO: 568) | 0.41 | NA | NA | Serine/arginine-rich splicing | SRSF2 |
| | | | | factor 2 [36] | |
| NSNPALNDNLEK(ac)GLLK | 0.41 | NA | 0.00 | Chloride intracellular channel | CLIC1 |
| (SEQ ID NO: 569) | | | | protein 1 [131] | |
| LAAFGQLHK(ac)VLGMDPLPSK | 0.41 | NA | NA | Isoform 5 of Interleukin enhancer- | ILF3 |
| (SEQ ID NO: 570) | | | | binding factor 3 [332] | |
| DVSYQGGK(ac)SIK | 0.42 | NA | 0.00 | Isoform 1 of Histone-lysine N- | MLL3 |
| (SEQ ID NO: 571) | | | | methyltransferase MLL3 [758] | |
| SIDLK(ac)DK (SEQ ID NO: 572) | 0.42 | NA | NA | 60 kDa heat shock protein | HSPD1 |
| | | | | mitochondrial [87] | |
| MTDK(ac)CFR (SEQ ID NO: 573) | 0.42 | NA | 0.00 | Mitochondrial import inner | TIMM13 |
| | | | | membrane translocase subunit Tim13 | |
| | | | | [45] | |
| HAHGDQYK(ac)ATDFVADR | 0.42 | 0.00 | 0.00 | Isocitrate dehydrogenase [NADP] | IDH2 |
| (SEQ ID NO: 574) | | | | mitochondrial [180] | |
| (ac)ACGLVASNLNLK(ac)PGECLR | 0.42 | 0.00 | 0.00 | Galectin-1 [13] | LGALS1 |
| (SEQ ID NO: 575) | | | | | |
| HVVFGK(ac)VK (SEQ ID NO: 576) | 0.42 | NA | 0.00 | Peptidylprolyl isomerase A | PPIAP26 |
| | | | | (Cyclophilin A) (PPIA) pseudogene | |
| | | | | [141] | |
| AVLIDK(ac)DQSPK | 0.43 | NA | 0.00 | Isoform 1 of 3-hydroxyisobutyryl- | HIBCH |
| (SEQ ID NO: 577) | | | | CoA hydrolase mitochondrial [353] | |
| YDDPEVQK(ac)DIK | 0.43 | 0.00 | 0.00 | Stress-70 protein mitochondrial | HSPA9 |
| (SEQ ID NO: 578) | | | | [135] | |
| DDNGK(ac)PYVLPSVR | 0.43 | 0.00 | 0.00 | Aspartate aminotransferase | GOT2 |
| (SEQ ID NO: 579) | | | | mitochondrial [73] | |
| AGEATVK(ac)FLK | 0.43 | NA | NA | 39S ribosomal protein L9 | MRPL9 |
| (SEQ ID NO: 580) | | | | mitochondrial [165] | |
| RNPDTQWITK(ac)PVHK | 0.43 | 0.00 | NA | 60S ribosomal protein L15 [153] | RPL15 |
| (SEQ ID NO: 581) | | | | | |
| LQDFK(ac)SFLLK | 0.43 | 0.00 | 0.00 | Serine hydroxymethyltransferase | SHMT2 |
| (SEQ ID NO: 582) | | | | mitochondrial [469] | |
| EFALK(ac)HLPNDPMFK | 0.43 | 0.00 | NA | Citrate synthase mitochondrial | CS |
| (SEQ ID NO: 583) | | | | [366] | |
| TACAINK(ac)VLMGR | 0.43 | NA | 0.00 | Isoform 1 of Protein FAM98A [297] | FAM98A |
| (SEQ ID NO: 584) | | | | | |
| LMK(ac)YLLEQLK | 0.43 | 0.00 | NA | Isoform 1 of Protein MB21D1 [414] | MB21D1 |
| (SEQ ID NO: 585) | | | | | |
| ASEIGEK(ac)YR (SEQ ID NO: 586) | 0.44 | 0.00 | 0.00 | Conserved hypothetical protein | KIAA0947 |
| | | | | [484] | |
| ITK(ac)SDGIR (SEQ ID NO: 587) | 0.44 | 0.00 | 0.00 | ADP/ATP translocase 3 [166] | SLC25A6 |
| ELEAENYHDIK(ac)R | 0.44 | NA | NA | Isoform Long of Spectrin beta | SPTBN1 |
| (SEQ ID NO: 588) | | | | chain brain 1 [497] | |
| NLHSK(ac)WLK (SEQ ID NO: 589) | 0.44 | NA | NA | Isoform Long of Spectrin beta | SPTBN1 |
| | | | | chain brain 1 [1312] | |
| LTSLNVK(ac)YNNDK | 0.44 | 0.00 | 0.00 | regulator of differentiation 1 | ROD1 |
| (SEQ ID NO: 590) | | | | isoform 2 [236] | |
| VASK(ac)QLEEEDGSR | 0.44 | NA | NA | Coatomer subunit gamma [238] | COPG |
| (SEQ ID NO: 591) | | | | | |
| TAWLDGK(ac)HVVFGK | 0.44 | 0.00 | 0.00 | Peptidyl-prolyl cis-trans | PPIB |
| (SEQ ID NO: 592) | | | | isomerase B [165] | |
| VWNYK(ac)LR (SEQ ID NO: 593) | 0.45 | NA | 0.00 | Isoform 1 of Coatomer subunit | COPA |
| | | | | alpha [81] | |
| K(ac)NINCSIEESFQR | 0.45 | 0.00 | 0.00 | Hydroxymethylglutaryl-CoA lyase | HMGCL |
| (SEQ ID NO: 594) | | | | mitochondrial [137] | |
| YEK(ac)DIAAYR (SEQ ID NO: 595) | 0.45 | NA | 0.00 | High mobility group protein B2 | HMGB2 |
| | | | | [157] | |
| FYK(ac)DVLEVGELAK | 0.45 | 0.00 | 0.00 | Isoform 1 of N-alpha- | NAA50 |
| (SEQ ID NO: 596) | | | | acetyltransferase 50 NatE | |
| | | | | catalytic subunit [37] | |
| VVK(ac)QASEGPLK | 0.45 | 0.00 | 0.00 | Glyceraldehyde-3-phosphate | GAPDH |
| (SEQ ID NO: 597) | | | | dehydrogenase [263] | |
| TSSAFVGK(ac)TPEASPEPK | 0.46 | NA | NA | Isoform 1 of 26S proteasome non- | PSMD1 |
| (SEQ ID NO: 598) | | | | ATPase regulatory subunit 1 [310] | |
| ADGIVSK(ac)NF (SEQ ID NO: 599) | 0.46 | NA | NA | 40S ribosomal protein S21 [81] | RPS21 |
| ISEQSDAK(ac)LK | 0.46 | 0.00 | 0.00 | ATP synthase subunit alpha | ATP5A1 |
| (SEQ ID NO: 600) | | | | mitochondrial [539] | |
| K(ac)SSETLR (SEQ ID NO: 601) | 0.46 | 0.00 | NA | Isoform CNPI of 23-cyclic- | CNP |
| | | | | nucleotide 3- | |
| | | | | phosphodiesterase [177] | |
| SGLLDK(ac)WK (SEQ ID NO: 602) | 0.46 | 0.00 | 0.00 | Signal peptidase complex subunit | LOC653566, |
| | | | | 2 [38] | SPCS2 |
| LNLDK(ac)MMEQK | 0.46 | 0.00 | NA | Dihydrolipoyl dehydrogenase | DLD |
| (SEQ ID NO: 603) | | | | mitochondrial [122] | |
| LAK(ac)YFFNK (SEQ ID NO: 604) | 0.46 | NA | 0.00 | Periodic tryptophan protein 2 | PWP2 |
| | | | | homolog [279] | |
| NSFDCFK(ac)K (SEQ ID NO: 605) | 0.46 | NA | 0.00 | Calcium-binding mitochondrial | SLC25A13 |
| | | | | carrier protein Aralar2 [379] | |
| AGTFK(ac)MVFTPK | 0.46 | 0.00 | 0.00 | Isocitrate dehydrogenase [NADP] | IDH2 |
| (SEQ ID NO: 606) | | | | mitochondrial [193] | |
| SLNLK(ac)HIK (SEQ ID NO: 607) | 0.46 | 0.00 | 0.00 | HLA-B associated transcript | SNORD84, |
| | | | | 1 [188] | DDX39B |
| DHCVAHK(ac)LFNNLK | 0.46 | 0.00 | 0.00 | Cytochrome b-c1 complex subunit 6 | UQCRH |
| (SEQ ID NO: 608) | | | | mitochondrial [85] | |
| NVK(ac)FVLK (SEQ ID NO: 609) | 0.47 | NA | NA | Translocon-associated protein | SSR3 |
| | | | | subunit gamma [81] | |
| VLTVINQTQK(ac)ENLR | 0.47 | NA | NA | 60S ribosomal protein L35 [66] | RPL35 |
| (SEQ ID NO: 610) | | | | | |
| GAK(ac)PVVVLQK | 0.47 | NA | 0.00 | Isoform 2 of Nipped-B-like protein | NIPBL |
| (SEQ ID NO: 611) | | | | [1000] | |
| ELEK(ac)VCNPIITK | 0.47 | 0.00 | 0.00 | Isoform 1 of Heat shock cognate | HSPA8 |
| (SEQ ID NO: 612) | | | | 71 kDa protein [601] | |
| IHDVLCK(ac)LVEK | 0.47 | NA | NA | Leucine-rich PPR motif-containing | LRPPRC |
| (SEQ ID NO: 613) | | | | protein mitochondrial [864] | |
| K(ac)FAYLGR (SEQ ID NO: 614) | 0.47 | 0.00 | 0.00 | 60S ribosomal protein L13a [134] | SNORD32A, |
| | | | | | RPL13A, |
| | | | | | SNORD33, |
| | | | | | SNORD34 |
| SK(ac)GK(ac)NSDEEAPK | 0.47 | NA | NA | Isoform 2 of Inhibitor of growth | ING4 |
| (SEQ ID NO: 615) | | | | protein 4 [145:147] | |
| K(ac)EYVFADSK (SEQ ID NO: 616) | 0.47 | NA | NA | Isoform 1 of Phosphati- | INPP5D, |
| | | | | dylinositol-345-trisphosphate | LOC646743 |
| | | | | 5-phosphatase 1 [371] | |
| EIAENALGK(ac)HK | 0.47 | NA | NA | Isoform 1 of Heterogeneous nuclear | HNRNPH3 |
| (SEQ ID NO: 617) | | | | ribonucleoprotein H3 [76] | |
| FQK(ac)ELER (SEQ ID NO: 618) | 0.48 | NA | NA | 39S ribosomal protein L44 | MRPL44 |
| | | | | mitochondrial [43] | |
| GFSLLATEDK(ac)EALKK | 0.48 | NA | NA | Poly [ADP-ribose] polymerase | PARP1 |
| (SEQ ID NO: 619) | | | | 1 [192] | |
| VIISAPSADAPMFVMGVNHEK(ac)YDNSLK | 0.48 | NA | 0.00 | Glyceraldehyde-3-phosphate | GAPDH |
| (SEQ ID NO: 620) | | | | dehydrogenase [139] | |
| GAGTDEK(ac)TLTR | 0.48 | 0.00 | 0.00 | Annexin A6 [620] | ANXA6 |
| (SEQ ID NO: 621) | | | | | |
| GPLDK(ac)WR (SEQ ID NO: 622) | 0.48 | 0.00 | 0.00 | 60S ribosomal protein L10- | RPL10L |
| | | | | like [208] | |
| EFHQAGK(ac)PIGLCCIAPVLAAK | 0.48 | NA | NA | Isoform Long of ES1 protein | C21orf33 |
| (SEQ ID NO: 623) | | | | homolog mitochondrial [171] | |
| TTLLYK(ac)LK (SEQ ID NO: 624) | 0.49 | 0.00 | 0.00 | ADP-ribosylation factor-like | ARL11 |
| | | | | protein 11 [31] | |
| VPNDK(ac)YYGAQTVR | 0.49 | 0.00 | 0.00 | Isoform Mitochondrial of Fumarate | FH |
| (SEQ ID NO: 625) | | | | hydratase mitochondrial [66] | |
| ADTLTPEECQQFK(ac)K | 0.49 | NA | 0.00 | Isoform 2 of Septin-7 [207] | 7-Sep |
| (SEQ ID NO: 626) | | | | | |
| AFAK(ac)ELFLGK | 0.49 | 0.00 | 0.00 | Acyl-CoA dehydrogenase family | ACAD9 |
| (SEQ ID NO: 627) | | | | member 9 mitochondrial [41] | |
| SYVSEK(ac)DVTSAK | 0.49 | 0.00 | 0.00 | Leucine-rich PPR motif-containing | LRPPRC |
| (SEQ ID NO: 628) | | | | protein mitochondrial [1332] | |
| LIGK(ac)QGR (SEQ ID NO: 629) | 0.49 | NA | NA | cDNA FLJ56047 highly similar to | AKAP1 |
| | | | | A kinase anchor protein 1 | |
| | | | | mitochondrial [667] | |
| EHTALLK(ac)IEGVYAR | 0.49 | 0.00 | 0.00 | 60S ribosomal protein L35a [29] | RPL35A |
| (SEQ ID NO: 630) | | | | | |
| FNDGSDEK(ac)KK | 0.49 | NA | NA | ATP synthase subunit alpha | ATP5A1 |
| (SEQ ID NO: 631) | | | | mitochondrial [239] | |
| FSK(ac)SQLDIIIHSLK | 0.50 | NA | NA | X-ray repair cross-complementing | XRCC5 |
| (SEQ ID NO: 632) | | | | protein 5 [144] | |
| LTCEEEEEK(ac)IFGR | 0.51 | NA | NA | Isoform Short of Probable global | SMARCA2 |
| (SEQ ID NO: 633) | | | | transcription activator SNF2L2 | |
| | | | | [1302] | |
| NDEELNK(ac)LLGR | 0.52 | 0.00 | 0.00 | Histone H2A type 1-B/E [96] | HIST1H2AE, |
| (SEQ ID NO: 634) | | | | | HIST1H2AB, |
| | | | | | HIST1H2AD, |
| | | | | | HIST1H2AG, |
| | | | | | HIST1H2AK, |
| | | | | | HIST1H2AL, |
| | | | | | HIST1H2AM, |
| | | | | | HIST1H2AJ, |
| | | | | | HIST1H2AI |
| ALEAVFGK(ac)YGR | 0.52 | NA | NA | Heterogeneous nuclear | RBMX |
| (SEQ ID NO: 635) | | | | ribonucleoprotein G [30] | |
| TFK(ac)TVFAEHISDECK | 0.52 | 0.00 | 0.00 | 60S ribosomal protein L3 [103] | SNORD43, |
| (SEQ ID NO: 636) | | | | | RPL3 |
| YK(ac)PESEELTAER | 0.52 | 0.00 | 0.00 | Protein disulfide-isomerase [328] | P4HB |
| (SEQ ID NO: 637) | | | | | |
| NAEK(ac)YAEEDRR | 0.52 | 0.00 | 0.00 | Stress-70 protein mitochondrial | HSPA9 |
| (SEQ ID NO: 638) | | | | [567] | |
| SKSDPIMLLK(ac)DR | 0.53 | 0.00 | 0.00 | pyruvate dehydrogenase E1 | PDHA1 |
| (SEQ ID NO: 639) | | | | component subunit alpha somatic | |
| | | | | form mitochondrial isoform 2 | |
| | | | | precursor [359] | |
| GVDPK(ac)FLR (SEQ ID NO: 640) | 0.53 | NA | NA | Ribosomal protein L29 [38] | RPL29, |
| | | | | | RPL29P4 |
| LIK(ac)DGLIIR (SEQ ID NO: 641) | 0.53 | NA | NA | 60S ribosomal protein L19 [46] | RPL19 |
| HLLPK(ac)IR (SEQ ID NO: 642) | 0.53 | NA | NA | 6-phosphogluconate dehydrogenase | PGD |
| | | | | decarboxylating [253] | |
| LGK(ac)PSLVR (SEQ ID NO: 643) | 0.53 | NA | NA | TOB3 [280] | ATAD3B |
| EQIYK(ac)LAK (SEQ ID NO: 644) | 0.54 | 0.00 | NA | 40S ribosomal protein S13 [39] | RPS13 |
| VEYFLK(ac)WK (SEQ ID NO: 645) | 0.54 | 0.00 | 0.00 | Chromobox protein homolog 3 [50] | CBX3 |
| HYK(ac)TDFDK (SEQ ID NO: 646) | 0.54 | 0.00 | 0.00 | Isocitrate dehydrogenase [NADP] | IDH2 |
| | | | | mitochondrial [275] | |
| EGYAWAEDK(ac)EHCEEYGR | 0.54 | NA | 0.00 | tRNA-splicing ligase RtcB homolog | C22orf28 |
| (SEQ ID NO: 647) | | | | [190] | |
| LAYIAHPK(ac)LGK | 0.55 | NA | NA | Ribosomal protein L29 [103] | RPL29, |
| (SEQ ID NO: 648) | | | | | RPL29P4 |
| IFSQETLTK(ac)AQILK | 0.55 | 0.00 | NA | Prenylcysteine oxidase 1 [415] | PCYOX1 |
| (SEQ ID NO: 649) | | | | | |
| ALTGGIAHLFK(ac)QNK | 0.55 | 0.00 | 0.00 | Dihydrolipoyl dehydrogenase | DLD |
| (SEQ ID NO: 650) | | | | mitochondrial [143] | |
| FYVK(ac)DHR (SEQ ID NO: 651) | 0.55 | NA | 0.00 | CDGSH iron-sulfur domain- | CISD1 |
| | | | | containing protein 1 [37] | |
| SPVNTK(ac)SLFK | 0.55 | NA | NA | Isoform 1 of DNA-dependent protein | PRKDC |
| (SEQ ID NO: 652) | | | | kinase catalytic subunit [1057] | |
| HIVK(ac)EFK (SEQ ID NO: 653) | 0.56 | 0.00 | 0.00 | Stress-70 protein mitochondrial | HSPA9 |
| | | | | [288] | |
| MYKEEGLK(ac)AFYK | 0.56 | 0.00 | 0.00 | Isoform A of Phosphate carrier | SLC25A3 |
| (SEQ ID NO: 654) | | | | protein mitochondrial [214] | |
| LDAQVK(ac)ELVLK | 0.56 | 0.00 | NA | Dolichyl-diphospho- | RPN1 |
| (SEQ ID NO: 655) | | | | oligosaccharide-- | |
| | | | | protein glycosyltransferase | |
| | | | | subunit 1 precursor [598] | |
| GYPTLK(ac)LFKPGQEAVK | 0.56 | NA | NA | Thioredoxin domain-containing | TXNDC5, |
| (SEQ ID NO: 656) | | | | protein 5 [140] | MUTED, |
| | | | | | MUTED- |
| | | | | | TXNDC5 |
| EVCFACVDGK(ac)EFR | 0.57 | NA | 0.00 | Isoform 1 of Clathrin heavy | CLTC |
| (SEQ ID NO: 657) | | | | chain 1 [1264] | |
| YK(ac)EALEQCR (SEQ ID NO: 658) | 0.57 | 0.00 | NA | Isoform 2 of Antigen peptide | TAP2 |
| | | | | transporter 2 [356] | |
| YELTGK(ac)FER (SEQ ID NO: 659) | 0.57 | 0.00 | 0.00 | Annexin A6 [81] | ANXA6 |
| FIK(ac)IDGK (SEQ ID NO: 660) | 0.57 | NA | NA | 40S ribosomal protein S4 X | RPS4X |
| | | | | isoform [71] | |
| LAMVK(ac)AEPDVK | 0.58 | NA | 0.00 | NADH dehydrogenase [ubiquinone] | NDUFA5 |
| (SEQ ID NO: 661) | | | | 1 alpha subcomplex subunit | |
| | | | | 5 [60] | |
| NEK(ac)LFYR (SEQ ID NO: 662) | 0.58 | NA | 0.00 | NADP-dependent malic enzyme | ME3 |
| | | | | mitochondrial [119] | |
| K(ac)FVCPECSK (SEQ ID NO: 663) | 0.58 | NA | NA | Isoform 1 of Transcription factor | SP3 |
| | | | | Sp3 [680] | |
| DLSLDDFK(ac)GK | 0.58 | 0.00 | 0.00 | Thioredoxin-dependent peroxide | PRDX3 |
| (SEQ ID NO: 664) | | | | reductase mitochondrial [91] | |
| DLTDYLMK(ac)ILTER | 0.58 | NA | NA | Beta-actin-like protein 2 [192] | ACTBL2 |
| (SEQ ID NO: 665) | | | | | |
| HGASPELQK(ac)QIR | 0.58 | NA | NA | Peroxisomal membrane protein 11B | PEX11B |
| (SEQ ID NO: 666) | | | | [43] | |
| LNQLK(ac)PGLQYK | 0.59 | NA | NA | Isoform 5 of Interleukin enhancer- | ILF3 |
| (SEQ ID NO: 667) | | | | binding factor 3 [413] | |
| ISK(ac)LYGDLK (SEQ ID NO: 668) | 0.59 | 0.00 | NA | cDNA FLJ61478 highly similar to | SDHA |
| | | | | Succinate dehydrogenase | |
| | | | | (ubiquinone) flavoprotein subunit | |
| | | | | mitochondrial [541] | |
| LTSDDVK(ac)EQIYK | 0.60 | 0.00 | 0.00 | 40S ribosomal protein S13 [34] | RPS13 |
| (SEQ ID NO: 669) | | | | | |
| SCNCLLLK(ac)VNQIGSVTESLQACK | 0.60 | NA | 0.00 | Isoform alpha-enolase of Alpha- | ENO1 |
| (SEQ ID NO: 670) | | | | enolase [343] | |
| VTCIDPNPNFEK(ac)FLIK | 0.60 | 0.00 | 0.00 | Methyltransferase-like protein 7A | METTL7A |
| (SEQ ID NO: 671) | | | | [105] | |
| LILGLMMPPAHYDAK(ac)QLK | 0.60 | 0.00 | 0.00 | Annexin A6 [442] | ANXA6 |
| (SEQ ID NO: 672) | | | | | |
| EVK(ac)LLLLGAGESGK | 0.60 | 0.00 | NA | Guanine nucleotide-binding | GNAI3 |
| (SEQ ID NO: 673) | | | | protein G(k) subunit alpha [35] | |
| EAKPDELMDSK(ac)LR | 0.60 | 0.00 | NA | Isoform 1 of Vesicle-associated | VAPA |
| (SEQ ID NO: 674) | | | | membrane protein-associated | |
| | | | | protein A [125] | |
| YNWSAK(ac)AK (SEQ ID NO: 675) | 0.60 | NA | NA | 60S ribosomal protein L37 [52] | RPL37 |
| GNK(ac)PWISLPR | 0.60 | NA | NA | 40S ribosomal protein S4 X | RPS4X |
| (SEQ ID NO: 676) | | | | isoform [233] | |
| EVVEAHVDQK(ac)NK | 0.61 | NA | NA | Isoform Long of ES1 protein | C21orf33 |
| (SEQ ID NO: 677) | | | | homolog mitochondrial [233] | |
| LENDK(ac)SFR (SEQ ID NO: 678) | 0.61 | 0.00 | 0.00 | Isoform 1 of Enoyl-CoA delta | ECI1 |
| | | | | isomerase 1 mitochondrial [89] | |
| LVAMK(ac)FLR (SEQ ID NO: 679) | 0.61 | 0.00 | 0.00 | Isoform 1 of Nucleoside | NME1, NME2, |
| | | | | diphosphate kinase B [39] | NME1-NME2 |
| KVPFGK(ac)TYCCDLK | 0.62 | NA | NA | Mitochondrial carrier homolog | MTCH2 |
| (SEQ ID NO: 680) | | | | 2 [293] | |
| TK(ac)FVDGVSTVAR | 0.62 | NA | NA | Isoform 1 of Structural | SMC2 |
| (SEQ ID NO: 681) | | | | maintenance of chromosomes | |
| | | | | protein 2 [1160] | |
| LAQLEEAK(ac)QASIQHIQNAIDTEK | 0.63 | NA | NA | ATP synthase subunit b | ATP5F1 |
| (SEQ ID NO: 682) | | | | mitochondrial [139] | |
| IPVPVQK(ac)NIDQQIK | 0.64 | NA | NA | Isoform 1 of Pre-mRNA-splicing | PRPF38B |
| (SEQ ID NO: 683) | | | | factor 38B [227] | |
| YQLDKDGVVLFK(ac)K | 0.64 | NA | NA | Protein disulfide-isomerase [207] | P4HB |
| (SEQ ID NO: 684) | | | | | |
| VDIRPQLLK(ac)NALQR | 0.64 | NA | NA | Isoform 1 of Heterochromatin | HP1BP3 |
| (SEQ ID NO: 685) | | | | protein 1-binding protein 3 [302] | |
| NQK(ac)LTATTQK | 0.64 | NA | NA | Nucleoprotein TPR [748] | TPR |
| (SEQ ID NO: 686) | | | | | |
| YYK(ac)NIGLGFK | 0.65 | NA | NA | 40S ribosomal protein S11 [38] | RPS11 |
| (SEQ ID NO: 687) | | | | | |
| SGK(ac)YDLDFK (SEQ ID NO: 688) | 0.65 | 0.00 | 0.00 | Isoform alpha-enolase of Alpha- | ENO1 |
| | | | | enolase [256] | |
| LYGDLK(ac)HLK (SEQ ID NO: 689) | 0.65 | 0.00 | 0.00 | cDNA FLJ61478 highly similar to | SDHA |
| | | | | Succinate dehydrogenase | |
| | | | | (ubiquinone) flavoprotein subunit | |
| | | | | mitochondrial [547] | |
| VENEAEK(ac)DLQCHAPVR | 0.65 | NA | NA | Isoform 1 of Bromodomain- | BRD7 |
| (SEQ ID NO: 690) | | | | containing protein 7 [103] | |
| DSLYK(ac)DAMQYASESK | 0.65 | NA | NA | Isoform 1 of Clathrin heavy | CLTC |
| (SEQ ID NO: 691) | | | | chain 1 [1535] | |
| TISAK(ac)YAEER | 0.65 | NA | 0.00 | Isoform 1 of Myosin-9 [1459] | MYH9 |
| (SEQ ID NO: 692) | | | | | |
| (ac)ASNK(ac)TTLQK | 0.66 | 0.00 | 0.00 | Chromobox protein homolog 3 [5] | CBX3 |
| (SEQ ID NO: 693) | | | | | |
| GYLADPAK(ac)FPEAR | 0.66 | NA | NA | 39S ribosomal protein L15 | MRPL15 |
| (SEQ ID NO: 694) | | | | mitochondrial [228] | |
| SCEIK(ac)FLTFK | 0.66 | 0.00 | NA | Isoform 2 of 39S ribosomal | MRPL39 |
| (SEQ ID NO: 695) | | | | protein L39 mitochondrial | |
| | | | | [126] | |
| SELK(ac)HAK (SEQ ID NO: 696) | 0.66 | NA | NA | Isoform Long of Delta-1-pyrroline- | ALDH18A1 |
| | | | | 5-carboxylate synthase [68] | |
| SCGLNPK(ac)LEK | 0.66 | NA | NA | Isoform 5 of Transcriptional | ATRX |
| (SEQ ID NO: 697) | | | | regulator ATRX [468] | |
| EVK(ac)VLLDQLR | 0.67 | NA | NA | Isoform 1 of Adipocyte plasma | C20orf3 |
| (SEQ ID NO: 698) | | | | membrane-associated protein [250] | |
| KAVTK(ac)VQK (SEQ ID NO: 699) | 0.67 | 0.00 | 0.00 | Histone H2B type 2-F [21] | HIST2H2BF |
| ILQK(ac)QGFK (SEQ ID NO: 700) | 0.67 | 0.00 | 0.00 | Dihydrolipoyl dehydrogenase | DLD |
| | | | | mitochondrial [267] | |
| NK(ac)FGAPQK (SEQ ID NO: 701) | 0.68 | NA | 0.00 | Trifunctional enzyme subunit alpha | HADHA |
| | | | | mitochondrial [353] | |
| VTAVHK(ac)ANIMK | 0.68 | NA | 0.00 | Isocitrate dehydrogenase [NAD] | IDH3G |
| (SEQ ID NO: 702) | | | | subunit gamma mitochondrial [221] | |
| ACPEK(ac)HFAFK | 0.68 | 0.00 | 0.00 | Protein FAM3C [67] | FAM3C |
| (SEQ ID NO: 703) | | | | | |
| KPTLDK(ac)PSPETFVK | 0.68 | 0.00 | NA | Estradiol 17-beta-dehydrogenase | HSD17B12 |
| (SEQ ID NO: 704) | | | | 12 [249] | |
| QLAQK(ac)YNCDK | 0.68 | 0.00 | 0.00 | Ubiquitin-60S ribosomal protein | UBA52 |
| (SEQ ID NO: 705) | | | | L40 [88] | |
| YTAQVDAEEK(ac)EDVK | 0.68 | 0.00 | 0.00 | Isoform 1 of ATP synthase subunit | ATP5H |
| (SEQ ID NO: 706) | | | | d mitochondrial [95] | |
| PLISPQTSHK(ac)TLSK | 0.69 | NA | NA | Isoform 2 of PDZ domain-containing | PDZD2 |
| (SEQ ID NO: 707) | | | | protein 2 [1726] | |
| ADK(ac)LAEEHSS | 0.70 | 0.00 | 0.00 | ATP synthase subunit beta | ATP5B |
| (SEQ ID NO: 708) | | | | mitochondrial [522] | |
| DFGSFDK(ac)FK (SEQ ID NO: 709) | 0.70 | NA | 0.00 | Superoxide dismutase [Mn] | SOD2 |
| | | | | mitochondrial [130] | |
| KIDK(ac)YTEVLK | 0.70 | 0.00 | NA | 60S ribosomal protein L13a [191] | SNORD32A, |
| (SEQ ID NO: 710) | | | | | RPL13A, |
| | | | | | SNORD33, |
| | | | | | SNORD34 |
| CSK(ac)LPSSTK(ac)SGWPR | 0.70 | 0.00 | 0.00 | cDNA FLJ75110 highly similar to | PHF15 |
| (SEQ ID NO: 711) | | | | Homo sapiens PHD finger protein | |
| | | | | 15 (PHF15) mRNA 3[2:38] | |
| KAINK(ac)AQK (SEQ ID NO: 712) | 0.70 | 0.00 | 0.00 | Histone H2B type 1-M [21] | HIST1H2BM |
| EFPEK(ac)QELHR | 0.70 | NA | NA | Protein zer-1 homolog [584] | ZER1 |
| (SEQ ID NO: 713) | | | | | |
| TDLEK(ac)DIISDTSGDFR | 0.71 | 0.00 | 0.00 | Putative annexin A2-like | ANXA2P2 |
| (SEQ ID NO: 714) | | | | protein [157] | |
| TPAQYDASELK(ac)ASMK | 0.72 | 0.00 | NA | Putative annexin A2-like | ANXA2P2 |
| (SEQ ID NO: 715) | | | | protein [115] | |
| LTCYLCK(ac)QK (SEQ ID NO: 716) | 0.72 | NA | NA | Isoform 1 of Bromodomain- | BRD1 |
| | | | | containing protein 1 [33]1 | |
| AINK(ac)AQK(ac)K | 0.72 | NA | NA | Histone H2B type 1-M [21:24] | HIST1H2BM |
| (SEQ ID NO: 717) | | | | | |
| KGIEK(ac)NLGIGK | 0.72 | 0.00 | 0.00 | Malate dehydrogenase mitochondrial | MDH2 |
| (SEQ ID NO: 718) | | | | [301] | |
| DNGK(ac)HALIIYDDLSK | 0.72 | 0.00 | NA | ATP synthase subunit alpha | ATP5A1 |
| (SEQ ID NO: 719) | | | | mitochondrial [305] | |
| QLYNIYAK(ac)HTK | 0.72 | NA | 0.00 | Putative ATP-dependent Clp | CLPP |
| (SEQ ID NO: 720) | | | | protease proteolytic subunit | |
| | | | | mitochondrial [211] | |
| GKGDK(ac)AQIEK | 0.73 | 0.00 | 0.00 | 60 kDa heat shock protein | HSPD1 |
| (SEQ ID NO: 721) | | | | mitochondrial [364] | |
| K(ac)FLVHNVK (SEQ ID NO: 722) | 0.73 | 0.00 | 0.00 | 60S ribosomal protein L32 [76] | RPL32 |
| GLIK(ac)LVSK (SEQ ID NO: 723) | 0.73 | NA | NA | 40S ribosomal protein S25 [98] | RPS25 |
| IGFAEK(ac)VAAK | 0.73 | 0.00 | NA | Isoform Mitochondrial of Fumarate | FH |
| (SEQ ID NO: 724) | | | | hydratase mitochondrial [292] | |
| (ac)AEK(ac)FDCHYCR | 0.73 | 0.00 | 0.00 | four and a half LIM domains | FHL1 |
| (SEQ ID NO: 725) | | | | protein 1 isoform 5 [20] | |
| GALPLDTVTFYK(ac)VIPK | 0.73 | 0.00 | 0.00 | Endoplasmic reticulum resident | ERP29 |
| (SEQ ID NO: 726) | | | | protein 29 [48] | |
| HWGGNVLGPK(ac)SVAR | 0.73 | NA | NA | 60S ribosomal protein L7a [245] | RPL7A, |
| (SEQ ID NO: 727) | | | | | SNORD24 |
| TITMK(ac)QLLR (SEQ ID NO: 728) | 0.73 | NA | 0.00 | Branched-chain-amino-acid | BCAT2 |
| | | | | aminotransferase [281] | |
| SK(ac)FVLVK (SEQ ID NO: 729) | 0.74 | NA | 0.00 | Endoplasmic reticulum resident | ERP29 |
| | | | | protein 29 [54] | |
| LVEDTK(ac)HRPK | 0.74 | NA | NA | 39S ribosomal protein L9 | MRPL9 |
| (SEQ ID NO: 730) | | | | mitochondrial [87] | |
| TVGQLYK(ac)EQLAK | 0.75 | NA | 0.00 | Isoform 1 of Myosin-9 [651] | MYH9 |
| (SEQ ID NO: 731) | | | | | |
| AAETCQEPK(ac)EFR | 0.75 | 0.00 | 0.00 | NADH dehydrogenase [ubiquinone] | NDUFAF4 |
| (SEQ ID NO: 732) | | | | 1 alpha subcomplex | |
| | | | | assembly factor 4 [91] | |
| VVLIGDSGVGK(ac)SNLLSR | 0.76 | 0.00 | NA | Ras-related protein Rab-11B [24] | RAB11B |
| (SEQ ID NO: 733) | | | | | |
| SGK(ac)YVLGYK (SEQ ID NO: 734) | 0.76 | 0.00 | NA | 60S ribosomal protein L30 [26] | RPL30 |
| ILMEHIHK(ac)LK | 0.76 | 0.00 | 0.00 | 60S ribosomal protein L19 [144] | RPL19 |
| (SEQ ID NO: 735) | | | | | |
| EK(ac)VFYSLMR (SEQ ID NO: 736) | 0.78 | NA | NA | Epoxide hydrolase 1 [288] | EPHX1 |
| K(ac)FDQLLAEEK | 0.78 | NA | 0.00 | Isoform 1 of Myosin-9 [1445] | MYH9 |
| (SEQ ID NO: 737) | | | | | |
| FVEFDMK(ac)PVCK | 0.79 | NA | 0.00 | Isoform 2 of LIM and senescent | LIMS2 |
| (SEQ ID NO: 738) | | | | cell antigen-like-containing | |
| | | | | domain protein 2 [326] | |
| K(ac)GFITVDGR (SEQ ID NO: 739) | 0.79 | NA | NA | Laminin subunit alpha-1 [2808] | LAMA1 |
| KAEAQIAAK(ac)NLDK | 0.80 | 0.00 | 0.00 | Aspartate aminotransferase | GOT2 |
| (SEQ ID NO: 740) | | | | mitochondrial [90] | |
| DK(ac)QFVAK (SEQ ID NO: 741) | 0.80 | NA | NA | Staphylococcal nuclease domain- | SND1 |
| | | | | containing protein 1 [341] | |
| AGACNTVIK(ac)QLMR | 0.82 | 0.00 | NA | Isoform SERCA3B of | ATP2A3 |
| (SEQ ID NO: 742) | | | | Sarcoplasmic/endoplasmic | |
| | | | | reticulum calcium ATPase | |
| | | | | 3 [476] | |
| KQQAK(ac)FDECVLDK | 0.82 | NA | NA | NADH dehydrogenase [ubiquinone] | NDUFA8 |
| (SEQ ID NO: 743) | | | | 1 alpha subcomplex | |
| | | | | subunit 8 [106] | |
| ALCTGEK(ac)GFGYK | 0.82 | 0.00 | 0.00 | Peptidyl-prolyl cis-trans | PPIF |
| (SEQ ID NO: 744) | | | | isomerase F mitochondrial [86] | |
| IYK(ac)SDGIK (SEQ ID NO: 745) | 0.83 | 0.00 | 0.00 | ADP/ATP translocase 2 [166] | SLC25A5 |
| DEGGK(ac)AFFK (SEQ ID NO: 746) | 0.84 | 0.00 | NA | ADP/ATP translocase 2 [268] | SLC25A5 |
| SYLK(ac)EFR (SEQ ID NO: 747) | 0.85 | NA | NA | Isoform 1 of Trans-23-enoyl-CoA | TECR |
| | | | | reductase [291] | |
| VPK(ac)TAENFR (SEQ ID NO: 748) | 0.85 | 0.00 | 0.00 | Peptidyl-prolyl cis-trans | PPID |
| | | | | isomerase D [43] | |
| VNCLDK(ac)FWHK | 0.85 | 0.00 | 0.00 | Isoform 1 of LIM and SH3 domain | LASP1 |
| (SEQ ID NO: 749) | | | | protein 1 [23] | |
| KGEDFVK(ac)TLK | 0.85 | NA | 0.00 | Malate dehydrogenase | MDH2 |
| (SEQ ID NO: 750) | | | | mitochondrial [335] | |
| EAEFTK(ac)SIAK | 0.86 | 0.00 | 0.00 | Signal peptidase complex | LOC653566, |
| (SEQ ID NO: 751) | | | | subunit 2 [191] | SPCS2 |
| SGYYK(ac)VLGK (SEQ ID NO: 752) | 0.86 | NA | NA | 60S ribosomal protein L27a [110] | RPL27A |
| YVLGYK(ac)QTLK | 0.87 | 0.00 | NA | 60S ribosomal protein L30 [32] | RPL30 |
| (SEQ ID NO: 753) | | | | | |
| FEDK(ac)TVAYTEQK | 0.88 | NA | 0.00 | Protein disulfide-isomerase | PDIA3 |
| (SEQ ID NO: 754) | | | | A3 [218] | |
| IVNALK(ac)SQVDK | 0.88 | 0.00 | NA | Ornithine aminotransferase | OAT |
| (SEQ ID NO: 755) | | | | mitochondrial [102] | |
| ILEFFGLK(ac)K (SEQ ID NO: 756) | 0.89 | 0.00 | 0.00 | Protein disulfide-isomerase [308] | P4HB |
| LCGQDLNK(ac)TSR | 0.89 | NA | NA | Isoform 1 of UDP-N- | DPAGT1 |
| (SEQ ID NO: 757) | | | | acetylglucosamine--dolichyl- | |
| | | | | phosphate N- | |
| | | | | acetylglucosaminephospho- | |
| | | | | transferase [48] | |
| VYFK(ac)DTHPK (SEQ ID NO: 758) | 0.90 | NA | NA | Isoform 1 of NADH-cytochrome b5 | CYB5R3 |
| | | | | reductase 3 [115] | |
| ILSK(ac)LSEETK | 0.91 | 0.00 | NA | Isoform 1 of E3 UFM1-protein | UFL1 |
| (SEQ ID NO: 759) | | | | ligase 1 [614] | |
| ATWK(ac)SNYFLK | 0.91 | NA | NA | 60S acidic ribosomal protein | RPLP0 |
| (SEQ ID NO: 760) | | | | P0 [10] | |
| AVDSQILPK(ac)IK | 0.92 | NA | NA | 60S ribosomal protein L6 [261] | RPL6 |
| (SEQ ID NO: 761) | | | | | |
| EK(ac)LQEEMLQR | 0.92 | NA | NA | Vimentin [188] | VIM |
| (SEQ ID NO: 762) | | | | | |
| MIYASSK(ac)DAIKK | 0.93 | 0.00 | 0.00 | Cofilin-1 [121] | CFL1 |
| (SEQ ID NO: 763) | | | | | |
| YNDPIYVK(ac)LEK | 0.93 | NA | 0.00 | Isoform A of AP-1 complex | AP1B1 |
| (SEQ ID NO: 764) | | | | subunit beta-1 [335] | |
| VTDDLVCLVYK(ac)TDQAQDVK | 0.93 | 0.00 | 0.00 | Isoform 1 of Signal recognition | SRP9 |
| (SEQ ID NO: 765) | | | | particle 9 kDa protein [52] | |
| TVVNK(ac)DVFR (SEQ ID NO: 766) | 0.94 | 0.00 | NA | 60S ribosomal protein L27 [98] | RPL27 |
| NLQEAEEWYK(ac)SK | 0.94 | 0.00 | 0.00 | Isoform 1 of Peripherin [288] | PRPH |
| (SEQ ID NO: 767) | | | | | |
| K(ac)FGGPGAR (SEQ ID NO: 768) | 0.97 | NA | NA | 40S ribosomal protein S16 [131] | RPS16 |
| LLLLGAGESGK(ac)STIVK | 0.97 | 0.00 | 0.00 | Guanine nucleotide-binding | GNAL |
| (SEQ ID NO: 769) | | | | protein G(olf) subunit | |
| | | | | alpha [55] | |
| SFVK(ac)VYNYNHLMPTR | 0.99 | NA | 0.00 | 60S ribosomal protein | RPL27 |
| (SEQ ID NO: 770) | | | | L27 [73] | |
| YVK(ac)ALFR (SEQ ID NO: 771) | 0.99 | NA | 0.00 | Mitochondrial import receptor | TOMM70A |
| | | | | subunit TOM70 [188] | |
| LEPLK(ac)PLK (SEQ ID NO: 772) | 1.00 | 0.00 | 0.00 | Isoform 3 of Proline-rich | PRR12 |
| | | | | protein 12 [1223] | |
| VGDK(ac)VLLPEYGGTK | 1.00 | 0.00 | 0.00 | 10 kDa heat shock protein | HSPE1 |
| (SEQ ID NO: 773) | | | | mitochondrial [70] | |
| GGVVGIK(ac)VDK | 1.00 | NA | 0.00 | Fructose-bisphosphate aldolase | ALDOA |
| (SEQ ID NO: 774) | | | | A [108] | |
| VAFK(ac)YCQK (SEQ ID NO: 775) | 1.00 | NA | NA | Isoform 1 of Dolichyl-phosphate | ALG5 |
| | | | | beta-glucosyltransferase [122] | |
| SVPHLQK(ac)VFDR | 1.00 | NA | 0.00 | Putative annexin A2-like | ANXA2P2 |
| (SEQ ID NO: 776) | | | | protein [227] | |
| VYK(ac)EMYK (SEQ ID NO: 777) | 1.00 | NA | 0.00 | Putative annexin A2-like | ANXA2P2 |
| | | | | protein [148] | |
| SELTGK(ac)FEK (SEQ ID NO: 778) | 1.00 | NA | 0.00 | Annexin A5 [76] | ANXA5 |
| EFIEK(ac)YDK (SEQ ID NO: 779) | 1.00 | 0.00 | 0.00 | Annexin A6 [644] | ANXA6 |
| ESCSK(ac)HASGTSFHLPQPSFR | 1.00 | 0.00 | NA | Bromodomain and PHD finger- | BRPF3 |
| (SEQ ID NO: 780) | | | | containing protein 3 [105] | |
| KAEAAVVAVAEK(ac)R | 1.00 | NA | NA | Selenoprotein H [20] | C11orf31 |
| (SEQ ID NO: 781) | | | | | |
| VNK(ac)EVER (SEQ ID NO: 782) | 1.00 | NA | NA | Isoform Long of ES1 protein | C21orf33 |
| | | | | homolog mitochondrial [157] | |
| GFSIPECQK(ac)LLPK | 1.00 | NA | NA | Citrate synthase mitochondrial | CS |
| (SEQ ID NO: 783) | | | | [103] | |
| QNIILK(ac)NDK (SEQ ID NO: 784) | 1.00 | NA | NA | Uncharacterized protein | CXorf59 |
| | | | | CXorf59 [13] | |
| NEEATK(ac)HLECTK | 1.00 | NA | NA | Isoform 1 of Fragile X mental | FXR1 |
| (SEQ ID NO: 785) | | | | retardation syndrome-related | |
| | | | | protein 1 [207] | |
| AGK(ac)DSGK(ac)AK | 1.00 | NA | NA | Histone H2A.V [8:12] | H2AFV |
| (SEQ ID NO: 786) | | | | | |
| ESK(ac)ALMGLYHGQVLCK | 1.00 | NA | NA | Trifunctional enzyme subunit alpha | HADHA |
| (SEQ ID NO: 787) | | | | mitochondrial [337] | |
| FGAPQK(ac)DVK (SEQ ID NO: 788) | 1.00 | 0.00 | 0.00 | Trifunctional enzyme subunit alpha | HADHA |
| | | | | mitochondrial [359] | |
| ILQEGVDPK(ac)K | 1.00 | 0.00 | 0.00 | Trifunctional enzyme subunit alpha | HADHA |
| (SEQ ID NO: 789) | | | | mitochondrial [569] | |
| GSK(ac)K(ac)AITK | 1.00 | NA | NA | Histone H2B type 1-B [16:17] | HIST1H2BB |
| (SEQ ID NO: 790) | | | | | |
| PEPTK(ac)SAPAPK(ac)K | 1.00 | NA | NA | Histone H2B type 1-D [6:12] | HIST1H2BD |
| (SEQ ID NO: 791) | | | | | |
| GSK(ac)K(ac)AINK | 1.00 | 0.00 | NA | Histone H2B type 1-M [16:17] | HIST1H2BM |
| (SEQ ID NO: 792) | | | | | |
| K(ac)AINK(ac)AQK | 1.00 | 0.00 | 0.00 | Histone H2B type 1-M [17:21] | HIST1H2BM |
| (SEQ ID NO: 793) | | | | | |
| K(ac)AVTK(ac)AQK | 1.00 | NA | NA | Histone H2B type 2-E [17:21] | HIST2H2BE |
| (SEQ ID NO: 794) | | | | | |
| PEPAK(ac)SAPAPKK | 1.00 | NA | NA | Histone H2B type 2-E [6] | HIST2H2BE |
| (SEQ ID NO: 795) | | | | | |
| K(ac)QLATK(ac)AAR | 1.00 | 0.00 | NA | Histone H3.2 [19:24] | HIST2H3D, |
| (SEQ ID NO: 796) | | | | | HIST2H3A, |
| | | | | | HIST2H3C |
| TEMDWVLK(ac)HSGPNSADSANDGFVR | 1.00 | NA | 0.00 | Heterogeneous nuclear | HNRNPF |
| (SEQ ID NO: 797) | | | | ribonucleoprotein F [98] | |
| ADLIK(ac)QYGR (SEQ ID NO: 798) | 1.00 | 0.00 | NA | Inactive hydroxysteroid | HSDL1 |
| | | | | dehydrogenase-like protein 1 [64] | |
| FYEQFSK(ac)NIK | 1.00 | NA | NA | Isoform 2 of Heat shock protein | HSP90AA1 |
| (SEQ ID NO: 799) | | | | HSP 90-alpha [565] | |
| LMK(ac)ICMNEDPAK | 1.00 | NA | 0.00 | Integrin-linked protein kinase | ILK |
| (SEQ ID NO: 800) | | | | [426] | |
| (ac)ASPSLERPEK(ac)GAGK | 1.00 | 0.00 | NA | KRR1 small subunit processome | KRR1 |
| (SEQ ID NO: 801) | | | | component homolog [11] | |
| INKALDK(ac)TK (SEQ ID NO: 802) | 1.00 | NA | NA | Isoform 1 of Myosin-9 [439] | MYH9 |
| IANILK(ac)DKDPPIPVAK | 1.00 | NA | NA | Protein disulfide-isomerase | PDIA4 |
| (SEQ ID NO: 803) | | | | A4 [109] | |
| NTEDLQCYVK(ac)PTK | 1.00 | NA | NA | Protein Jade-3 [735] | PHF16 |
| (SEQ ID NO: 804) | | | | | |
| VDEVK(ac)STIK (SEQ ID NO: 805) | 1.00 | NA | 0.00 | 60S ribosomal protein L10a [152] | RPL10A |
| KLQK(ac)AALLK (SEQ ID NO: 806) | 1.00 | NA | NA | Ribosomal protein L14 | RPL14 |
| | | | | variant [132] | |
| YSVDIPLDK(ac)TVVNK | 1.00 | 0.00 | 0.00 | 60S ribosomal protein L27 [93] | RPL27 |
| (SEQ ID NO: 807) | | | | | |
| LNK(ac)AVWAK (SEQ ID NO: 808) | 1.00 | NA | 0.00 | 60S ribosomal protein L31 [70] | RPL31 |
| K(ac)FGQGSR (SEQ ID NO: 809) | 1.00 | NA | NA | 40S ribosomal protein S29 [13] | RPS29 |
| DQTK(ac)SIVEK (SEQ ID NO: 810) | 1.00 | 0.00 | NA | Isoform 3 of Reticulon-3 [217] | RTN3 |
| YMVK(ac)SCGK (SEQ ID NO: 811) | 1.00 | 0.00 | 0.00 | 60S ribosomal protein L10 [78] | SNORA70, |
| | | | | | RPL10 |
| LAHEVGWK(ac)YQAVTATLEEK | 1.00 | 0.00 | 0.00 | 60S ribosomal protein L13a [148] | SNORD32A, |
| (SEQ ID NO: 812) | | | | SNORD33, SNORD34 | RPL13A, |
| ALEK(ac)IDLK (SEQ ID NO: 813) | 1.00 | 0.00 | NA | 60S ribosomal protein L3 [362] | SNORD43, |
| | | | | | RPL3 |
| AMMEK(ac)LVK(ac)SISQLK | 1.00 | NA | NA | Isoform 1 of Signal | STAT2 |
| (SEQ ID NO: 814) | | | | transducer and activator of | |
| | | | | transcription 2 [158:161] | |
| LLGITK(ac)ECVMR | 1.00 | NA | NA | Talin-1 [334] | TLN1 |
| (SEQ ID NO: 815) | | | | | |
| GK(ac)GK(ac)AGR | 1.00 | NA | NA | Tubulin polymerization-promoting | TPPP |
| (SEQ ID NO: 816) | | | | protein [187:189] | |
| LGDVISIQPCPDVK(ac)YGK | 1.00 | NA | 0.00 | Transitional endoplasmic reticulum | VCP |
| (SEQ ID NO: 817) | | | | ATPase [109] | |

### Example 2

### CNP Analysis

This example describes the CNP analysis that was performed on Compound B treated cells from Example 1.

The SILAC method was performed as described in Example 1.

The harvested “heavy” and “light” labeled cells were initially lysed with 2×NETN buffer (200 mM NaCl, 100 mM Tris-Cl, 2 mM EDTA, 1.0% NP-40, pH 7.2) on ice for 15 min, respectively. The supernatants were saved after 20,000×g centrifugation for 10 minutes at 4° C. After measurement of the protein concentration in “heavy” or “light” labeled supernatants, equal amounts of crude proteins in supernatant were mixed, and the crude proteins were precipitated by adding trifluoroacetic acid (TFA) with 15% final concentration (v/v) (this was called the cytosolic fraction). After washing twice with −20° C. acetone, the proteins pellets were dissolved in 100 mM NH₄HCO₃ (pH 8.0) for trypsin digestion.

The remaining cell pellets were further lysed with 1×ETN buffer (100 mM NaCl, 50 mM Tris-Cl, 1 mM EDTA, pH 7.2) supplemented with 0.5% Triton X-100 on ice for 15 min, respectively. The resulting lysates were clarified by 20,000×g centrifugation for 10 minutes, and the supernatant was saved as nuclear extracts fractions. After measurement of the protein concentration in “heavy” or “light” labeled supernatant, equal amount of crude proteins in supernatant were mixed, and the crude proteins were precipitated by adding trifluoroacetic acid (TFA) with 15% final concentration (v/v) (this was called the nuclear extracts fraction). After washing twice with −20° C. acetone, the proteins pellets were dissolved in 100 mM NH₄HCO₃ (pH 8.0) for trypsin digestion.

The final remaining cell pellets were dissolved in 8 M urea to extract the chromatin-binding proteins. After measurement of protein concentration, equal amount of chromatin-binding proteins in urea solution were mixed, and the proteins were precipitated by adding trifluoroacetic acid (TFA) with 15% final concentration (v/v) (this was called the nuclear pellet fraction). After washing twice with −20° C. acetone, the proteins pellets were dissolved in 100 mM NH₄HCO₃ for trypsin digestion.

After trypsin digestion for the above three fractions, the Kac affinity enrichment followed by MS analysis and data inquiry were separately performed. The combined Kac data formed the total Kac profiling data in the pair of Compound B vs DMSO.

Compound B treated cell lysate was fractionated into three subcellular fractions—cytoplasm (C), soluble nuclear extract (N), and insoluble nuclear pellet (P), as described above. Any peptides identified as HDAC6 specific in Example 1 with this subcellular localization information was extremely helpful for understanding their functions inside cells.

The results of these studies are shown in Table 2.

The first column of Table 2 shows the amino acid sequence of the acetylated peptide that was identified by the SILAC method, wherein “(ac)” means that the previous amino acid was acetylated. This column is identical to the first column in Table 1.

The second column of Table 2 shows the gene symbol for the peptide in column 1 This column is identical to the sixth column in Table 1.

The third, fourth, and fifth columns of Table 2 show data for Compound B. A “+” indicates a 1.3 fold change or greater in the quantity of peptide when normalized to the total (C+N+P=total) for each peptide for each drug treated group of cells. A “−” indicates a 1.29 fold change or less in the quantity of peptide when normalized to the total (C+N+P=total) for each peptide for each drug treated group of cells. “None” indicates unknown.

The sixth, seventh, and eighth columns of Table 2 show data for Compound B. The numbers in these columns represent the fold change in the quantity of peptide when normalized to the total (C+N+P=total) for each peptide for each drug treated group of cells. The numbers are in a log 2 scale so 1 represents a 2× change, 0 represents no change, and None represents unknown.

TABLE 2

| Peptide | Gene | C | N | P | C | N | P |
|---|---|---|---|---|---|---|---|
| FK(ac)ESFAEMNR (SEQ ID NO: 1) | COX4I1 | − | + | − | NA | −0.72 | NA |
| ELNK(ac)ILEGR (SEQ ID NO: 2) | DLAT | + | − | − | 0.47 | NA | NA |
| AAEVLNK(ac)HSLSGRPLK (SEQ ID NO: 3) | HNRNPM | − | − | + | NA | NA | −0.74 |
| LYK(ac)EELEQTYHAK (SEQ ID NO: 4) | LMNB1 | − | − | + | NA | NA | 0.48 |
| TLVLSDK(ac)HSPQK(ac)K | MLL3 | − | + | + | 0.33 | 0.40 | 0.50 |
| (SEQ ID NO: 5) | | | | | | | |
| (ac)CNTPTYCDLGK(ac)AAK | VDAC3 | − | + | − | NA | −0.63 | NA |
| (SEQ ID NO: 6) | | | | | | | |
| LGTDESK(ac)FNAVLCSR (SEQ ID NO: 7) | ANXA11 | + | − | − | 0.50 | NA | NA |
| SFDSEFK(ac)LACK (SEQ ID NO: 8) | DYNC1H1 | + | − | − | 0.51 | NA | NA |
| EPVAVLK(ac)ANR (SEQ ID NO: 9) | HEXB | − | + | − | NA | −0.40 | NA |
| LNK(ac)DQWEER (SEQ ID NO: 10) | MSN | − | + | − | NA | 0.71 | NA |
| DFLAGGVAAAISK(ac)TAVAPIER | SLC25A5 | − | + | − | NA | −0.43 | NA |
| (SEQ ID NO: 11) | | | | | | | |
| YYNK(ac)YINVK (SEQ ID NO: 12) | ATP5J2 | + | − | + | −0.45 | 0.11 | −0.53 |
| TNK(ac)NK(ac)SSISR (SEQ ID NO: 13) | CREBBP | + | + | + | 0.62 | 0.63 | 0.59 |
| TSK(ac)NK(ac)SSLSR (SEQ ID NO: 14) | EP300 | + | + | − | 0.44 | 0.40 | NA |
| AGGK(ac)AGK(ac)DSGK (SEQ ID NO: 15) | H2AFV | − | − | + | NA | NA | 0.87 |
| PDPAK(ac)SAPAPK(ac)K | HIST1H2BO | + | + | + | 0.66 | 0.49 | 0.95 |
| (SEQ ID NO: 16) | | | | | | | |
| YLTNQK(ac)NSNSK(ac)NDR | MEAF6 | − | + | − | NA | 0.43 | NA |
| (SEQ ID NO: 17) | | | | | | | |
| GVTQFGNK(ac)YIQQTKPLTLER | NUDT21 | + | + | + | 0.50 | 0.50 | 0.50 |
| (SEQ ID NO: 18) | | | | | | | |
| IHNFGLIQEK(ac)LAR (SEQ ID NO: 19) | ACADVL | NONE | NONE | NONE | NONE | NONE | NONE |
| LDHLAEK(ac)FR (SEQ ID NO: 20) | ACTN1 | NONE | NONE | NONE | NONE | NONE | NONE |
| LK(ac)DISTLEPLKK (SEQ ID NO: 21) | ANP32B | NONE | NONE | NONE | NONE | NONE | NONE |
| QRQEVCQSYK(ac)SLYGK (SEQ ID NO: 22) | ANXA6 | NONE | NONE | NONE | NONE | NONE | NONE |
| MYFDK(ac)YVLKPATEGK (SEQ ID NO: 23) | ATAD3A | + | − | − | 0.41 | −0.30 | 0.20 |
| IVEYEK(ac)EMEK (SEQ ID NO: 24) | ATP5H | NONE | NONE | NONE | NONE | NONE | NONE |
| FYGDEEK(ac)DKGLQTSQDAR | CALR | NONE | NONE | NONE | NONE | NONE | NONE |
| (SEQ ID NO: 25) | | | | | | | |
| ALEHFTDLYDIK(ac)R (SEQ ID NO: 26) | CLTC | NONE | NONE | NONE | NONE | NONE | NONE |
| KYEFSPOTLCCYGK(ac)QLCTIPR | CREBBP | NONE | NONE | NONE | NONE | NONE | NONE |
| (SEQ ID NO: 27) | | | | | | | |
| ALK(ac)DLYCHK (SEQ ID NO: 28) | CREM | NONE | NONE | NONE | NONE | NONE | NONE |
| LYCPK(ac)CMDVYTPK (SEQ ID NO: 29) | CSNK2B | NONE | NONE | NONE | NONE | NONE | NONE |
| FK(ac)DPNCVGIVLASR (SEQ ID NO: 30) | DAZAP1 | NONE | NONE | NONE | NONE | NONE | NONE |
| APTIVGK(ac)SSLNPILFR | DDOST | NONE | NONE | NONE | NONE | NONE | NONE |
| (SEQ ID NO: 31) | | | | | | | |
| FEK(ac)NFYVEHPEVAR (SEQ ID NO: 32) | DDX17 | NONE | NONE | NONE | NONE | NONE | NONE |
| DWVLNEFK(ac)HGK (SEQ ID NO: 33) | DDX5 | NONE | NONE | NONE | NONE | NONE | NONE |
| FGNPGEK(ac)LVK (SEQ ID NO: 34) | DDX5 | NONE | NONE | NONE | NONE | NONE | NONE |
| VVK(ac)HPIFER (SEQ ID NO: 35) | DNAJB11 | NONE | NONE | NONE | NONE | NONE | NONE |
| TCEESSFCK(ac)R (SEQ ID NO: 36) | GANAB | NONE | NONE | NONE | NONE | NONE | NONE |
| ASGLAAGK(ac)GVIVAK (SEQ ID NO: 37) | GART | − | − | + | 0.20 | 0.17 | 0.43 |
| IIVDELK(ac)QEVISTSSK | GNB2L1 | NONE | NONE | NONE | NONE | NONE | NONE |
| (SEQ ID NO: 38) | | | | | | | |
| LVK(ac)VWNLANCK (SEQ ID NO: 39) | GNB2L1 | NONE | NONE | NONE | NONE | NONE | NONE |
| (ac)AHYNFK(ac)K (SEQ ID NO: 40) | GTPBP4 | NONE | NONE | NONE | NONE | NONE | NONE |
| GQQQVFK(ac)GLNDK (SEQ ID NO: 41) | HADHA | NONE | NONE | NONE | NONE | NONE | NONE |
| K(ac)HPDSSVNFAEFSK (SEQ ID NO: 42) | HMGB2 | NONE | NONE | NONE | NONE | NONE | NONE |
| RGEEGHDPK(ac)EPEQLR (SEQ ID NO: 43) | HNRNPA3 | NONE | NONE | NONE | NONE | NONE | NONE |
| AFITNIPFDVK(ac)WQSLK | HNRNPM | NONE | NONE | NONE | NONE | NONE | NONE |
| (SEQ ID NO: 44) | | | | | | | |
| VK(ac)VLFVR (SEQ ID NO: 45) | HNRNPR | NONE | NONE | NONE | NONE | NONE | NONE |
| GYFEYIEENK(ac)YSR (SEQ ID NO: 46) | HNRNPU | NONE | NONE | NONE | NONE | NONE | NONE |
| TTWVTK(ac)HAAENPGK (SEQ ID NO: 47) | HNRNPU | NONE | NONE | NONE | NONE | NONE | NONE |
| EELVK(ac)NLGTIAK (SEQ ID NO: 48) | HSP90B1 | NONE | NONE | NONE | NONE | NONE | NONE |
| ELISNASDALDK(ac)IR (SEQ ID NO: 49) | HSP90B1 | NONE | NONE | NONE | NONE | NONE | NONE |
| ETLQQHK(ac)LLK (SEQ ID NO: 50) | HSP90B1 | NONE | NONE | NONE | NONE | NONE | NONE |
| IADDK(ac)YNDTFWK (SEQ ID NO: 51) | HSP90B1 | NONE | NONE | NONE | NONE | NONE | NONE |
| NK(ac)EIFLR (SEQ ID NO: 52) | HSP90B1 | − | − | + | 0.01 | 0.04 | 0.41 |
| IINEPTAAAIAYGLDK(ac)R | HSPA5 | NONE | NONE | NONE | NONE | NONE | NONE |
| (SEQ ID NO: 53) | | | | | | | |
| NQLTSNPENTVFDAK(ac)R | HSPA5 | NONE | NONE | NONE | NONE | NONE | NONE |
| (SEQ ID NO: 54) | | | | | | | |
| NQVAMNPTNTVFDAK(ac)R | HSPA8 | NONE | NONE | NONE | NONE | NONE | NONE |
| (SEQ ID NO: 55) | | | | | | | |
| YAASSYLSLTPEQWK(ac)SHR | IGLV2-14, | NONE | NONE | NONE | NONE | NONE | NONE |
| (SEQ ID NO: 56) | IGLC2 | | | | | | |
| IHLEIK(ac)QLNR (SEQ ID NO: 57) | LMAN1 | NONE | NONE | NONE | NONE | NONE | NONE |
| LYKEELEQTYHAK(ac)LENAR | LMNB1 | NONE | NONE | NONE | NONE | NONE | NONE |
| (SEQ ID NO: 58) | | | | | | | |
| GFGYK(ac)GSCFHR (SEQ ID NO: 59) | LOC390956 | NONE | NONE | NONE | NONE | NONE | NONE |
| ICCQFDFK(ac)R (SEQ ID NO: 60) | MAN2A1 | NONE | NONE | NONE | NONE | NONE | NONE |
| ADKDYHFK(ac)VDNDENEHQLSLR | NPM1 | NONE | NONE | NONE | NONE | NONE | NONE |
| (SEQ ID NO: 61) | | | | | | | |
| FINYVK(ac)NCFR (SEQ ID NO: 62) | NPM1 | NONE | NONE | NONE | NONE | NONE | NONE |
| SGVGNVFIK(ac)NLDK (SEQ ID NO: 63) | PABPC4 | NONE | NONE | NONE | NONE | NONE | NONE |
| GEK(ac)FVMQEEFSR (SEQ ID NO: 64) | PDIA3 | NONE | NONE | NONE | NONE | NONE | NONE |
| PSHLTNK(ac)FEDK (SEQ ID NO: 65) | PDIA3 | NONE | NONE | NONE | NONE | NONE | NONE |
| YGVSGYPTLK(ac)IFR (SEQ ID NO: 66) | PDIA3 | NONE | NONE | NONE | NONE | NONE | NONE |
| SLLGK(ac)DVLFLK (SEQ ID NO: 67) | PGK1 | NONE | NONE | NONE | NONE | NONE | NONE |
| SIYGEK(ac)FEDENFILK (SEQ ID NO: 68) | PPIA | NONE | NONE | NONE | NONE | NONE | NONE |
| TVPK(ac)TVDNFVALATGEK | PPIB | NONE | NONE | NONE | NONE | NONE | NONE |
| (SEQ ID NO: 69) | | | | | | | |
| VIFGLFGK(ac)TVPK (SEQ ID NO: 70) | PPIB | NONE | NONE | NONE | NONE | NONE | NONE |
| VIK(ac)DFMIQGGDFTR (SEQ ID NO: 71) | PPIB | NONE | NONE | NONE | NONE | NONE | NONE |
| IYGFYDECK(ac)R (SEQ ID NO: 72) | PPP1CC | NONE | NONE | NONE | NONE | NONE | NONE |
| EIDSVK(ac)YLECSALTQR | RAC2 | NONE | NONE | NONE | NONE | NONE | NONE |
| (SEQ ID NO: 73) | | | | | | | |
| (ac)ADK(ac)EAAFDDAVEER | RBBP4 | NONE | NONE | NONE | NONE | NONE | NONE |
| (SEQ ID NO: 74) | | | | | | | |
| ACFYNLDK(ac)FR (SEQ ID NO: 75) | RBM17 | NONE | NONE | NONE | NONE | NONE | NONE |
| LGLGEGAEEK(ac)SIPTLISR | RCC1 | NONE | NONE | NONE | NONE | NONE | NONE |
| (SEQ ID NO: 76) | | | | | | | |
| TGCIGAK(ac)HR (SEQ ID NO: 77) | RPL11 | NONE | NONE | NONE | NONE | NONE | NONE |
| WFQQK(ac)YDGIILPGK (SEQ ID NO: 78) | RPL11 | NONE | NONE | NONE | NONE | NONE | NONE |
| IFAPNHVVAK(ac)SR (SEQ ID NO: 79) | RPL18A | NONE | NONE | NONE | NONE | NONE | NONE |
| IEHIK(ac)HSK (SEQ ID NO: 80) | RPL21, | NONE | NONE | NONE | NONE | NONE | NONE |
| | SNORD102, | | | | | | |
| | SNORA27, | | | | | | |
| | RPL21P19 | | | | | | |
| YLK(ac)YLTK (SEQ ID NO: 81) | RPL22 | NONE | NONE | NONE | NONE | NONE | NONE |
| INFDK(ac)YHPGYFGK (SEQ ID NO: 82) | RPL27A | NONE | NONE | NONE | NONE | NONE | NONE |
| FIDTTSK(ac)FGHGR (SEQ ID NO: 83) | RPL3L | NONE | NONE | NONE | NONE | NONE | NONE |
| AASLK(ac)SNYNLPMHK (SEQ ID NO: 84) | RPL4 | NONE | NONE | NONE | NONE | NONE | NONE |
| LNILK(ac)LAPGGHVGR (SEQ ID NO: 85) | RPL4 | NONE | NONE | NONE | NONE | NONE | NONE |
| NFLGEK(ac)YIR (SEQ ID NO: 86) | RPL9 | NONE | NONE | NONE | NONE | NONE | NONE |
| VANVSLLALYK(ac)GK (SEQ ID NO: 87) | RPS23 | NONE | NONE | NONE | NONE | NONE | NONE |
| LAVLK(ac)YYK (SEQ ID NO: 88) | RPS27A | − | + | − | 0.03 | 0.68 | 0.01 |
| LK(ac)YALTGDEVKK (SEQ ID NO: 89) | RPS4X | NONE | NONE | NONE | NONE | NONE | NONE |
| SFQK(ac)IQVR (SEQ ID NO: 90) | RPS7 | NONE | NONE | NONE | NONE | NONE | NONE |
| ELLTLDEK(ac)DPR (SEQ ID NO: 91) | RPS9 | NONE | NONE | NONE | NONE | NONE | NONE |
| LQTQVFK(ac)LGLAK (SEQ ID NO: 92) | RPS9 | NONE | NONE | NONE | NONE | NONE | NONE |
| (ac)ATETVELHK(ac)LK (SEQ ID NO: 93) | SARNP | NONE | NONE | NONE | NONE | NONE | NONE |
| LAELK(ac)QECLAR (SEQ ID NO: 94) | SARNP | NONE | NONE | NONE | NONE | NONE | NONE |
| ANLVFK(ac)EIEK (SEQ ID NO: 95) | SCP2 | NONE | NONE | NONE | NONE | NONE | NONE |
| EGQNWLEK(ac)K (SEQ ID NO: 96) | SEC11C | NONE | NONE | NONE | NONE | NONE | NONE |
| GFGFIK(ac)LESR (SEQ ID NO: 97) | SFPQ | NONE | NONE | NONE | NONE | NONE | NONE |
| LFAK(ac)YGEPGEVFINK (SEQ ID NO: 98) | SFPQ | NONE | NONE | NONE | NONE | NONE | NONE |
| YGEPGEVFINK(ac)GK (SEQ ID NO: 99) | SFPQ | NONE | NONE | NONE | NONE | NONE | NONE |
| YIYDSAFHPDTGEK(ac)MILIGR | SFXN1 | NONE | NONE | NONE | NONE | NONE | NONE |
| (SEQ ID NO: 100) | | | | | | | |
| LVESLPQEIK(ac)ANVAK (SEQ ID NO: 101) | SLC25A10 | NONE | NONE | NONE | NONE | NONE | NONE |
| AFFK(ac)GAWSNVLR (SEQ ID NO: 102) | SLC25A5 | NONE | NONE | NONE | NONE | NONE | NONE |
| YFPTQALNFAFK(ac)DK (SEQ ID NO: 103) | SLC25A5 | NONE | NONE | NONE | NONE | NONE | NONE |
| TWEK(ac)LLLAAR (SEQ ID NO: 104) | SNORA6, | NONE | NONE | NONE | NONE | NONE | NONE |
| | RPSAP15, | | | | | | |
| | SNORA62, | | | | | | |
| | RPSA | | | | | | |
| YLMEEDEDAYK(ac)K (SEQ ID NO: 105) | SNORD21, | NONE | NONE | NONE | NONE | NONE | NONE |
| | RPL5 | | | | | | |
| TVFAEHISDECK(ac)R (SEQ ID NO: 106) | SNORD43, | NONE | NONE | NONE | NONE | NONE | NONE |
| | RPL3 | | | | | | |
| TKDIEDVFYK(ac)YGAIR (SEQ ID NO: 107) | SRSF1 | NONE | NONE | NONE | NONE | NONE | NONE |
| (ac)AEPSAPESK(ac)HK (SEQ ID NO: 108) | STT3B | NONE | NONE | NONE | NONE | NONE | NONE |
| INVYYNEATGGK(ac)YVPR | TUBB2C | NONE | NONE | NONE | NONE | NONE | NONE |
| (SEQ ID NO: 109) | | | | | | | |
| ILAEGGGAK(ac)FK (SEQ ID NO: 110) | TUFM | NONE | NONE | NONE | NONE | NONE | NONE |
| QIESK(ac)TAFQEALDAAGDK | TXN | NONE | NONE | NONE | NONE | NONE | NONE |
| (SEQ ID NO: 111) | | | | | | | |
| LFKPGQEAVK(ac)YQGPR | TXNDC5, | NONE | NONE | NONE | NONE | NONE | NONE |
| (SEQ ID NO: 112) | MUTED, | | | | | | |
| | MUTED- | | | | | | |
| | TXNDC5 | | | | | | |
| VYVAK(ac)VDCTAHSDVCSAQGVR | TXNDC5, | NONE | NONE | NONE | NONE | NONE | NONE |
| (SEQ ID NO: 113) | MUTED, | | | | | | |
| | MUTED- | | | | | | |
| | TXNDC5 | | | | | | |
| VVVTK(ac)YCDPDSYHR (SEQ ID NO: 114) | U2AF2 | NONE | NONE | NONE | NONE | NONE | NONE |
| FGIAAK(ac)YQIDPDACFSAK | VDAC1 | − | + | − | 0.21 | −0.68 | 0.28 |
| (SEQ ID NO: 115) | | | | | | | |
| YK(ac)WCEYGLTFTEK (SEQ ID NO: 116) | VDAC2 | − | + | + | NA | −0.70 | 0.47 |
| YK(ac)VCNYGLTFTQK (SEQ ID NO: 117) | VDAC3 | − | + | + | 0.17 | −0.89 | 0.62 |
| FANYIDK(ac)VR (SEQ ID NO: 118) | VIM | − | + | − | NA | −0.50 | −0.02 |
| ILLAELEQLK(ac)GQGK (SEQ ID NO: 119) | VIM | − | + | − | NA | −0.48 | 0.10 |
| LQDEIQNMK(ac)EEMAR (SEQ ID NO: 120) | VIM | − | + | − | NA | −0.50 | −0.08 |
| SK(ac)FADLSEAANR (SEQ ID NO: 121) | VIM | − | + | − | 0.01 | −0.47 | 0.08 |
| TNEK(ac)VELQELNDR (SEQ ID NO: 122) | VIM | − | + | − | NA | −0.62 | 0.15 |
| ALQEK(ac)VEIK (SEQ ID NO: 123) | XRCC5 | NONE | NONE | NONE | NONE | NONE | NONE |
| (ac)SIMSYNGGAIMAMK(ac)GK | — | NONE | NONE | NONE | NONE | NONE | NONE |
| (SEQ ID NO: 124) | | | | | | | |
| NLQK(ac)EVIHR (SEQ ID NO: 125) | ACOX1 | NONE | NONE | NONE | NONE | NONE | NONE |
| LVDQSGPPHEPK(ac)FVYQAK | ADAR | NONE | NONE | NONE | NONE | NONE | NONE |
| (SEQ ID NO: 126) | | | | | | | |
| HLSDSINQK(ac)HFEQAIER | AFG3L2 | NONE | NONE | NONE | NONE | NONE | NONE |
| (SEQ ID NO: 127) | | | | | | | |
| DAISGIGTDEK(ac)CLIEILASR | ANXA6 | NONE | NONE | NONE | NONE | NONE | NONE |
| (SEQ ID NO: 128) | | | | | | | |
| DLMTDLK(ac)SEISGDLAR | ANXA6 | NONE | NONE | NONE | NONE | NONE | NONE |
| (SEQ ID NO: 129) | | | | | | | |
| GTVRPANDFNPDADAK(ac)ALR | ANXA6 | NONE | NONE | NONE | NONE | NONE | NONE |
| (SEQ ID NO: 130) | | | | | | | |
| LENK(ac)MEGIGLK (SEQ ID NO: 131) | ARL6IP5 | NONE | NONE | NONE | NONE | NONE | NONE |
| EQEHMINWVEK(ac)HVVQSISTQQEK | ATP5F1 | NONE | NONE | NONE | NONE | NONE | NONE |
| (SEQ ID NO: 132) | | | | | | | |
| LNEQK(ac)LAQLEEAK (SEQ ID NO: 133) | ATP5F1 | NONE | NONE | NONE | NONE | NONE | NONE |
| YGPFVADFADK(ac)LNEQK | ATP5F1 | NONE | NONE | NONE | NONE | NONE | NONE |
| (SEQ ID NO: 134) | | | | | | | |
| SCAEWVSLSK(ac)AR (SEQ ID NO: 135) | ATP5H | NONE | NONE | NONE | NONE | NONE | NONE |
| VNLQNNPGAMEHFHMK(ac)LFR | BCAP31 | NONE | NONE | NONE | NONE | NONE | NONE |
| (SEQ ID NO: 136) | | | | | | | |
| LTALDYHNPAGFNCK(ac)DETEFR | C14orf166 | NONE | NONE | NONE | NONE | NONE | NONE |
| (SEQ ID NO: 137) | | | | | | | |
| IYFTDSSSK(ac)WQR (SEQ ID NO: 138) | C20orf3 | NONE | NONE | NONE | NONE | NONE | NONE |
| TPELNLDQFHDK(ac)TPYTIMFGPDK | CANX | NONE | NONE | NONE | NONE | NONE | NONE |
| (SEQ ID NO: 139) | | | | | | | |
| YIAK(ac)QESVEER (SEQ ID NO: 140) | CCDC144A | NONE | NONE | NONE | NONE | NONE | NONE |
| EDQK(ac)PVMDDQR (SEQ ID NO: 141) | CD74 | NONE | NONE | NONE | NONE | NONE | NONE |
| SHLIDK(ac)GMLTSTTEDE | CDS2 | NONE | NONE | NONE | NONE | NONE | NONE |
| (SEQ ID NO: 142) | | | | | | | |
| (ac)SVFGK(ac)LFGAGGGK | CHMP4B | NONE | NONE | NONE | NONE | NONE | NONE |
| (SEQ ID NO: 143) | | | | | | | |
| STTELPLTVSYDK(ac)VSLGR | CLPTM1L | NONE | NONE | NONE | NONE | NONE | NONE |
| (SEQ ID NO: 144) | | | | | | | |
| DCDHADEQK(ac)CYSCGEFGHIQK | CNBP | NONE | NONE | NONE | NONE | NONE | NONE |
| (SEQ ID NO: 145) | | | | | | | |
| GPIGSIGPK(ac)GIPGEDGYR | COL6A3 | NONE | NONE | NONE | NONE | NONE | NONE |
| (SEQ ID NO: 146) | | | | | | | |
| YLLYGEK(ac)GTGK (SEQ ID NO: 147) | DAP3 | NONE | NONE | NONE | NONE | NONE | NONE |
| NIDPK(ac)PCTPR (SEQ ID NO: 148) | DAZAP1 | − | + | − | 0.34 | 0.60 | 0.11 |
| FNQK(ac)GEVYK (SEQ ID NO: 149) | DBT | NONE | NONE | NONE | NONE | NONE | NONE |
| WVFK(ac)EEGVLR (SEQ ID NO: 150) | DDOST | − | + | − | NA | −0.90 | NA |
| SK(ac)EITVR (SEQ ID NO: 151) | DDX5 | NONE | NONE | NONE | NONE | NONE | NONE |
| K(ac)YEDICPSTHNMDVPNIK | EIF5A2 | NONE | NONE | NONE | NONE | NONE | NONE |
| (SEQ ID NO: 152) | | | | | | | |
| AHPVFYQGTYSQALNDAK(ac)R | FAF2 | NONE | NONE | NONE | NONE | NONE | NONE |
| (SEQ ID NO: 153) | | | | | | | |
| MPTPVIK(ac)AFGILK (SEQ ID NO: 154) | FH | NONE | NONE | NONE | NONE | NONE | NONE |
| SNFK(ac)TCEESSFCK (SEQ ID NO: 155) | GANAB | NONE | NONE | NONE | NONE | NONE | NONE |
| AVIWPQYVK(ac)DR (SEQ ID NO: 156) | GHITM | NONE | NONE | NONE | NONE | NONE | NONE |
| EAALEPSMEK(ac)IFK (SEQ ID NO: 157) | GHITM | − | + | + | NA | −0.54 | 0.52 |
| TAMK(ac)YNLGLDLR (SEQ ID NO: 158) | GLUD1 | NONE | NONE | NONE | NONE | NONE | NONE |
| NLDK(ac)EYLPIGGLAEFCK | GOT2 | NONE | NONE | NONE | NONE | NONE | NONE |
| (SEQ ID NO: 159) | | | | | | | |
| DSGNK(ac)PPGLLPR (SEQ ID NO: 160) | GSTK1 | NONE | NONE | NONE | NONE | NONE | NONE |
| GDASK(ac)EDIDTAMK (SEQ ID NO: 161) | HADH | NONE | NONE | NONE | NONE | NONE | NONE |
| GFGGK(ac)YGVER (SEQ ID NO: 162) | HCLS1 | + | − | − | 0.39 | 0.30 | 0.35 |
| YLENGK(ac)ETLQR (SEQ ID NO: 163) | HLA-H | NONE | NONE | NONE | NONE | NONE | NONE |
| K(ac)HPDASVNFSEFSK (SEQ ID NO: 164) | HMGB1P4 | NONE | NONE | NONE | NONE | NONE | NONE |
| GFAFVTFDDHDSVDK(ac)IVIQK | HNRNPA1 | NONE | NONE | NONE | NONE | NONE | NONE |
| (SEQ ID NO: 165) | | | | | | | |
| IFVGGLSPDTPEEK(ac)IR | HNRNPD | NONE | NONE | NONE | NONE | NONE | NONE |
| (SEQ ID NO: 166) | | | | | | | |
| ISK(ac)EVLAGR (SEQ ID NO: 167) | HNRNPU | NONE | NONE | NONE | NONE | NONE | NONE |
| VTEK(ac)IPVR (SEQ ID NO: 168) | HNRNPU | NONE | NONE | NONE | NONE | NONE | NONE |
| MKENQK(ac)HIYYITGETK | HSP90AA1 | NONE | NONE | NONE | NONE | NONE | NONE |
| (SEQ ID NO: 169) | | | | | | | |
| AFYK(ac)SFSK (SEQ ID NO: 170) | HSP90B1 | NONE | NONE | NONE | NONE | NONE | NONE |
| K(ac)TFEINPR (SEQ ID NO: 171) | HSP90B1 | NONE | NONE | NONE | NONE | NONE | NONE |
| AVEEK(ac)IEWLESHODADIEDFK | HSPA5 | NONE | NONE | NONE | NONE | NONE | NONE |
| (SEQ ID NO: 172) | | | | | | | |
| DVK(ac)FGADAR (SEQ ID NO: 173) | HSPD1 | NONE | NONE | NONE | NONE | NONE | NONE |
| IGIEIIK(ac)R (SEQ ID NO: 174) | HSPD1 | NONE | NONE | NONE | NONE | NONE | NONE |
| TLNDELEIIEGMK(ac)FDR | HSPD1 | NONE | NONE | NONE | NONE | NONE | NONE |
| (SEQ ID NO: 175) | | | | | | | |
| TPVIVTLK(ac)ENER (SEQ ID NO: 176) | HYOU1 | NONE | NONE | NONE | NONE | NONE | NONE |
| NTILK(ac)AYDGR (SEQ ID NO: 177) | IDH2 | NONE | NONE | NONE | NONE | NONE | NONE |
| HK(ac)DIDILIVR (SEQ ID NO: 178) | IDH3G | NONE | NONE | NONE | NONE | NONE | NONE |
| FEK(ac)EQQQLNWK (SEQ ID NO: 179) | ITGB7 | NONE | NONE | NONE | NONE | NONE | NONE |
| IGFGSFVDK(ac)TVLPFVSTVPSK | ITGB7 | NONE | NONE | NONE | NONE | NONE | NONE |
| (SEQ ID NO: 180) | | | | | | | |
| EVHK(ac)QVVESAYEVIK (SEQ ID NO: 181) | LDHA | NONE | NONE | NONE | NONE | NONE | NONE |
| DK(ac)TQYIFNNMVLK (SEQ ID NO: 182) | MAN2A1 | NONE | NONE | NONE | NONE | NONE | NONE |
| VALEIFQHSK(ac)YK (SEQ ID NO: 183) | MRPL39 | NONE | NONE | NONE | NONE | NONE | NONE |
| DELGDYLK(ac)PK (SEQ ID NO: 184) | MRPL46 | NONE | NONE | NONE | NONE | NONE | NONE |
| DAAGSGDK(ac)PGADTGR (SEQ ID NO: 185) | MRPL53 | NONE | NONE | NONE | NONE | NONE | NONE |
| VLK(ac)GNTAEGCVHETQEK | MYBBP1A | NONE | NONE | NONE | NONE | NONE | NONE |
| (SEQ ID NO: 186) | | | | | | | |
| FWNK(ac)FLENK (SEQ ID NO: 187) | NDUFB6 | − | − | + | NA | −0.10 | 0.58 |
| TYGEIFEK(ac)FHPIR (SEQ ID NO: 188) | NDUFC2 | NONE | NONE | NONE | NONE | NONE | NONE |
| PGGPALWGDAFK(ac)R (SEQ ID NO: 189) | NIPSNAP3A | NONE | NONE | NONE | NONE | NONE | NONE |
| QGFNVVVESGAGEASK(ac)FSDDHYR | NNT | NONE | NONE | NONE | NONE | NONE | NONE |
| (SEQ ID NO: 190) | | | | | | | |
| ADK(ac)DYHFK (SEQ ID NO: 191) | NPM1 | NONE | NONE | NONE | NONE | NONE | NONE |
| NYEHLFK(ac)VNDK (SEQ ID NO: 192) | PAM16 | NONE | NONE | NONE | NONE | NONE | NONE |
| VVVGK(ac)TFDSIVMDPK (SEQ ID NO: 193) | PDIA4 | NONE | NONE | NONE | NONE | NONE | NONE |
| NATVGQSVLNIK(ac)YK (SEQ ID NO: 194) | PEX2 | NONE | NONE | NONE | NONE | NONE | NONE |
| VLPSIVNEVLK(ac)SVVAK | PHB2 | NONE | NONE | NONE | NONE | NONE | NONE |
| (SEQ ID NO: 195) | | | | | | | |
| GDGTGGK(ac)SIYGER (SEQ ID NO: 196) | PPIB | NONE | NONE | NONE | NONE | NONE | NONE |
| GLFIIDDK(ac)GILR (SEQ ID NO: 197) | PRDX1 | NONE | NONE | NONE | NONE | NONE | NONE |
| KLPK(ac)NEPQNATGAPGR | RAB5C | NONE | NONE | NONE | NONE | NONE | NONE |
| (SEQ ID NO: 198) | | | | | | | |
| AGYNVK(ac)QLFR (SEQ ID NO: 199) | RAB6A | NONE | NONE | NONE | NONE | NONE | NONE |
| FLNAENAQK(ac)FK (SEQ ID NO: 200) | RANBP1 | NONE | NONE | NONE | NONE | NONE | NONE |
| NCMTDLLAK(ac)LEAK (SEQ ID NO: 201) | REEP5 | NONE | NONE | NONE | NONE | NONE | NONE |
| ADINTK(ac)WAATR (SEQ ID NO: 202) | RPL14 | NONE | NONE | NONE | NONE | NONE | NONE |
| RPAVK(ac)QFHDSK (SEQ ID NO: 203) | RPL18A | NONE | NONE | NONE | NONE | NONE | NONE |
| AYGGSMCAK(ac)CVR (SEQ ID NO: 204) | RPL34 | NONE | NONE | NONE | NONE | NONE | NONE |
| NVTLPAVFK(ac)APIRPDIVNFVHTNLR | RPL4 | NONE | NONE | NONE | NONE | NONE | NONE |
| (SEQ ID NO: 205) | | | | | | | |
| AGVNTVTTLVENK(ac)K (SEQ ID NO: 206) | RPL7A, | NONE | NONE | NONE | NONE | NONE | NONE |
| | SNORD24 | | | | | | |
| GIVK(ac)DIIHDPGR (SEQ ID NO: 207) | RPL8 | NONE | NONE | NONE | NONE | NONE | NONE |
| GHLENNPALEK(ac)LLPHIR | RPLP0 | NONE | NONE | NONE | NONE | NONE | NONE |
| (SEQ ID NO: 208) | | | | | | | |
| FFTVK(ac)LPVALDPGAK (SEQ ID NO: 209) | RPN1 | NONE | NONE | NONE | NONE | NONE | NONE |
| NVESYTK(ac)LGNPTR (SEQ ID NO: 210) | RPN1 | NONE | NONE | NONE | NONE | NONE | NONE |
| DLEK(ac)WQNNLLPSR (SEQ ID NO: 211) | RPS15A | NONE | NONE | NONE | NONE | NONE | NONE |
| IQDK(ac)EGIPPDQQR (SEQ ID NO: 212) | RPS27A | NONE | NONE | NONE | NONE | NONE | NONE |
| LIYDTK(ac)GR (SEQ ID NO: 213) | RPS4X | NONE | NONE | NONE | NONE | NONE | NONE |
| NKEEAAEYAK(ac)LLAK (SEQ ID NO: 214) | RPS6 | NONE | NONE | NONE | NONE | NONE | NONE |
| LIK(ac)VHLDK (SEQ ID NO: 215) | RPS7 | NONE | NONE | NONE | NONE | NONE | NONE |
| GYLTK(ac)EDLR (SEQ ID NO: 216) | S100A10 | NONE | NONE | NONE | NONE | NONE | NONE |
| IMK(ac)DLDQCR (SEQ ID NO: 217) | S100A10 | NONE | NONE | NONE | NONE | NONE | NONE |
| FLTK(ac)GDNNAVDDR (SEQ ID NO: 218) | SEC11B | NONE | NONE | NONE | NONE | NONE | NONE |
| FLEVIKPFCVILPEIQK(ac)PER | SEC61A1 | NONE | NONE | NONE | NONE | NONE | NONE |
| (SEQ ID NO: 219) | | | | | | | |
| GLSSLLYGSIPK(ac)AAVR | SLC25A1 | NONE | NONE | NONE | NONE | NONE | NONE |
| (SEQ ID NO: 220) | | | | | | | |
| LTEAKPVDK(ac)VK (SEQ ID NO: 221) | SLC25A10 | NONE | NONE | NONE | NONE | NONE | NONE |
| SLEK(ac)VCADLIR (SEQ ID NO: 222) | SNORD54, | NONE | NONE | NONE | NONE | NONE | NONE |
| | RPS20 | | | | | | |
| EVQINDLK(ac)EVVNK (SEQ ID NO: 223) | SNORD73A, | NONE | NONE | NONE | NONE | NONE | NONE |
| | RPS3A | | | | | | |
| (ac)SIGVPIK(ac)VLHEAEGHIVTCETNTGEVYR | SNRPD3 | NONE | NONE | NONE | NONE | NONE | NONE |
| (SEQ ID NO: 224) | | | | | | | |
| AVK(ac)HAEELER (SEQ ID NO: 225) | SRP68 | NONE | NONE | NONE | NONE | NONE | NONE |
| LSLDK(ac)VFR (SEQ ID NO: 226) | STOML2 | NONE | NONE | NONE | NONE | NONE | NONE |
| HIK(ac)ENDYYTPTGEFR (SEQ ID NO: 227) | STT3A | NONE | NONE | NONE | NONE | NONE | NONE |
| IGGSTDTGK(ac)HIK (SEQ ID NO: 228) | STT3A | NONE | NONE | NONE | NONE | NONE | NONE |
| LFVGSIPK(ac)SK (SEQ ID NO: 229) | SYNCRIP | NONE | NONE | NONE | NONE | NONE | NONE |
| HYEVEILDAK(ac)TR (SEQ ID NO: 230) | TECR | NONE | NONE | NONE | NONE | NONE | NONE |
| NAVQALIDK(ac)HQR (SEQ ID NO: 231) | TMEM68 | NONE | NONE | NONE | NONE | NONE | NONE |
| HVFTTFYAK(ac)TK (SEQ ID NO: 232) | TMEM70 | NONE | NONE | NONE | NONE | NONE | NONE |
| SFEEK(ac)VENLK (SEQ ID NO: 233) | TPD52 | NONE | NONE | NONE | NONE | NONE | NONE |
| SDCK(ac)EFSSEAR (SEQ ID NO: 234) | TRAP1 | NONE | NONE | NONE | NONE | NONE | NONE |
| SVQK(ac)LLDAVDTYIPVPAR | TUFM | NONE | NONE | NONE | NONE | NONE | NONE |
| (SEQ ID NO: 235) | | | | | | | |
| IGK(ac)VDCTQHYELCSGNQVR | TXNDC5, | NONE | NONE | NONE | NONE | NONE | NONE |
| (SEQ ID NO: 236) | MUTED, | | | | | | |
| | MUTED- | | | | | | |
| | TXNDC5 | | | | | | |
| NK(ac)TEDLEATSEHFK (SEQ ID NO: 237) | VAMP8 | NONE | NONE | NONE | NONE | NONE | NONE |
| FLK(ac)FGMTPSK (SEQ ID NO: 238) | VCP | NONE | NONE | NONE | NONE | NONE | NONE |
| DIFNK(ac)GFGFGLVK (SEQ ID NO: 239) | VDAC2 | NONE | NONE | NONE | NONE | NONE | NONE |
| GFGFGLVK(ac)LDVK (SEQ ID NO: 240) | VDAC2 | NONE | NONE | NONE | NONE | NONE | NONE |
| FLEQQNK(ac)ILLAELEQLK | VIM | NONE | NONE | NONE | NONE | NONE | NONE |
| (SEQ ID NO: 241) | | | | | | | |
| KVESLQEEIAFLK(ac)K (SEQ ID NO: 242) | VIM | NONE | NONE | NONE | NONE | NONE | NONE |
| FPTAIFESK(ac)GFR (SEQ ID NO: 243) | WHSC1L1 | NONE | NONE | NONE | NONE | NONE | NONE |
| LHTGEK(ac)PYK (SEQ ID NO: 244) | ZSCAN21 | NONE | NONE | NONE | NONE | NONE | NONE |
| KTPCGEGSK(ac)TWDR (SEQ ID NO: 245) | SNORD54, | NONE | NONE | NONE | NONE | NONE | NONE |
| | RPS20 | | | | | | |
| K(ac)YSQFINFPIYVWSSK | HSP90B1 | + | − | − | −0.43 | 0.32 | NA |
| (SEQ ID NO: 246) | | | | | | | |
| PLISVYSEK(ac)GESSGK (SEQ ID NO: 247) | RPL4 | NONE | NONE | NONE | NONE | NONE | NONE |
| ALDLNLK(ac)HQILPK (SEQ ID NO: 248) | UQCRB | NONE | NONE | NONE | NONE | NONE | NONE |
| IGQGYLIK(ac)DGK (SEQ ID NO: 249) | SNORD43, | NONE | NONE | NONE | NONE | NONE | NONE |
| | RPL3 | | | | | | |
| VGIVGK(ac)YGTR (SEQ ID NO: 250) | RPL37A | NONE | NONE | NONE | NONE | NONE | NONE |
| LYATMEK(ac)HK (SEQ ID NO: 251) | EP300 | NONE | NONE | NONE | NONE | NONE | NONE |
| TLQYK(ac)LLEPVLLLGK (SEQ ID NO: 252) | RPS16 | NONE | NONE | NONE | NONE | NONE | NONE |
| FPHSAHQK(ac)YVR (SEQ ID NO: 253) | RPL14 | NONE | NONE | NONE | NONE | NONE | NONE |
| YNCDK(ac)MICR (SEQ ID NO: 254) | UBA52 | NONE | NONE | NONE | NONE | NONE | NONE |
| SK(ac)SDPIMLLK (SEQ ID NO: 255) | PDHA1 | NONE | NONE | NONE | NONE | NONE | NONE |
| NICSK(ac)YSVR (SEQ ID NO: 256) | TXNDC5, | NONE | NONE | NONE | NONE | NONE | NONE |
| | MUTED, | | | | | | |
| | MUTED- | | | | | | |
| | TXNDC5 | | | | | | |
| NVLINK(ac)DIR (SEQ ID NO: 257) | CALR | NONE | NONE | NONE | NONE | NONE | NONE |
| YQQFK(ac)DFQR (SEQ ID NO: 258) | SNORD84, | NONE | NONE | NONE | NONE | NONE | NONE |
| | DDX39B | | | | | | |
| IPK(ac)HLTDAYFK (SEQ ID NO: 259) | RPL6 | NONE | NONE | NONE | NONE | NONE | NONE |
| AGLQVYNK(ac)CWK (SEQ ID NO: 260) | CD59 | NONE | NONE | NONE | NONE | NONE | NONE |
| EVEEDEYK(ac)AFYK (SEQ ID NO: 261) | HSP90B1 | NONE | NONE | NONE | NONE | NONE | NONE |
| AVLK(ac)FAAATGATPIAGR | SNORA6, | NONE | NONE | NONE | NONE | NONE | NONE |
| (SEQ ID NO: 262) | RPSAP15, | | | | | | |
| | SNORA62, | | | | | | |
| | RPSA | | | | | | |
| FVVEK(ac)AEQQK (SEQ ID NO: 263) | PHB | NONE | NONE | NONE | NONE | NONE | NONE |
| IQEVLK(ac)HAR (SEQ ID NO: 264) | MRPL20 | + | − | − | −0.65 | −0.09 | NA |
| GSK(ac)FYGPAGPYGIFAGR | PGRMC2 | NONE | NONE | NONE | NONE | NONE | NONE |
| (SEQ ID NO: 265) | | | | | | | |
| AK(ac)FEELNMDLFR (SEQ ID NO: 266) | HSPA5 | NONE | NONE | NONE | NONE | NONE | NONE |
| NQVALNPQNTVFDAK(ac)R | HSPA1B, | NONE | NONE | NONE | NONE | NONE | NONE |
| (SEQ ID NO: 267) | HSPA1A | | | | | | |
| DGLQNEK(ac)NIVSTPVK (SEQ ID NO: 268) | HMGCL | NONE | NONE | NONE | NONE | NONE | NONE |
| TLLIK(ac)TVETR (SEQ ID NO: 269) | VIM | NONE | NONE | NONE | NONE | NONE | NONE |
| TKYEK(ac)SLYSMIK (SEQ ID NO: 270) | ANXA6 | NONE | NONE | NONE | NONE | NONE | NONE |
| QNK(ac)LEQVEK (SEQ ID NO: 271) | ATP5O | NONE | NONE | NONE | NONE | NONE | NONE |
| ATVPK(ac)TEIR (SEQ ID NO: 272) | RPS24 | NONE | NONE | NONE | NONE | NONE | NONE |
| VHVIFNYK(ac)GK (SEQ ID NO: 273) | CALR | NONE | NONE | NONE | NONE | NONE | NONE |
| YDGK(ac)WEVEEMK (SEQ ID NO: 274) | CANX | NONE | NONE | NONE | NONE | NONE | NONE |
| RMELK(ac)ADQLYK (SEQ ID NO: 275) | PDHA2 | NONE | NONE | NONE | NONE | NONE | NONE |
| VMEHFIK(ac)LYK (SEQ ID NO: 276) | HSPA5 | NONE | NONE | NONE | NONE | NONE | NONE |
| (ac)AAK(ac)VFESIGK (SEQ ID NO: 277) | PHB | NONE | NONE | NONE | NONE | NONE | NONE |
| GWLK(ac)SNVSDAVACISTR | TPI1, | NONE | NONE | NONE | NONE | NONE | NONE |
| (SEQ ID NO: 278) | TPI1P1 | | | | | | |
| IVNSAQTGSFK(ac)QLTVK | ATP5L | NONE | NONE | NONE | NONE | NONE | NONE |
| (SEQ ID NO: 279) | | | | | | | |
| IFK(ac)SDGLR (SEQ ID NO: 280) | SLC25A4 | NONE | NONE | NONE | NONE | NONE | NONE |
| FVLSSGK(ac)FYGDEEKDK | CALR | NONE | NONE | NONE | NONE | NONE | NONE |
| (SEQ ID NO: 281) | | | | | | | |
| AINEAYK(ac)EDYHK (SEQ ID NO: 282) | ANXA6 | NONE | NONE | NONE | NONE | NONE | NONE |
| (ac)AEYLASIFGTEK(ac)DK | U2AF1 | NONE | NONE | NONE | NONE | NONE | NONE |
| (SEQ ID NO: 283) | | | | | | | |
| AISPDK(ac)DNFYFDVK (SEQ ID NO: 284) | NNT | NONE | NONE | NONE | NONE | NONE | NONE |
| NDTSGEYK(ac)K (SEQ ID NO: 285) | ANXA6 | NONE | NONE | NONE | NONE | NONE | NONE |
| NFEDVAFDEK(ac)K (SEQ ID NO: 286) | P4HB | NONE | NONE | NONE | NONE | NONE | NONE |
| FK(ac)LITEDVQGK (SEQ ID NO: 287) | SNORD73A, | NONE | NONE | NONE | NONE | NONE | NONE |
| | RPS3A | | | | | | |
| EIFQNEK(ac)R (SEQ ID NO: 288) | NNT | NONE | NONE | NONE | NONE | NONE | NONE |
| TCDLVGEK(ac)GK (SEQ ID NO: 289) | M6PR | NONE | NONE | NONE | NONE | NONE | NONE |
| VPVPEDK(ac)YTAQVDAEEKEDVK | ATP5H | − | − | + | 0.14 | 0.30 | 0.58 |
| (SEQ ID NO: 290) | | | | | | | |
| ILMSK(ac)PVLSGGTGR (SEQ ID NO: 291) | ZMYND8 | NONE | NONE | NONE | NONE | NONE | NONE |
| SAYFAEK(ac)LYK (SEQ ID NO: 292) | ANXA4 | NONE | NONE | NONE | NONE | NONE | NONE |
| YFLDK(ac)TLTSR (SEQ ID NO: 293) | BCKDK | NONE | NONE | NONE | NONE | NONE | NONE |
| VLSK(ac)EFHLNESGDPSSK | SET | NONE | NONE | NONE | NONE | NONE | NONE |
| (SEQ ID NO: 294) | | | | | | | |
| SEVDMLK(ac)IR (SEQ ID NO: 295) | ANXA2P2 | NONE | NONE | NONE | NONE | NONE | NONE |
| LVAENK(ac)FGK (SEQ ID NO: 296) | HADH | NONE | NONE | NONE | NONE | NONE | NONE |
| THILLFLPK(ac)SVSDYDGK | P4HB | NONE | NONE | NONE | NONE | NONE | NONE |
| (SEQ ID NO: 297) | | | | | | | |
| NKPLFFADK(ac)LYK (SEQ ID NO: 298) | ANXA6 | − | − | + | 0.00 | 0.00 | −0.41 |
| ISK(ac)EEAMR (SEQ ID NO: 299) | RPL11 | NONE | NONE | NONE | NONE | NONE | NONE |
| AIFAGYK(ac)R (SEQ ID NO: 300) | RPL35A | NONE | NONE | NONE | NONE | NONE | NONE |
| AGLVDDFEK(ac)K (SEQ ID NO: 301) | ATP5H | NONE | NONE | NONE | NONE | NONE | NONE |
| LLEQYK(ac)EESK (SEQ ID NO: 302) | HNRNPU | NONE | NONE | NONE | NONE | NONE | NONE |
| VLVGK(ac)NFEDVAFDEK (SEQ ID NO: 303) | P4HB | NONE | NONE | NONE | NONE | NONE | NONE |
| LEMSYSK(ac)FK (SEQ ID NO: 304) | DPAGT1 | NONE | NONE | NONE | NONE | NONE | NONE |
| PAQGAK(ac)YR (SEQ ID NO: 305) | ANXA6 | NONE | NONE | NONE | NONE | NONE | NONE |
| IFGSNK(ac)WTTEQQQR (SEQ ID NO: 306) | ARL6IP1 | NONE | NONE | NONE | NONE | NONE | NONE |
| HVVQSISTQQEK(ac)ETIAK | ATP5F1 | NONE | NONE | NONE | NONE | NONE | NONE |
| (SEQ ID NO: 307) | | | | | | | |
| VPGK(ac)DTVTK (SEQ ID NO: 308) | HADHB | NONE | NONE | NONE | NONE | NONE | NONE |
| PEPAK(ac)SAPAPK(ac)K | HIST2H2BE | + | − | − | 0.56 | NA | 0.32 |
| (SEQ ID NO: 309) | | | | | | | |
| K(ac)STGGK(ac)APR (SEQ ID NO: 310) | HIST2H3D, | NONE | NONE | NONE | NONE | NONE | NONE |
| | HIST2H3A, | | | | | | |
| | HIST2H3C | | | | | | |
| GK(ac)GGK(ac)GLGK (SEQ ID NO: 311) | HIST2H4B, | NONE | NONE | NONE | NONE | NONE | NONE |
| | HIST1H4C, | | | | | | |
| | HIST1H4J, | | | | | | |
| | HIST1H4D, | | | | | | |
| | HIST1H4A, | | | | | | |
| | HIST2H4A, | | | | | | |
| | HIST1H4I, | | | | | | |
| | HIST1H4K, | | | | | | |
| | HIST1H4E, | | | | | | |
| | HIST1H4L, | | | | | | |
| | HIST1H4F, | | | | | | |
| | HIST1H4H, | | | | | | |
| | HIST4H4, | | | | | | |
| | HIST1H4B | | | | | | |
| GLGK(ac)GGAK(ac)R (SEQ ID NO: 312) | HIST2H4B, | − | − | + | 0.19 | 0.09 | 0.69 |
| | HIST1H4C, | | | | | | |
| | HIST1H4J, | | | | | | |
| | HIST1H4D, | | | | | | |
| | HIST1H4A, | | | | | | |
| | HIST2H4A, | | | | | | |
| | HIST1H4I, | | | | | | |
| | HIST1H4K, | | | | | | |
| | HIST1H4E, | | | | | | |
| | HIST1H4L, | | | | | | |
| | HIST1H4F, | | | | | | |
| | HIST1H4H, | | | | | | |
| | HIST4H4, | | | | | | |
| | HIST1H4B | | | | | | |
| GLGK(ac)GGAKR (SEQ ID NO: 313) | HIST2H4B, | NONE | NONE | NONE | NONE | NONE | NONE |
| | HIST1H4C, | | | | | | |
| | HIST1H4J, | | | | | | |
| | HIST1H4D, | | | | | | |
| | HIST1H4A, | | | | | | |
| | HIST2H4A, | | | | | | |
| | HIST1H4I, | | | | | | |
| | HIST1H4K, | | | | | | |
| | HIST1H4E, | | | | | | |
| | HIST1H4L, | | | | | | |
| | HIST1H4F, | | | | | | |
| | HIST1H4H, | | | | | | |
| | HIST4H4, | | | | | | |
| | HIST1H4B | | | | | | |
| EK(ac)NLLHVTDTGVGMTR | HSP90B1 | + | − | − | 0.40 | −0.28 | NA |
| (SEQ ID NO: 314) | | | | | | | |
| KSDYIK(ac)LYVR (SEQ ID NO: 315) | HSP90B1 | NONE | NONE | NONE | NONE | NONE | NONE |
| K(ac)SADTLWGIQK (SEQ ID NO: 316) | LDHA | − | − | + | 0.05 | 0.04 | 2.19 |
| IVADK(ac)DYSVTANSK (SEQ ID NO: 317) | LDHB | NONE | NONE | NONE | NONE | NONE | NONE |
| VVDSMDALDK(ac)VVQER (SEQ ID NO: 318) | MRPL47 | NONE | NONE | NONE | NONE | NONE | NONE |
| YSAK(ac)DYFFK (SEQ ID NO: 319) | NAPA | NONE | NONE | NONE | NONE | NONE | NONE |
| GIVLEK(ac)VGVEAK (SEQ ID NO: 320) | RPS23 | NONE | NONE | NONE | NONE | NONE | NONE |
| DLQQYQSQAK(ac)QLFR (SEQ ID NO: 321) | SEC22B | NONE | NONE | NONE | NONE | NONE | NONE |
| LLASK(ac)SLLNR (SEQ ID NO: 322) | SSR2 | NONE | NONE | NONE | NONE | NONE | NONE |
| TMSCSDK(ac)ILR (SEQ ID NO: 323) | ADAR | − | + | − | NA | −0.87 | NA |
| MVAAAK(ac)YAR (SEQ ID NO: 324) | ATP5C1 | + | + | − | −0.89 | −0.71 | NA |
| FEDPK(ac)FEVIEKPQA (SEQ ID NO: 325) | ATP5J | + | − | − | −0.61 | −0.02 | NA |
| VQDGLSDIAEK(ac)FLK (SEQ ID NO: 326) | ATRX | − | − | + | NA | NA | 0.89 |
| LNK(ac)PFLFDTK (SEQ ID NO: 327) | CANX | − | + | − | NA | 0.43 | NA |
| AAEEVEAK(ac)FK (SEQ ID NO: 328) | CHCHD3 | − | + | − | NA | −0.53 | −0.08 |
| SLLK(ac)PFCAALLK (SEQ ID NO: 329) | CKAP5 | − | − | + | NA | NA | 0.48 |
| SMSTEGLMK(ac)FVDSK (SEQ ID NO: 330) | CS | − | + | − | NA | 0.76 | NA |
| YNIEK(ac)DIAAYIK (SEQ ID NO: 331) | DYNLL2 | − | + | − | NA | −0.50 | 0.03 |
| YLAEK(ac)YEWDVAEAR (SEQ ID NO: 332) | EEF2 | + | + | − | −0.52 | −0.40 | −0.11 |
| FK(ac)ILDAVVAQEPLHR (SEQ ID NO: 333) | EFTUD2 | − | − | + | NA | NA | −0.99 |
| LEAMCFDGVK(ac)R (SEQ ID NO: 334) | EIF1AXP1 | + | − | − | −0.62 | −0.17 | NA |
| LMCK(ac)PIFSK (SEQ ID NO: 335) | EIF2S3 | + | + | + | 1.18 | 0.41 | 0.38 |
| SNPSEVVEFTTCPDK(ac)PGIPVKPSVK | FNDC3A | − | + | − | NA | 0.40 | NA |
| (SEQ ID NO: 336) | | | | | | | |
| EIAQEFK(ac)TDLR (SEQ ID NO: 337) | HIST2H3PS2 | − | − | + | NA | NA | −0.49 |
| APQCLGK(ac)FIEIAAR (SEQ ID NO: 338) | HNRNPU | + | + | + | 0.99 | 0.77 | 0.47 |
| MMVAGFK(ac)K (SEQ ID NO: 339) | HNRNPU | − | − | + | NA | NA | −0.40 |
| FEYK(ac)YSFK (SEQ ID NO: 340) | LMAN1 | − | + | − | NA | −0.52 | NA |
| IINEVSK(ac)PLAHHIPVEK | MANF | − | + | − | NA | −0.58 | NA |
| (SEQ ID NO: 341) | | | | | | | |
| FLNK(ac)LAEER (SEQ ID NO: 342) | MATR3, | − | + | − | NA | −0.44 | NA |
| | SNHG4 | | | | | | |
| VHLSQK(ac)YK (SEQ ID NO: 343) | MATR3, | − | − | + | NA | NA | 0.40 |
| | SNHG4 | | | | | | |
| INK(ac)ALDK (SEQ ID NO: 344) | MYH9 | NONE | NONE | NONE | NONE | NONE | NONE |
| FCLSK(ac)PGVYK (SEQ ID NO: 345) | NOMO3, | NONE | NONE | NONE | NONE | NONE | NONE |
| | NOMO1 | | | | | | |
| AQNDLIWNIK(ac)DELKK (SEQ ID NO: 346) | PARP1 | − | − | + | NA | NA | 0.83 |
| FPDENFK(ac)LK (SEQ ID NO: 347) | PPIC | − | − | + | NA | NA | −1.15 |
| TNVPVK(ac)LFAR (SEQ ID NO: 348) | RALY | − | − | + | NA | NA | 0.39 |
| GYAYVEFENPDEAEK(ac)ALK | RNPS1 | − | + | + | NA | −0.38 | −0.54 |
| (SEQ ID NO: 349) | | | | | | | |
| VTNLLMLK(ac)GK (SEQ ID NO: 350) | ROD1 | − | − | + | NA | NA | 0.83 |
| KIEISQHAK(ac)YTCSFCGK | RPL37A | − | + | − | 0.24 | 1.97 | 0.32 |
| (SEQ ID NO: 351) | | | | | | | |
| DTYIENEK(ac)LISGK (SEQ ID NO: 352) | RPN1 | − | + | − | NA | 0.77 | NA |
| KDVHMPK(ac)HPELADK (SEQ ID NO: 353) | RPS10 | − | + | − | NA | 0.56 | NA |
| TFEK(ac)SMMNLQTK (SEQ ID NO: 354) | TOP1 | − | − | + | NA | NA | 0.46 |
| KQELLEALTK(ac)HFQD (SEQ ID NO: 355) | XRCC6 | − | + | − | NA | −1.25 | −0.23 |
| NFK(ac)SISK (SEQ ID NO: 356) | ACADVL | − | + | − | 0.29 | 0.59 | NA |
| TNFSNEK(ac)TISK (SEQ ID NO: 357) | ACYP2 | − | + | − | NA | 0.84 | NA |
| K(ac)YGKSLYYYIQQDTK (SEQ ID NO: 358) | ANXA2P2 | − | + | − | NA | 0.66 | NA |
| RPEILK(ac)QEIK (SEQ ID NO: 359) | AP2B1 | − | + | − | NA | 0.57 | NA |
| ELQEMDKDDESLIK(ac)YK | ARHGDIB | − | + | − | NA | 0.41 | NA |
| (SEQ ID NO: 360) | | | | | | | |
| NAYVWILK(ac)GR (SEQ ID NO: 361) | ARPC1B, | NONE | NONE | NONE | NONE | NONE | NONE |
| | ARPC1A | | | | | | |
| VFDPQNDK(ac)PSKWWTCFVK | ARPC3 | − | + | − | 0.32 | 0.45 | NA |
| (SEQ ID NO: 362) | | | | | | | |
| VLISFK(ac)ANDIEK (SEQ ID NO: 363) | ARPC5 | − | + | − | NA | 0.42 | NA |
| LAALPENPPAIDWAYYK(ac)ANVAK | ATP5H | − | + | − | NA | 0.48 | NA |
| (SEQ ID NO: 364) | | | | | | | |
| ELDPIQK(ac)LFVDK (SEQ ID NO: 365) | ATP5J | − | + | − | NA | −0.48 | NA |
| GHCPPAPAK(ac)PMHPENK(ac)LTNHGK | BCL9L | + | − | − | 0.63 | NA | NA |
| (SEQ ID NO: 366) | | | | | | | |
| LVGELK(ac)LDR (SEQ ID NO: 367) | BPI | − | + | − | NA | −0.52 | NA |
| SNYK(ac)MMFVK (SEQ ID NO: 368) | C11orf58 | + | − | − | −0.45 | NA | NA |
| K(ac)HDSGAADLER (SEQ ID NO: 369) | C15orf63 | + | + | − | 2.73 | 2.66 | NA |
| AELSK(ac)LVIVAK (SEQ ID NO: 370) | C19orf10 | − | + | − | NA | 1.55 | NA |
| LHSNYYK(ac)HK (SEQ ID NO: 371) | C6orf125 | + | − | − | 0.52 | NA | NA |
| GQTLVVQFTVK(ac)HEQNIDCGGGYVK | CALR | − | + | − | NA | −0.43 | NA |
| (SEQ ID NO: 372) | | | | | | | |
| K(ac)VCGDSDKGFVVINQK | CCT6A | + | − | − | 0.67 | NA | NA |
| (SEQ ID NO: 373) | | | | | | | |
| (ac)MEDYTK(ac)IEK (SEQ ID NO: 374) | CDK1 | + | + | − | −0.51 | −0.40 | NA |
| LK(ac)ADVFK (SEQ ID NO: 375) | CHD4 | − | − | + | NA | NA | −0.45 |
| (ac)SNMEK(ac)HLFNLK (SEQ ID NO: 376) | CHMP1B | NONE | NONE | NONE | NONE | NONE | NONE |
| GK(ac)YMLVR (SEQ ID NO: 377) | CHST1 | + | − | − | 3.73 | NA | NA |
| FLDGNEMTLADCNLLPK(ac)LHIVK | CLIC4 | − | + | − | NA | 0.51 | NA |
| (SEQ ID NO: 378) | | | | | | | |
| (ac)SSNECFK(ac)CGR (SEQ ID NO: 379) | CNBP | + | − | − | −1.09 | NA | NA |
| NLK(ac)NEITK (SEQ ID NO: 380) | COPA | − | + | − | NA | 0.40 | NA |
| DHPLPEVAHVK(ac)HLSASQK | COX4I1 | − | + | − | NA | −0.63 | NA |
| (SEQ ID NO: 381) | | | | | | | |
| NLPFSVENK(ac)WSLLAK (SEQ ID NO: 382) | COX7C | − | + | − | NA | −0.87 | NA |
| GLESTTLADK(ac)DGEIYCK | CSRP1 | + | − | − | −0.67 | NA | NA |
| (SEQ ID NO: 383) | | | | | | | |
| HTENVAK(ac)FHCPHCDTVIAR | CTCF | − | − | + | NA | NA | −0.66 |
| (SEQ ID NO: 384) | | | | | | | |
| LHYK(ac)HESWLLHR (SEQ ID NO: 385) | DCK | − | + | − | NA | −0.43 | NA |
| QLNDVK(ac)TTVVYPATEK | DCPS | NONE | NONE | NONE | NONE | NONE | NONE |
| (SEQ ID NO: 386) | | | | | | | |
| SHSAHFFEFLTK(ac)ELALGQDR | DDT | − | + | − | NA | −0.44 | NA |
| (SEQ ID NO: 387) | | | | | | | |
| KDFIK(ac)TTVK (SEQ ID NO: 388) | DEK | − | − | + | NA | NA | 0.38 |
| LCMLYHPDK(ac)HRDPELK | DNAJC11 | − | + | − | NA | −0.69 | NA |
| (SEQ ID NO: 389) | | | | | | | |
| LCK(ac)YIYAK (SEQ ID NO: 390) | EIF3C, | − | + | − | NA | 0.45 | NA |
| | EIF3CL | | | | | | |
| KPEENPASK(ac)FSSASK (SEQ ID NO: 391) | EIF4B | + | − | − | 0.53 | NA | NA |
| EENTSNESTDVTK(ac)GDSK(ac)NAK | EP300 | + | + | − | 0.53 | 0.39 | NA |
| (SEQ ID NO: 392) | | | | | | | |
| GLAPQNK(ac)PELQK (SEQ ID NO: 393) | FAM107B | + | − | − | 0.69 | NA | NA |
| VYISK(ac)CYR (SEQ ID NO: 394) | FAM82A2 | − | + | − | NA | −0.85 | NA |
| SFYYK(ac)LR (SEQ ID NO: 395) | FASN | − | − | + | NA | NA | −0.73 |
| AVSMDNSNK(ac)YTK (SEQ ID NO: 396) | FOXO3 | + | − | − | 0.45 | NA | NA |
| FAFK(ac)EVEVQAK (SEQ ID NO: 397) | FTSJ3 | − | − | + | NA | NA | 0.74 |
| LVQTAELTK(ac)VFEIR (SEQ ID NO: 398) | GMFG | − | + | − | NA | −0.80 | NA |
| LWNTLGVCK(ac)YTVQDESHSEWVSCVR | GNB2L1 | − | + | + | NA | 0.60 | 0.47 |
| (SEQ ID NO: 399) | | | | | | | |
| EFTK(ac)LEEVLTNK (SEQ ID NO: 400) | GSTO1 | + | − | − | −0.60 | NA | NA |
| VIIVVK(ac)DGPGFYTTR (SEQ ID NO: 401) | HADHA | − | + | − | NA | −0.70 | NA |
| AK(ac)FESMAEEK (SEQ ID NO: 402) | HCLS1 | + | + | − | 1.21 | 0.41 | NA |
| ATAEEMTK(ac)YHSDEYIK | HDAC2 | NONE | NONE | NONE | NONE | NONE | NONE |
| (SEQ ID NO: 403) | | | | | | | |
| ESESVDK(ac)VMDQK (SEQ ID NO: 404) | HNRNPD | − | − | + | NA | NA | −0.53 |
| VFITDDFHDMMPK(ac)YLNFVK | HSP90B1 | − | + | − | NA | 0.77 | NA |
| (SEQ ID NO: 405) | | | | | | | |
| IQEIIEQLDVTTSEYEK(ac)EKLNER | HSPD1 | + | − | − | 0.40 | NA | NA |
| (SEQ ID NO: 406) | | | | | | | |
| TFYQMYDK(ac)GLVYR (SEQ ID NO: 407) | IARS2 | + | − | − | −0.56 | NA | NA |
| NVTAIQGPGGK(ac)WMIPSEAK | IDH3A | + | − | − | −0.44 | NA | NA |
| (SEQ ID NO: 408) | | | | | | | |
| KSEDTISK(ac)MNDFMR (SEQ ID NO: 409) | IFI16 | − | − | + | NA | NA | −0.57 |
| SQSCETK(ac)LLDEK (SEQ ID NO: 410) | 1L16 | + | − | − | −0.53 | NA | NA |
| KK(ac)FLCVTK (SEQ ID NO: 411) | KBTBD8 | + | − | − | 2.55 | NA | NA |
| IQFK(ac)QDDGTGPEK (SEQ ID NO: 412) | KHSRP | − | + | − | NA | 0.61 | NA |
| K(ac)GPSVQKR (SEQ ID NO: 413) | LIN28B | + | + | − | 0.50 | 0.47 | NA |
| LK(ac)AEKYR (SEQ ID NO: 414) | LRRIQ3 | − | + | − | NA | −1.77 | NA |
| FQAK(ac)LEHEYIQNFK (SEQ ID NO: 415) | MAPRE1 | − | + | − | NA | −1.51 | NA |
| NVQQTVSAK(ac)GPPEK(ac)R | MED6 | + | − | − | 0.49 | NA | NA |
| (SEQ ID NO: 416) | | | | | | | |
| YK(ac)VEYPIMYSTDPENGHIFNCIQR | MGST3 | − | + | − | NA | −0.59 | NA |
| (SEQ ID NO: 417) | | | | | | | |
| TPSVGK(ac)AMDTPKPAGGDEK | MKI67 | − | − | + | NA | NA | −0.86 |
| (SEQ ID NO: 418) | | | | | | | |
| IIWHK(ac)FK (SEQ ID NO: 419) | MRPL47 | − | + | − | NA | −0.73 | NA |
| FK(ac)ECLDK (SEQ ID NO: 420) | NAA15 | − | + | − | NA | 0.60 | NA |
| (ac)AQLGK(ac)LLK (SEQ ID NO: 421) | NAE1 | NONE | NONE | NONE | NONE | NONE | NONE |
| SELEFK(ac)FNR (SEQ ID NO: 422) | NANS | + | − | − | 0.53 | NA | NA |
| YTEQITNEK(ac)LAMVK (SEQ ID NO: 423) | NDUFA5 | − | + | − | NA | −0.40 | NA |
| YHVSEK(ac)PYGIVEK (SEQ ID NO: 424) | NDUFB6 | − | + | − | NA | −1.08 | NA |
| CHAFEK(ac)EWIECAHGIGYTR | NDUFS5 | − | + | − | NA | −0.63 | NA |
| (SEQ ID NO: 425) | | | | | | | |
| DVTK(ac)AMDEK (SEQ ID NO: 426) | PFKM | − | − | + | NA | NA | 0.53 |
| EGVHGGLINK(ac)K (SEQ ID NO: 427) | PFN1 | + | − | − | 0.58 | NA | NA |
| TVSK(ac)VDDFLANEAK (SEQ ID NO: 428) | PGD | − | + | − | NA | 0.74 | NA |
| ITLPVDFVTADK(ac)FDENAK | PGK1 | − | + | − | NA | 0.60 | NA |
| (SEQ ID NO: 429) | | | | | | | |
| NVTGHYISPFHDIPLK(ac)VNSK | PPA2 | − | − | + | NA | NA | 0.91 |
| (SEQ ID NO: 430) | | | | | | | |
| FEDENFHYK(ac)HDR (SEQ ID NO: 431) | PPID | − | + | − | NA | 0.46 | NA |
| HSPEDPEK(ac)YSCFALFVK | PRKDC | − | − | + | NA | NA | −0.41 |
| (SEQ ID NO: 432) | | | | | | | |
| MLK(ac)QMLFR (SEQ ID NO: 433) | PSMB7 | − | + | − | NA | −0.46 | NA |
| IMK(ac)SEVLR (SEQ ID NO: 434) | PSMC3 | NONE | NONE | NONE | NONE | NONE | NONE |
| TTHLIAK(ac)EEMIHNLQ (SEQ ID NO: 435) | PSMD12 | + | − | − | −0.38 | NA | NA |
| VAHLALK(ac)HR (SEQ ID NO: 436) | PSMD4 | − | + | − | NA | −0.57 | NA |
| (ac)SDAAVDTSSEITTK(ac)DLK | PTMA, | + | + | − | 0.57 | 1.03 | NA |
| (SEQ ID NO: 437) | MIR1244-3, | | | | | | |
| | MIR1244-2, | | | | | | |
| | MIR1244-1 | | | | | | |
| HMVMQK(ac)LLR (SEQ ID NO: 438) | PUF60 | + | + | + | 0.71 | 0.89 | 0.54 |
| AFQLK(ac)SR (SEQ ID NO: 439) | RAVER1 | − | + | − | NA | −0.89 | NA |
| IMQK(ac)YGFR (SEQ ID NO: 440) | RBM17 | − | − | + | NA | NA | −0.50 |
| GNLYSFGCPEYGQLGHNSDGK(ac)FIAR | RCC2 | − | + | − | NA | 0.75 | NA |
| (SEQ ID NO: 441) | | | | | | | |
| WTPEVK(ac)HFCPNVPIILVGNK | RHOC | − | + | − | NA | −0.39 | NA |
| (SEQ ID NO: 442) | | | | | | | |
| AYHLQK(ac)STCGK (SEQ ID NO: 443) | RPL37 | NONE | NONE | NONE | NONE | NONE | NONE |
| NVYK(ac)FELDTSER (SEQ ID NO: 444) | RPN2 | − | + | − | NA | 0.54 | NA |
| VETFSGVYK(ac)K (SEQ ID NO: 445) | RPS7 | − | + | − | NA | −0.78 | NA |
| MLEK(ac)DVCEK (SEQ ID NO: 446) | RUFY3 | + | − | − | 0.62 | NA | NA |
| VEWAK(ac)FQER (SEQ ID NO: 447) | SF3A1 | − | − | + | NA | NA | −0.55 |
| LNPK(ac)TGLIDYNQLALTAR | SHMT2 | + | − | − | −0.41 | NA | NA |
| (SEQ ID NO: 448) | | | | | | | |
| GNTPK(ac)YGLIFHSTFIGR | SNORD110, | − | − | + | NA | NA | −0.41 |
| (SEQ ID NO: 449) | SNORD86, | | | | | | |
| | NOP56 | | | | | | |
| HHAAYVNNLNVTEEK(ac)YQEALAK | SOD2 | + | − | − | −0.67 | NA | NA |
| (SEQ ID NO: 450) | | | | | | | |
| TITCSK(ac)LAYYYQR (SEQ ID NO: 451) | SRP54 | − | − | + | NA | NA | 0.40 |
| DK(ac)WLCPLSGK (SEQ ID NO: 452) | SRRT | − | − | + | NA | NA | 0.42 |
| IIEDQQESLNK(ac)WK (SEQ ID NO: 453) | SSB | + | − | − | 0.57 | NA | NA |
| IDYGEYMDK(ac)NNVR (SEQ ID NO: 454) | SSBP1 | − | + | − | NA | −0.52 | NA |
| GEDFPANNIVK(ac)FLVGFTNK | SSR1 | − | + | − | NA | −0.88 | NA |
| (SEQ ID NO: 455) | | | | | | | |
| DK(ac)LAQQQAAAAAAAAAAASQQGSAK | TBL1XR1 | + | + | − | 0.89 | 0.73 | NA |
| (SEQ ID NO: 456) | | | | | | | |
| IAVEAQNK(ac)YER (SEQ ID NO: 457) | TPR | − | + | − | NA | 0.62 | NA |
| SQNTK(ac)ISTQLDFASK (SEQ ID NO: 458) | TPR | − | + | − | NA | −0.45 | NA |
| GK(ac)LEEQRPER (SEQ ID NO: 459) | TPT1 | − | + | − | NA | −0.42 | NA |
| QPAENVNQYLTDPK(ac)FVER | UBA1 | + | − | − | 0.44 | NA | NA |
| (SEQ ID NO: 460) | | | | | | | |
| YPPPYK(ac)HFWTAES (SEQ ID NO: 461) | UBAP2L | NONE | NONE | NONE | NONE | NONE | NONE |
| IYHPNVDK(ac)LGR (SEQ ID NO: 462) | UBE2N | − | + | − | NA | −0.70 | NA |
| ACK(ac)LAIR (SEQ ID NO: 463) | VCP | − | + | − | NA | −0.58 | NA |
| (ac)AVPPTYADLGK(ac)SAR | VDAC1 | − | + | − | NA | −0.78 | NA |
| (SEQ ID NO: 464) | | | | | | | |
| GYGFGLIK(ac)LDLK (SEQ ID NO: 465) | VDAC1 | − | + | − | NA | −0.57 | NA |
| LTLSALLDGK(ac)NVNAGGHK | VDAC1 | − | + | − | NA | −0.66 | NA |
| (SEQ ID NO: 466) | | | | | | | |
| DVFNK(ac)GYGFGMVK (SEQ ID NO: 467) | VDAC3 | − | + | − | NA | −0.73 | NA |
| FGIAAK(ac)YMLDCR (SEQ ID NO: 468) | VDAC3 | − | + | + | NA | −0.89 | 0.55 |
| LTLSALIDGK(ac)NFSAGGHK | VDAC3 | − | + | − | NA | −0.71 | NA |
| (SEQ ID NO: 469) | | | | | | | |
| SGK(ac)FMNPTDQAR (SEQ ID NO: 470) | WBP11 | + | + | − | −0.45 | −0.68 | NA |
| QLGSILK(ac)TNVR (SEQ ID NO: 471) | XPO1 | + | − | − | 0.93 | NA | NA |
| AIVEK(ac)LR (SEQ ID NO: 472) | XRCC6 | − | − | + | NA | NA | 1.89 |
| CPICEK(ac)VIQGAGK (SEQ ID NO: 473) | ZBTB7A | + | − | − | −0.44 | NA | NA |
| SIDFPLTK(ac)VYVVEGSK | ZMPSTE24 | − | + | − | NA | 0.61 | NA |
| (SEQ ID NO: 474) | | | | | | | |
| IIEK(ac)DVMEGVTVDDHMMK | ZNF326 | − | − | + | NA | NA | 0.40 |
| (SEQ ID NO: 475) | | | | | | | |
| LVSEK(ac)LASYQAAR (SEQ ID NO: 476) | MYC | + | + | − | −0.55 | −0.58 | −0.26 |
| SFDK(ac)NLYR (SEQ ID NO: 477) | MCM4 | − | + | − | NA | −0.57 | 0.26 |
| LVDTGDK(ac)FR (SEQ ID NO: 478) | SPTBN1 | − | + | − | −0.26 | −0.42 | −0.30 |
| FGIAAK(ac)YQLDPTASISAK | VDAC2 | − | + | − | NA | −0.74 | 0.35 |
| (SEQ ID NO: 479) | | | | | | | |
| IK(ac)EKYIDQEELNK (SEQ ID NO: 480) | HSP90AA1 | + | − | − | 0.45 | NA | NA |
| SYAEELAK(ac)HGMK (SEQ ID NO: 481) | HSD17B12 | + | + | − | 0.41 | 0.73 | NA |
| EHLLK(ac)YEYQPFAGK (SEQ ID NO: 482) | WDR1 | − | + | − | 0.33 | 0.45 | −0.13 |
| GVHPK(ac)FPEGGK (SEQ ID NO: 483) | CYB5R1 | − | + | − | NA | −0.48 | NA |
| LAK(ac)LSDGVAVLK (SEQ ID NO: 484) | HSPD1 | + | − | − | −0.57 | 0.00 | NA |
| LTLDK(ac)LDVK (SEQ ID NO: 485) | PGK1 | − | + | − | NA | −0.39 | NA |
| DVTLQK(ac)QCVPFR (SEQ ID NO: 486) | RPL17 | − | − | + | NA | NA | −0.38 |
| FVATGHGK(ac)YEK (SEQ ID NO: 487) | LOC100133284, | + | + | − | 1.24 | 1.02 | NA |
| | LSP1 | | | | | | |
| VAILK(ac)ANLR (SEQ ID NO: 488) | VCP | + | + | − | 2.84 | 1.09 | NA |
| IVQK(ac)HPHTGDTKEEK (SEQ ID NO: 489) | DAP | + | + | − | 0.91 | 0.80 | NA |
| GSK(ac)K(ac)AVIK (SEQ ID NO: 490) | HIST2H2BE | NONE | NONE | NONE | NONE | NONE | NONE |
| PKPYDPPGEK(ac)MVAAK (SEQ ID NO: 491) | ATF4 | NONE | NONE | NONE | NONE | NONE | NONE |
| RPPQTIK(ac)SVR (SEQ ID NO: 492) | C15orf58 | − | − | + | NA | NA | −5.00 |
| STPAITLESPDIK(ac)YPLR | CYB5R3 | NONE | NONE | NONE | NONE | NONE | NONE |
| (SEQ ID NO: 493) | | | | | | | |
| HGMK(ac)VVLISR (SEQ ID NO: 494) | HSD17B12 | NONE | NONE | NONE | NONE | NONE | NONE |
| LALQK(ac)QLTK (SEQ ID NO: 495) | ZNF385C | NONE | NONE | NONE | NONE | NONE | NONE |
| NK(ac)YGDAFIR (SEQ ID NO: 496) | LSM6 | NONE | NONE | NONE | NONE | NONE | NONE |
| AK(ac)HDELTYF (SEQ ID NO: 497) | CSTB | NONE | NONE | NONE | NONE | NONE | NONE |
| NK(ac)WFFQK (SEQ ID NO: 498) | RPL27 | NONE | NONE | NONE | NONE | NONE | NONE |
| LMK(ac)YGDSLYR (SEQ ID NO: 499) | GTPBP4 | NONE | NONE | NONE | NONE | NONE | NONE |
| LPSSTK(ac)SGWPR (SEQ ID NO: 500) | PHF15 | NONE | NONE | NONE | NONE | NONE | NONE |
| TLAMDVMK(ac)PR (SEQ ID NO: 501) | CLCN5 | NONE | NONE | NONE | NONE | NONE | NONE |
| SAQEAYK(ac)SYIR (SEQ ID NO: 502) | DDX18 | NONE | NONE | NONE | NONE | NONE | NONE |
| AEK(ac)DEPGAWEETFK (SEQ ID NO: 503) | GANAB | NONE | NONE | NONE | NONE | NONE | NONE |
| FSPDDK(ac)YSR (SEQ ID NO: 504) | NOP10 | NONE | NONE | NONE | NONE | NONE | NONE |
| LAAIVAK(ac)QVLLGR (SEQ ID NO: 505) | SNORD32A, | NONE | NONE | NONE | NONE | NONE | NONE |
| | RPL13A, | | | | | | |
| | SNORD33, | | | | | | |
| | SNORD34 | | | | | | |
| IDK(ac)PILK (SEQ ID NO: 506) | RPL8 | NONE | NONE | NONE | NONE | NONE | NONE |
| LVPLK(ac)ETIK (SEQ ID NO: 507) | ATP5B | NONE | NONE | NONE | NONE | NONE | NONE |
| (ac)AEPVSPLK(ac)HFVLAK | MFN1 | NONE | NONE | NONE | NONE | NONE | NONE |
| (SEQ ID NO: 508) | | | | | | | |
| EEAIK(ac)FSEEQR (SEQ ID NO: 509) | XRCC5 | NONE | NONE | NONE | NONE | NONE | NONE |
| K(ac)EELTLEGIR (SEQ ID NO: 510) | EIF4A1 | NONE | NONE | NONE | NONE | NONE | NONE |
| TLFVK(ac)GLSEDTTEETLK | NCL | NONE | NONE | NONE | NONE | NONE | NONE |
| (SEQ ID NO: 511) | | | | | | | |
| LIAGNK(ac)PVSFLTAQQLQQLQQQGQATQVR | NFRKB | NONE | NONE | NONE | NONE | NONE | NONE |
| (SEQ ID NO: 512) | | | | | | | |
| ALK(ac)YLVMDEADR (SEQ ID NO: 513) | DDX47 | NONE | NONE | NONE | NONE | NONE | NONE |
| LLLFK(ac)TFSR (SEQ ID NO: 514) | HNRNPUL2 | NONE | NONE | NONE | NONE | NONE | NONE |
| VLGTVK(ac)WFNVR (SEQ ID NO: 515) | CSDA | NONE | NONE | NONE | NONE | NONE | NONE |
| TLK(ac)ALEYVFK (SEQ ID NO: 516) | DOCK2 | NONE | NONE | NONE | NONE | NONE | NONE |
| LMEK(ac)EINGSK (SEQ ID NO: 517) | ATL3 | NONE | NONE | NONE | NONE | NONE | NONE |
| AVDCLLDSK(ac)WAK (SEQ ID NO: 518) | SEC62 | NONE | NONE | NONE | NONE | NONE | NONE |
| GHK(ac)FHHTIGGSR (SEQ ID NO: 519) | RPL15 | NONE | NONE | NONE | NONE | NONE | NONE |
| VYVLK(ac)FK (SEQ ID NO: 520) | ADRM1 | NONE | NONE | NONE | NONE | NONE | NONE |
| LSFISVGNK(ac)FK (SEQ ID NO: 521) | MYO6 | NONE | NONE | NONE | NONE | NONE | NONE |
| VITEEEK(ac)NFK (SEQ ID NO: 522) | RPL13 | NONE | NONE | NONE | NONE | NONE | NONE |
| TLVLSDK(ac)HSPQK (SEQ ID NO: 523) | MLL3 | NONE | NONE | NONE | NONE | NONE | NONE |
| SQFLK(ac)YIEK (SEQ ID NO: 524) | MCM2 | NONE | NONE | NONE | NONE | NONE | NONE |
| SEIINSK(ac)NFDR (SEQ ID NO: 525) | TMEM43 | NONE | NONE | NONE | NONE | NONE | NONE |
| MQK(ac)EITALAPSTMK (SEQ ID NO: 526) | ACTA2 | NONE | NONE | NONE | NONE | NONE | NONE |
| ETGYTYILPK(ac)NVLK (SEQ ID NO: 527) | PRPF8 | NONE | NONE | NONE | NONE | NONE | NONE |
| LLNK(ac)HGIK (SEQ ID NO: 528) | RSL1D1 | NONE | NONE | NONE | NONE | NONE | NONE |
| K(ac)VLFTCFK (SEQ ID NO: 529) | TOP2B | NONE | NONE | NONE | NONE | NONE | NONE |
| DMCLEK(ac)DTLGLFLR (SEQ ID NO: 530) | SNRNP200 | NONE | NONE | NONE | NONE | NONE | NONE |
| IAAYK(ac)SILQER (SEQ ID NO: 531) | XRCC5 | NONE | NONE | NONE | NONE | NONE | NONE |
| YAVLYQPLFDK(ac)R (SEQ ID NO: 532) | NAP1L1 | NONE | NONE | NONE | NONE | NONE | NONE |
| SHLAK(ac)EGLYQYK (SEQ ID NO: 533) | EIF3A | NONE | NONE | NONE | NONE | NONE | NONE |
| YKDAIHFYNK(ac)SLAEHR | STIP1 | NONE | NONE | NONE | NONE | NONE | NONE |
| (SEQ ID NO: 534) | | | | | | | |
| YIGK(ac)TMDYR (SEQ ID NO: 535) | MRPL24 | NONE | NONE | NONE | NONE | NONE | NONE |
| GLCEK(ac)PLASAAAK (SEQ ID NO: 536) | TNPO3 | NONE | NONE | NONE | NONE | NONE | NONE |
| GK(ac)FGNVYLAR (SEQ ID NO: 537) | AURKC | NONE | NONE | NONE | NONE | NONE | NONE |
| LWSK(ac)AIFAGYK (SEQ ID NO: 538) | RPL35A | NONE | NONE | NONE | NONE | NONE | NONE |
| LQVLDPVPK(ac)PVIK (SEQ ID NO: 539) | CD48 | NONE | NONE | NONE | NONE | NONE | NONE |
| ELK(ac)EIQYGIR (SEQ ID NO: 540) | BCAT2 | NONE | NONE | NONE | NONE | NONE | NONE |
| GYPTIK(ac)FFR (SEQ ID NO: 541) | P4HB | NONE | NONE | NONE | NONE | NONE | NONE |
| IHGVGFK(ac)K (SEQ ID NO: 542) | RPL31 | NONE | NONE | NONE | NONE | NONE | NONE |
| ISGASEK(ac)DIVHSGLAYTMER | GLUD1 | NONE | NONE | NONE | NONE | NONE | NONE |
| (SEQ ID NO: 543) | | | | | | | |
| PK(ac)PLLFK (SEQ ID NO: 544) | UGGT1 | NONE | NONE | NONE | NONE | NONE | NONE |
| FNNFLK(ac)ALQEK (SEQ ID NO: 545) | XRCC5 | NONE | NONE | NONE | NONE | NONE | NONE |
| AVDLIQK(ac)HK (SEQ ID NO: 546) | FEN1 | NONE | NONE | NONE | NONE | NONE | NONE |
| AEEILEK(ac)GLK (SEQ ID NO: 547) | RPL11 | NONE | NONE | NONE | NONE | NONE | NONE |
| LFDYFPK(ac)PYPNSEAAR | CYC1 | NONE | NONE | NONE | NONE | NONE | NONE |
| (SEQ ID NO: 548) | | | | | | | |
| AHLQK(ac)LMSDK (SEQ ID NO: 549) | GARS | NONE | NONE | NONE | NONE | NONE | NONE |
| TFK(ac)GVVDVVMK (SEQ ID NO: 550) | GFM2 | NONE | NONE | NONE | NONE | NONE | NONE |
| AAWEHMK(ac)K (SEQ ID NO: 551) | HSD17B4 | NONE | NONE | NONE | NONE | NONE | NONE |
| STVAQLVK(ac)R (SEQ ID NO: 552) | ATP5A1 | NONE | NONE | NONE | NONE | NONE | NONE |
| YNIEK(ac)DIAAHIK (SEQ ID NO: 553) | DYNLL1 | NONE | NONE | NONE | NONE | NONE | NONE |
| HFAFK(ac)MASGAANVVGPK | FAM3C | NONE | NONE | NONE | NONE | NONE | NONE |
| (SEQ ID NO: 554) | | | | | | | |
| TDFDKNK(ac)IWYEHR (SEQ ID NO: 555) | IDH2 | NONE | NONE | NONE | NONE | NONE | NONE |
| HGYIK(ac)GIVK (SEQ ID NO: 556) | RPL8 | NONE | NONE | NONE | NONE | NONE | NONE |
| FAK(ac)PVYPGQTLQTEMWK | HSD17B4 | NONE | NONE | NONE | NONE | NONE | NONE |
| (SEQ ID NO: 557) | | | | | | | |
| NCSSFLIK(ac)R (SEQ ID NO: 558) | RPL28 | − | − | + | 0.05 | −0.15 | −0.40 |
| NK(ac)IAGYVTHLMK (SEQ ID NO: 559) | RPS17, | NONE | NONE | NONE | NONE | NONE | NONE |
| | RPS17L | | | | | | |
| AVTK(ac)AQK(ac)K (SEQ ID NO: 560) | HIST2H2BE | NONE | NONE | NONE | NONE | NONE | NONE |
| VK(ac)ILFNK (SEQ ID NO: 561) | PTBP1 | NONE | NONE | NONE | NONE | NONE | NONE |
| VK(ac)FIHDOTSPNPK (SEQ ID NO: 562) | SLC25A1 | NONE | NONE | NONE | NONE | NONE | NONE |
| YFDEK(ac)IAK (SEQ ID NO: 563) | BDH1 | NONE | NONE | NONE | NONE | NONE | NONE |
| LSLLSK(ac)FR (SEQ ID NO: 564) | HADHB | NONE | NONE | NONE | NONE | NONE | NONE |
| VK(ac)NELFK (SEQ ID NO: 565) | HADH | NONE | NONE | NONE | NONE | NONE | NONE |
| EVGKDVSDEK(ac)LR (SEQ ID NO: 566) | CS | NONE | NONE | NONE | NONE | NONE | NONE |
| SFLLK(ac)DSETSQR (SEQ ID NO: 567) | SHMT2 | NONE | NONE | NONE | NONE | NONE | NONE |
| VFEK(ac)YGR (SEQ ID NO: 568) | SRSF2 | NONE | NONE | NONE | NONE | NONE | NONE |
| NSNPALNDNLEK(ac)GLLK | CLIC1 | NONE | NONE | NONE | NONE | NONE | NONE |
| (SEQ ID NO: 569) | | | | | | | |
| LAAFGQLHK(ac)VLGMDPLPSK | ILF3 | NONE | NONE | NONE | NONE | NONE | NONE |
| (SEQ ID NO: 570) | | | | | | | |
| DVSYQGGK(ac)SIK (SEQ ID NO: 571) | MLL3 | NONE | NONE | NONE | NONE | NONE | NONE |
| SIDLK(ac)DK (SEQ ID NO: 572) | HSPD1 | NONE | NONE | NONE | NONE | NONE | NONE |
| MTDK(ac)CFR (SEQ ID NO: 573) | TIMM13 | NONE | NONE | NONE | NONE | NONE | NONE |
| HAHGDQYK(ac)ATDFVADR | IDH2 | NONE | NONE | NONE | NONE | NONE | NONE |
| (SEQ ID NO: 574) | | | | | | | |
| (ac)ACGLVASNLNLK(ac)PGECLR | LGALS1 | NONE | NONE | NONE | NONE | NONE | NONE |
| (SEQ ID NO: 575) | | | | | | | |
| HVVFGK(ac)VK (SEQ ID NO: 576) | PPIAP26 | NONE | NONE | NONE | NONE | NONE | NONE |
| AVLIDK(ac)DQSPK (SEQ ID NO: 577) | HIBCH | − | + | − | 0.00 | 0.48 | NA |
| YDDPEVQK(ac)DIK (SEQ ID NO: 578) | HSPA9 | NONE | NONE | NONE | NONE | NONE | NONE |
| DDNGK(ac)PYVLPSVR (SEQ ID NO: 579) | GOT2 | NONE | NONE | NONE | NONE | NONE | NONE |
| AGEATVK(ac)FLK (SEQ ID NO: 580) | MRPL9 | NONE | NONE | NONE | NONE | NONE | NONE |
| RNPDTQWITK(ac)PVHK (SEQ ID NO: 581) | RPL15 | NONE | NONE | NONE | NONE | NONE | NONE |
| LQDFK(ac)SFLLK (SEQ ID NO: 582) | SHMT2 | NONE | NONE | NONE | NONE | NONE | NONE |
| EFALK(ac)HLPNDPMFK (SEQ ID NO: 583) | CS | NONE | NONE | NONE | NONE | NONE | NONE |
| TACAINK(ac)VLMGR (SEQ ID NO: 584) | FAM98A | NONE | NONE | NONE | NONE | NONE | NONE |
| LMK(ac)YLLEQLK (SEQ ID NO: 585) | MB21D1 | NONE | NONE | NONE | NONE | NONE | NONE |
| ASEIGEK(ac)YR (SEQ ID NO: 586) | KIAA0947 | NONE | NONE | NONE | NONE | NONE | NONE |
| ITK(ac)SDGIR (SEQ ID NO: 587) | SLC25A6 | NONE | NONE | NONE | NONE | NONE | NONE |
| ELEAENYHDIK(ac)R (SEQ ID NO: 588) | SPTBN1 | NONE | NONE | NONE | NONE | NONE | NONE |
| NLHSK(ac)WLK (SEQ ID NO: 589) | SPTBN1 | NONE | NONE | NONE | NONE | NONE | NONE |
| LTSLNVK(ac)YNNDK (SEQ ID NO: 590) | ROD1 | NONE | NONE | NONE | NONE | NONE | NONE |
| VASK(ac)QLEEEDGSR (SEQ ID NO: 591) | COPG | NONE | NONE | NONE | NONE | NONE | NONE |
| TAWLDGK(ac)HVVFGK (SEQ ID NO: 592) | PPIB | NONE | NONE | NONE | NONE | NONE | NONE |
| VWNYK(ac)LR (SEQ ID NO: 593) | COPA | NONE | NONE | NONE | NONE | NONE | NONE |
| K(ac)NINCSIEESFQR (SEQ ID NO: 594) | HMGCL | NONE | NONE | NONE | NONE | NONE | NONE |
| YEK(ac)DIAAYR (SEQ ID NO: 595) | HMGB2 | NONE | NONE | NONE | NONE | NONE | NONE |
| FYK(ac)DVLEVGELAK (SEQ ID NO: 596) | NAA50 | − | − | + | 0.12 | −0.02 | 0.52 |
| VVK(ac)QASEGPLK (SEQ ID NO: 597) | GAPDH | NONE | NONE | NONE | NONE | NONE | NONE |
| TSSAFVGK(ac)TPEASPEPK | PSMD1 | NONE | NONE | NONE | NONE | NONE | NONE |
| (SEQ ID NO: 598) | | | | | | | |
| ADGIVSK(ac)NF (SEQ ID NO: 599) | RPS21 | NONE | NONE | NONE | NONE | NONE | NONE |
| ISEQSDAK(ac)LK (SEQ ID NO: 600) | ATP5A1 | NONE | NONE | NONE | NONE | NONE | NONE |
| K(ac)SSETLR (SEQ ID NO: 601) | CNP | NONE | NONE | NONE | NONE | NONE | NONE |
| SGLLDK(ac)WK (SEQ ID NO: 602) | LOC653566, | NONE | NONE | NONE | NONE | NONE | NONE |
| | SPCS2 | | | | | | |
| LNLDK(ac)MMEQK (SEQ ID NO: 603) | DLD | NONE | NONE | NONE | NONE | NONE | NONE |
| LAK(ac)YFFNK (SEQ ID NO: 604) | PWP2 | NONE | NONE | NONE | NONE | NONE | NONE |
| NSFDCFK(ac)K (SEQ ID NO: 605) | SLC25A13 | NONE | NONE | NONE | NONE | NONE | NONE |
| AGTFK(ac)MVFTPK (SEQ ID NO: 606) | IDH2 | NONE | NONE | NONE | NONE | NONE | NONE |
| SLNLK(ac)HIK (SEQ ID NO: 607) | SNORD84, | NONE | NONE | NONE | NONE | NONE | NONE |
| | DDX39B | | | | | | |
| DHCVAHK(ac)LFNNLK (SEQ ID NO: 608) | UQCRH | NONE | NONE | NONE | NONE | NONE | NONE |
| NVK(ac)FVLK (SEQ ID NO: 609) | SSR3 | NONE | NONE | NONE | NONE | NONE | NONE |
| VLTVINQTQK(ac)ENLR (SEQ ID NO: 610) | RPL35 | NONE | NONE | NONE | NONE | NONE | NONE |
| GAK(ac)PVVVLQK (SEQ ID NO: 611) | NIPBL | NONE | NONE | NONE | NONE | NONE | NONE |
| ELEK(ac)VCNPIITK (SEQ ID NO: 612) | HSPA8 | NONE | NONE | NONE | NONE | NONE | NONE |
| IHDVLCK(ac)LVEK (SEQ ID NO: 613) | LRPPRC | NONE | NONE | NONE | NONE | NONE | NONE |
| K(ac)FAYLGR (SEQ ID NO: 614) | SNORD32A, | NONE | NONE | NONE | NONE | NONE | NONE |
| | RPL13A, | | | | | | |
| | SNORD33, | | | | | | |
| | SNORD34 | | | | | | |
| SK(ac)GK(ac)NSDEEAPK | ING4 | NONE | NONE | NONE | NONE | NONE | NONE |
| (SEQ ID NO: 615) | | | | | | | |
| K(ac)EYVFADSK (SEQ ID NO: 616) | INPP5D, | NONE | NONE | NONE | NONE | NONE | NONE |
| | LOC646743 | | | | | | |
| EIAENALGK(ac)HK (SEQ ID NO: 617) | HNRNPH3 | NONE | NONE | NONE | NONE | NONE | NONE |
| FQK(ac)ELER (SEQ ID NO: 618) | MRPL44 | NONE | NONE | NONE | NONE | NONE | NONE |
| GFSLLATEDK(ac)EALKK (SEQ ID NO: 619) | PARP1 | NONE | NONE | NONE | NONE | NONE | NONE |
| VIISAPSADAPMFVMGVNHEK(ac)YDNSLK | GAPDH | NONE | NONE | NONE | NONE | NONE | NONE |
| (SEQ ID NO: 620) | | | | | | | |
| GAGTDEK(ac)TLTR (SEQ ID NO: 621) | ANXA6 | NONE | NONE | NONE | NONE | NONE | NONE |
| GPLDK(ac)WR (SEQ ID NO: 622) | RPL10L | NONE | NONE | NONE | NONE | NONE | NONE |
| EFHQAGK(ac)PIGLCCIAPVLAAK | C21orf33 | NONE | NONE | NONE | NONE | NONE | NONE |
| (SEQ ID NO: 623) | | | | | | | |
| TTLLYK(ac)LK (SEQ ID NO: 624) | ARL11 | NONE | NONE | NONE | NONE | NONE | NONE |
| VPNDK(ac)YYGAQTVR (SEQ ID NO: 625) | FH | NONE | NONE | NONE | NONE | NONE | NONE |
| ADTLTPEECQQFK(ac)K (SEQ ID NO: 626) | 7-Sep | NONE | NONE | NONE | NONE | NONE | NONE |
| AFAK(ac)ELFLGK (SEQ ID NO: 627) | ACAD9 | NONE | NONE | NONE | NONE | NONE | NONE |
| SYVSEK(ac)DVTSAK (SEQ ID NO: 628) | LRPPRC | NONE | NONE | NONE | NONE | NONE | NONE |
| LIGK(ac)QGR (SEQ ID NO: 629) | AKAP1 | NONE | NONE | NONE | NONE | NONE | NONE |
| EHTALLK(ac)IEGVYAR (SEQ ID NO: 630) | RPL35A | NONE | NONE | NONE | NONE | NONE | NONE |
| FNDGSDEK(ac)KK (SEQ ID NO: 631) | ATP5A1 | NONE | NONE | NONE | NONE | NONE | NONE |
| FSK(ac)SQLDIIIHSLK (SEQ ID NO: 632) | XRCC5 | NONE | NONE | NONE | NONE | NONE | NONE |
| LTCEEEEEK(ac)IFGR (SEQ ID NO: 633) | SMARCA2 | NONE | NONE | NONE | NONE | NONE | NONE |
| NDEELNK(ac)LLGR (SEQ ID NO: 634) | HIST1H2AE, | NONE | NONE | NONE | NONE | NONE | NONE |
| | HIST1H2AB, | | | | | | |
| | HIST1H2AD, | | | | | | |
| | HIST1H2AG, | | | | | | |
| | HIST1H2AK, | | | | | | |
| | HIST1H2AL, | | | | | | |
| | HIST1H2AM, | | | | | | |
| | HIST1H2AJ, | | | | | | |
| | HIST1H2AI | | | | | | |
| ALEAVFGK(ac)YGR (SEQ ID NO: 635) | RBMX | NONE | NONE | NONE | NONE | NONE | NONE |
| TFK(ac)TVFAEHISDECK (SEQ ID NO: 636) | SNORD43, | NONE | NONE | NONE | NONE | NONE | NONE |
| | RPL3 | | | | | | |
| YK(ac)PESEELTAER (SEQ ID NO: 637) | P4HB | NONE | NONE | NONE | NONE | NONE | NONE |
| NAEK(ac)YAEEDRR (SEQ ID NO: 638) | HSPA9 | NONE | NONE | NONE | NONE | NONE | NONE |
| SKSDPIMLLK(ac)DR (SEQ ID NO: 639) | PDHA1 | NONE | NONE | NONE | NONE | NONE | NONE |
| GVDPK(ac)FLR (SEQ ID NO: 640) | RPL29, | NONE | NONE | NONE | NONE | NONE | NONE |
| | RPL29P4 | | | | | | |
| LIK(ac)DGLIIR (SEQ ID NO: 641) | RPL19 | NONE | NONE | NONE | NONE | NONE | NONE |
| HLLPK(ac)IR (SEQ ID NO: 642) | PGD | NONE | NONE | NONE | NONE | NONE | NONE |
| LGK(ac)PSLVR (SEQ ID NO: 643) | ATAD3B | NONE | NONE | NONE | NONE | NONE | NONE |
| EQIYK(ac)LAK (SEQ ID NO: 644) | RPS13 | NONE | NONE | NONE | NONE | NONE | NONE |
| VEYFLK(ac)WK (SEQ ID NO: 645) | CBX3 | NONE | NONE | NONE | NONE | NONE | NONE |
| HYK(ac)TDFDK (SEQ ID NO: 646) | IDH2 | NONE | NONE | NONE | NONE | NONE | NONE |
| EGYAWAEDK(ac)EHCEEYGR | C22orf28 | NONE | NONE | NONE | NONE | NONE | NONE |
| (SEQ ID NO: 647) | | | | | | | |
| LAYIAHPK(ac)LGK (SEQ ID NO: 648) | RPL29, | NONE | NONE | NONE | NONE | NONE | NONE |
| | RPL29P4 | | | | | | |
| IFSQETLTK(ac)AQILK (SEQ ID NO: 649) | PCYOX1 | NONE | NONE | NONE | NONE | NONE | NONE |
| ALTGGIAHLFK(ac)QNK (SEQ ID NO: 650) | DLD | − | − | + | 0.18 | 0.07 | 0.42 |
| FYVK(ac)DHR (SEQ ID NO: 651) | CISD1 | NONE | NONE | NONE | NONE | NONE | NONE |
| SPVNTK(ac)SLFK (SEQ ID NO: 652) | PRKDC | NONE | NONE | NONE | NONE | NONE | NONE |
| HIVK(ac)EFK (SEQ ID NO: 653) | HSPA9 | NONE | NONE | NONE | NONE | NONE | NONE |
| MYKEEGLK(ac)AFYK (SEQ ID NO: 654) | SLC25A3 | NONE | NONE | NONE | NONE | NONE | NONE |
| LDAQVK(ac)ELVLK (SEQ ID NO: 655) | RPN1 | NONE | NONE | NONE | NONE | NONE | NONE |
| GYPTLK(ac)LFKPGQEAVK | TXNDC5, | NONE | NONE | NONE | NONE | NONE | NONE |
| (SEQ ID NO: 656) | MUTED, | | | | | | |
| | MUTED- | | | | | | |
| | TXNDC5 | | | | | | |
| EVCFACVDGK(ac)EFR (SEQ ID NO: 657) | CLTC | NONE | NONE | NONE | NONE | NONE | NONE |
| YK(ac)EALEQCR (SEQ ID NO: 658) | TAP2 | NONE | NONE | NONE | NONE | NONE | NONE |
| YELTGK(ac)FER (SEQ ID NO: 659) | ANXA6 | NONE | NONE | NONE | NONE | NONE | NONE |
| FIK(ac)IDGK (SEQ ID NO: 660) | RPS4X | NONE | NONE | NONE | NONE | NONE | NONE |
| LAMVK(ac)AEPDVK (SEQ ID NO: 661) | NDUFA5 | NONE | NONE | NONE | NONE | NONE | NONE |
| NEK(ac)LFYR (SEQ ID NO: 662) | ME3 | NONE | NONE | NONE | NONE | NONE | NONE |
| K(ac)FVCPECSK (SEQ ID NO: 663) | SP3 | NONE | NONE | NONE | NONE | NONE | NONE |
| DLSLDDFK(ac)GK (SEQ ID NO: 664) | PRDX3 | NONE | NONE | NONE | NONE | NONE | NONE |
| DLTDYLMK(ac)ILTER (SEQ ID NO: 665) | ACTBL2 | NONE | NONE | NONE | NONE | NONE | NONE |
| HGASPELQK(ac)QIR (SEQ ID NO: 666) | PEX11B | NONE | NONE | NONE | NONE | NONE | NONE |
| LNQLK(ac)PGLQYK (SEQ ID NO: 667) | ILF3 | NONE | NONE | NONE | NONE | NONE | NONE |
| ISK(ac)LYGDLK (SEQ ID NO: 668) | SDHA | NONE | NONE | NONE | NONE | NONE | NONE |
| LTSDDVK(ac)EQIYK (SEQ ID NO: 669) | RPS13 | NONE | NONE | NONE | NONE | NONE | NONE |
| SCNCLLLK(ac)VNQIGSVIESLQACK | ENO1 | NONE | NONE | NONE | NONE | NONE | NONE |
| (SEQ ID NO: 670) | | | | | | | |
| VTCIDPNPNFEK(ac)FLIK | METTL7A | + | − | − | 0.44 | 0.27 | 0.18 |
| (SEQ ID NO: 671) | | | | | | | |
| LILGLMMPPAHYDAK(ac)QLK | ANXA6 | NONE | NONE | NONE | NONE | NONE | NONE |
| (SEQ ID NO: 672) | | | | | | | |
| EVK(ac)LLLLGAGESGK (SEQ ID NO: 673) | GNAI3 | NONE | NONE | NONE | NONE | NONE | NONE |
| EAKPDELMDSK(ac)LR (SEQ ID NO: 674) | VAPA | NONE | NONE | NONE | NONE | NONE | NONE |
| YNWSAK(ac)AK (SEQ ID NO: 675) | RPL37 | NONE | NONE | NONE | NONE | NONE | NONE |
| GNK(ac)PWISLPR (SEQ ID NO: 676) | RPS4X | NONE | NONE | NONE | NONE | NONE | NONE |
| EVVEAHVDQK(ac)NK (SEQ ID NO: 677) | C21orf33 | NONE | NONE | NONE | NONE | NONE | NONE |
| LENDK(ac)SFR (SEQ ID NO: 678) | ECI1 | − | + | − | 0.12 | −0.38 | NA |
| LVAMK(ac)FLR (SEQ ID NO: 679) | NME1, | NONE | NONE | NONE | NONE | NONE | NONE |
| | NME2, | | | | | | |
| | NME1-NME2 | | | | | | |
| KVPFGK(ac)TYCCDLK (SEQ ID NO: 680) | MTCH2 | NONE | NONE | NONE | NONE | NONE | NONE |
| TK(ac)FVDGVSTVAR (SEQ ID NO: 681) | SMC2 | NONE | NONE | NONE | NONE | NONE | NONE |
| LAQLEEAK(ac)QASIQHIQNAIDTEK | ATP5F1 | NONE | NONE | NONE | NONE | NONE | NONE |
| (SEQ ID NO: 682) | | | | | | | |
| IPVPVQK(ac)NIDQQIK (SEQ ID NO: 683) | PRPF38B | NONE | NONE | NONE | NONE | NONE | NONE |
| YQLDKDGVVLFK(ac)K (SEQ ID NO: 684) | P4HB | NONE | NONE | NONE | NONE | NONE | NONE |
| VDIRPQLLK(ac)NALQR (SEQ ID NO: 685) | HP1BP3 | NONE | NONE | NONE | NONE | NONE | NONE |
| NQK(ac)LTATTQK (SEQ ID NO: 686) | TPR | NONE | NONE | NONE | NONE | NONE | NONE |
| YYK(ac)NIGLGFK (SEQ ID NO: 687) | RPS11 | NONE | NONE | NONE | NONE | NONE | NONE |
| SGK(ac)YDLDFK (SEQ ID NO: 688) | ENO1 | NONE | NONE | NONE | NONE | NONE | NONE |
| LYGDLK(ac)HLK (SEQ ID NO: 689) | SDHA | NONE | NONE | NONE | NONE | NONE | NONE |
| VENEAEK(ac)DLQCHAPVR | BRD7 | NONE | NONE | NONE | NONE | NONE | NONE |
| (SEQ ID NO: 690) | | | | | | | |
| DSLYK(ac)DAMQYASESK (SEQ ID NO: 691) | CLTC | NONE | NONE | NONE | NONE | NONE | NONE |
| TISAK(ac)YAEER (SEQ ID NO: 692) | MYH9 | NONE | NONE | NONE | NONE | NONE | NONE |
| (ac)ASNK(ac)TTLQK (SEQ ID NO: 693) | CBX3 | NONE | NONE | NONE | NONE | NONE | NONE |
| GYLADPAK(ac)FPEAR (SEQ ID NO: 694) | MRPL15 | NONE | NONE | NONE | NONE | NONE | NONE |
| SCEIK(ac)FLTFK (SEQ ID NO: 695) | MRPL39 | NONE | NONE | NONE | NONE | NONE | NONE |
| SELK(ac)HAK (SEQ ID NO: 696) | ALDH18A1 | NONE | NONE | NONE | NONE | NONE | NONE |
| SCGLNPK(ac)LEK (SEQ ID NO: 697) | ATRX | NONE | NONE | NONE | NONE | NONE | NONE |
| EVK(ac)VLLDQLR (SEQ ID NO: 698) | C20orf3 | NONE | NONE | NONE | NONE | NONE | NONE |
| KAVTK(ac)VQK (SEQ ID NO: 699) | HIST2H2BF | NONE | NONE | NONE | NONE | NONE | NONE |
| ILQK(ac)QGFK (SEQ ID NO: 700) | DLD | NONE | NONE | NONE | NONE | NONE | NONE |
| NK(ac)FGAPQK (SEQ ID NO: 701) | HADHA | NONE | NONE | NONE | NONE | NONE | NONE |
| VTAVHK(ac)ANIMK (SEQ ID NO: 702) | IDH3G | NONE | NONE | NONE | NONE | NONE | NONE |
| ACPEK(ac)HFAFK (SEQ ID NO: 703) | FAM3C | NONE | NONE | NONE | NONE | NONE | NONE |
| KPTLDK(ac)PSPETFVK (SEQ ID NO: 704) | HSD17B12 | NONE | NONE | NONE | NONE | NONE | NONE |
| QLAQK(ac)YNCDK (SEQ ID NO: 705) | UBA52 | NONE | NONE | NONE | NONE | NONE | NONE |
| YTAQVDAEEK(ac)EDVK (SEQ ID NO: 706) | ATP5H | NONE | NONE | NONE | NONE | NONE | NONE |
| PLISPQTSHK(ac)TLSK (SEQ ID NO: 707) | PDZD2 | NONE | NONE | NONE | NONE | NONE | NONE |
| ADK(ac)LAEEHSS (SEQ ID NO: 708) | ATP5B | NONE | NONE | NONE | NONE | NONE | NONE |
| DFGSFDK(ac)FK (SEQ ID NO: 709) | SOD2 | − | + | − | −0.28 | −0.48 | NA |
| KIDK(ac)YTEVLK (SEQ ID NO: 710) | SNORD32A, | NONE | NONE | NONE | NONE | NONE | NONE |
| | RPL13A, | | | | | | |
| | SNORD33, | | | | | | |
| | SNORD34 | | | | | | |
| CSK(ac)LPSSTK(ac)SGWPR | PHF15 | − | − | + | 0.11 | 0.04 | 0.43 |
| (SEQ ID NO: 711) | | | | | | | |
| KAINK(ac)AQK (SEQ ID NO: 712) | HIST1H2BM | NONE | NONE | NONE | NONE | NONE | NONE |
| EFPEK(ac)QELHR (SEQ ID NO: 713) | ZER1 | NONE | NONE | NONE | NONE | NONE | NONE |
| TDLEK(ac)DIISDTSGDFR | ANXA2P2 | NONE | NONE | NONE | NONE | NONE | NONE |
| (SEQ ID NO: 714) | | | | | | | |
| TPAQYDASELK(ac)ASMK (SEQ ID NO: 715) | ANXA2P2 | NONE | NONE | NONE | NONE | NONE | NONE |
| LTCYLCK(ac)QK (SEQ ID NO: 716) | BRD1 | NONE | NONE | NONE | NONE | NONE | NONE |
| AINK(ac)AQK(ac)K (SEQ ID NO: 717) | HIST1H2BM | NONE | NONE | NONE | NONE | NONE | NONE |
| KGIEK(ac)NLGIGK (SEQ ID NO: 718) | MDH2 | NONE | NONE | NONE | NONE | NONE | NONE |
| DNGK(ac)HALIIYDDLSK (SEQ ID NO: 719) | ATP5A1 | NONE | NONE | NONE | NONE | NONE | NONE |
| QLYNIYAK(ac)HTK (SEQ ID NO: 720) | CLPP | NONE | NONE | NONE | NONE | NONE | NONE |
| GKGDK(ac)AQIEK (SEQ ID NO: 721) | HSPD1 | NONE | NONE | NONE | NONE | NONE | NONE |
| K(ac)FLVHNVK (SEQ ID NO: 722) | RPL32 | NONE | NONE | NONE | NONE | NONE | NONE |
| GLIK(ac)LVSK (SEQ ID NO: 723) | RPS25 | NONE | NONE | NONE | NONE | NONE | NONE |
| IGFAEK(ac)VAAK (SEQ ID NO: 724) | FH | NONE | NONE | NONE | NONE | NONE | NONE |
| (ac)AEK(ac)FDCHYCR (SEQ ID NO: 725) | FHL1 | NONE | NONE | NONE | NONE | NONE | NONE |
| GALPLDTVTFYK(ac)VIPK | ERP29 | NONE | NONE | NONE | NONE | NONE | NONE |
| (SEQ ID NO: 726) | | | | | | | |
| HWGGNVLGPK(ac)SVAR (SEQ ID NO: 727) | RPL7A, | NONE | NONE | NONE | NONE | NONE | NONE |
| | SNORD24 | | | | | | |
| TITMK(ac)QLLR (SEQ ID NO: 728) | BCAT2 | NONE | NONE | NONE | NONE | NONE | NONE |
| SK(ac)FVLVK (SEQ ID NO: 729) | ERP29 | NONE | NONE | NONE | NONE | NONE | NONE |
| LVEDTK(ac)HRPK (SEQ ID NO: 730) | MRPL9 | NONE | NONE | NONE | NONE | NONE | NONE |
| TVGQLYK(ac)EQLAK (SEQ ID NO: 731) | MYH9 | NONE | NONE | NONE | NONE | NONE | NONE |
| AAETCQEPK(ac)EFR (SEQ ID NO: 732) | NDUFAF4 | NONE | NONE | NONE | NONE | NONE | NONE |
| VVLIGDSGVGK(ac)SNLLSR | RAB11B | NONE | NONE | NONE | NONE | NONE | NONE |
| (SEQ ID NO: 733) | | | | | | | |
| SGK(ac)YVLGYK (SEQ ID NO: 734) | RPL30 | NONE | NONE | NONE | NONE | NONE | NONE |
| ILMEHIHK(ac)LK (SEQ ID NO: 735) | RPL19 | NONE | NONE | NONE | NONE | NONE | NONE |
| EK(ac)VFYSLMR (SEQ ID NO: 736) | EPHX1 | NONE | NONE | NONE | NONE | NONE | NONE |
| K(ac)FDQLLAEEK (SEQ ID NO: 737) | MYH9 | NONE | NONE | NONE | NONE | NONE | NONE |
| FVEFDMK(ac)PVCK (SEQ ID NO: 738) | LIMS2 | NONE | NONE | NONE | NONE | NONE | NONE |
| K(ac)GFITVDGR (SEQ ID NO: 739) | LAMA1 | NONE | NONE | NONE | NONE | NONE | NONE |
| KAEAQIAAK(ac)NLDK (SEQ ID NO: 740) | GOT2 | NONE | NONE | NONE | NONE | NONE | NONE |
| DK(ac)QFVAK (SEQ ID NO: 741) | SND1 | NONE | NONE | NONE | NONE | NONE | NONE |
| AGACNTVIK(ac)QLMR (SEQ ID NO: 742) | ATP2A3 | NONE | NONE | NONE | NONE | NONE | NONE |
| KQQAK(ac)FDECVLDK (SEQ ID NO: 743) | NDUFA8 | NONE | NONE | NONE | NONE | NONE | NONE |
| ALCTGEK(ac)GFGYK (SEQ ID NO: 744) | PPIF | NONE | NONE | NONE | NONE | NONE | NONE |
| IYK(ac)SDGIK (SEQ ID NO: 745) | SLC25A5 | NONE | NONE | NONE | NONE | NONE | NONE |
| DEGGK(ac)AFFK (SEQ ID NO: 746) | SLC25A5 | NONE | NONE | NONE | NONE | NONE | NONE |
| SYLK(ac)EFR (SEQ ID NO: 747) | TECR | NONE | NONE | NONE | NONE | NONE | NONE |
| VPK(ac)TAENFR (SEQ ID NO: 748) | PPID | NONE | NONE | NONE | NONE | NONE | NONE |
| VNCLDK(ac)FWHK (SEQ ID NO: 749) | LASP1 | NONE | NONE | NONE | NONE | NONE | NONE |
| KGEDFVK(ac)TLK (SEQ ID NO: 750) | MDH2 | − | + | − | 0.15 | 0.49 | NA |
| EAEFTK(ac)SIAK (SEQ ID NO: 751) | LOC653566, | NONE | NONE | NONE | NONE | NONE | NONE |
| | SPCS2 | | | | | | |
| SGYYK(ac)VLGK (SEQ ID NO: 752) | RPL27A | NONE | NONE | NONE | NONE | NONE | NONE |
| YVLGYK(ac)QTLK (SEQ ID NO: 753) | RPL30 | NONE | NONE | NONE | NONE | NONE | NONE |
| FEDK(ac)TVAYTEQK (SEQ ID NO: 754) | PDIA3 | NONE | NONE | NONE | NONE | NONE | NONE |
| IVNALK(ac)SQVDK (SEQ ID NO: 755) | OAT | NONE | NONE | NONE | NONE | NONE | NONE |
| ILEFFGLK(ac)K (SEQ ID NO: 756) | P4HB | NONE | NONE | NONE | NONE | NONE | NONE |
| LCGQDLNK(ac)TSR (SEQ ID NO: 757) | DPAGT1 | NONE | NONE | NONE | NONE | NONE | NONE |
| VYFK(ac)DTHPK (SEQ ID NO: 758) | CYB5R3 | NONE | NONE | NONE | NONE | NONE | NONE |
| ILSK(ac)LSEETK (SEQ ID NO: 759) | UFL1 | NONE | NONE | NONE | NONE | NONE | NONE |
| ATWK(ac)SNYFLK (SEQ ID NO: 760) | RPLP0 | NONE | NONE | NONE | NONE | NONE | NONE |
| AVDSQILPK(ac)IK (SEQ ID NO: 761) | RPL6 | NONE | NONE | NONE | NONE | NONE | NONE |
| EK(ac)LQEEMLQR (SEQ ID NO: 762) | VIM | NONE | NONE | NONE | NONE | NONE | NONE |
| MIYASSK(ac)DAIKK (SEQ ID NO: 763) | CFL1 | NONE | NONE | NONE | NONE | NONE | NONE |
| YNDPIYVK(ac)LEK (SEQ ID NO: 764) | AP1B1 | NONE | NONE | NONE | NONE | NONE | NONE |
| VTDDLVCLVYK(ac)TDQAQDVK | SRP9 | NONE | NONE | NONE | NONE | NONE | NONE |
| (SEQ ID NO: 765) | | | | | | | |
| TVVNK(ac)DVFR (SEQ ID NO: 766) | RPL27 | NONE | NONE | NONE | NONE | NONE | NONE |
| NLQEAEEWYK(ac)SK (SEQ ID NO: 767) | PRPH | NONE | NONE | NONE | NONE | NONE | NONE |
| Mac)FGGPGAR (SEQ ID NO: 768) | RPS16 | NONE | NONE | NONE | NONE | NONE | NONE |
| LLLLGAGESGK(ac)STIVK | GNAL | NONE | NONE | NONE | NONE | NONE | NONE |
| (SEQ ID NO: 769) | | | | | | | |
| SFVK(ac)VYNYNHLMPTR (SEQ ID NO: 770) | RPL27 | NONE | NONE | NONE | NONE | NONE | NONE |
| YVK(ac)ALFR (SEQ ID NO: 771) | TOMM70A | NONE | NONE | NONE | NONE | NONE | NONE |
| LEPLK(ac)PLK (SEQ ID NO: 772) | PRR12 | NONE | NONE | NONE | NONE | NONE | NONE |
| VGDK(ac)VLLPEYGGTK (SEQ ID NO: 773) | HSPE1 | NONE | NONE | NONE | NONE | NONE | NONE |
| GGVVGIK(ac)VDK (SEQ ID NO: 774) | ALDOA | NONE | NONE | NONE | NONE | NONE | NONE |
| VAFK(ac)YCQK (SEQ ID NO: 775) | ALG5 | NONE | NONE | NONE | NONE | NONE | NONE |
| SVPHLQK(ac)VFDR (SEQ ID NO: 776) | ANXA2P2 | NONE | NONE | NONE | NONE | NONE | NONE |
| VYK(ac)EMYK (SEQ ID NO: 777) | ANXA2P2 | NONE | NONE | NONE | NONE | NONE | NONE |
| SELTGK(ac)FEK (SEQ ID NO: 778) | ANXA5 | − | + | − | 0.31 | 0.40 | NA |
| EFIEK(ac)YDK (SEQ ID NO: 779) | ANXA6 | NONE | NONE | NONE | NONE | NONE | NONE |
| ESCSK(ac)HASGTSFHLPQPSFR | BRPF3 | NONE | NONE | NONE | NONE | NONE | NONE |
| (SEQ ID NO: 780) | | | | | | | |
| KAEAAVVAVAEK(ac)R (SEQ ID NO: 781) | C11orf31 | NONE | NONE | NONE | NONE | NONE | NONE |
| VNK(ac)EVER (SEQ ID NO: 782) | C21orf33 | NONE | NONE | NONE | NONE | NONE | NONE |
| GFSIPECQK(ac)LLPK (SEQ ID NO: 783) | CS | NONE | NONE | NONE | NONE | NONE | NONE |
| CINIILK(ac)NDK (SEQ ID NO: 784) | CXorf59 | NONE | NONE | NONE | NONE | NONE | NONE |
| NEEATK(ac)HLECTK (SEQ ID NO: 785) | FXR1 | NONE | NONE | NONE | NONE | NONE | NONE |
| AGK(ac)DSGMac)AK (SEQ ID NO: 786) | H2AFV | NONE | NONE | NONE | NONE | NONE | NONE |
| ESK(ac)ALMGLYHGQVLCK | HADHA | NONE | NONE | NONE | NONE | NONE | NONE |
| (SEQ ID NO: 787) | | | | | | | |
| FGAPQK(ac)DVK (SEQ ID NO: 788) | HADHA | NONE | NONE | NONE | NONE | NONE | NONE |
| ILQEGVDPK(ac)K (SEQ ID NO: 789) | HADHA | NONE | NONE | NONE | NONE | NONE | NONE |
| GSK(ac)K(ac)AITK (SEQ ID NO: 790) | HIST1H2BB | NONE | NONE | NONE | NONE | NONE | NONE |
| PEPTK(ac)SAPAPK(ac)K | HIST1H2BD | NONE | NONE | NONE | NONE | NONE | NONE |
| (SEQ ID NO: 791) | | | | | | | |
| GSK(ac)K(ac)AINK (SEQ ID NO: 792) | HIST1H2BM | NONE | NONE | NONE | NONE | NONE | NONE |
| K(ac)AINK(ac)AQK (SEQ ID NO: 793) | HIST1H2BM | − | + | − | NA | −0.97 | 0.27 |
| K(ac)AVTK(ac)AQK (SEQ ID NO: 794) | HIST2H2BE | NONE | NONE | NONE | NONE | NONE | NONE |
| PEPAK(ac)SAPAPKK (SEQ ID NO: 795) | HIST2H2BE | NONE | NONE | NONE | NONE | NONE | NONE |
| K(ac)QLATK(ac)AAR (SEQ ID NO: 796) | HIST2H3D, | NONE | NONE | NONE | NONE | NONE | NONE |
| | HIST2H3A, | | | | | | |
| | HIST2H3C | | | | | | |
| TEMDWVLK(ac)HSGPNSADSANDGFVR | HNRNPF | NONE | NONE | NONE | NONE | NONE | NONE |
| (SEQ ID NO: 797) | | | | | | | |
| ADLIK(ac)QYGR (SEQ ID NO: 798) | HSDL1 | NONE | NONE | NONE | NONE | NONE | NONE |
| FYEQFSK(ac)NIK (SEQ ID NO: 799) | HSP90AA1 | NONE | NONE | NONE | NONE | NONE | NONE |
| LMK(ac)ICMNEDPAK (SEQ ID NO: 800) | ILK | NONE | NONE | NONE | NONE | NONE | NONE |
| (ac)ASPSLERPEK(ac)GAGK | KRR1 | NONE | NONE | NONE | NONE | NONE | NONE |
| (SEQ ID NO: 801) | | | | | | | |
| INKALDK(ac)TK (SEQ ID NO: 802) | MYH9 | − | + | − | NA | 4.75 | NA |
| IANILK(ac)DKDPPIPVAK | PDIA4 | NONE | NONE | NONE | NONE | NONE | NONE |
| (SEQ ID NO: 803) | | | | | | | |
| NTEDLQCYVK(ac)PTK (SEQ ID NO: 804) | PHF16 | NONE | NONE | NONE | NONE | NONE | NONE |
| VDEVK(ac)STIK (SEQ ID NO: 805) | RPL10A | NONE | NONE | NONE | NONE | NONE | NONE |
| KLQK(ac)AALLK (SEQ ID NO: 806) | RPL14 | NONE | NONE | NONE | NONE | NONE | NONE |
| YSVDIPLDK(ac)TVVNK (SEQ ID NO: 807) | RPL27 | NONE | NONE | NONE | NONE | NONE | NONE |
| LNK(ac)AVWAK (SEQ ID NO: 808) | RPL31 | NONE | NONE | NONE | NONE | NONE | NONE |
| K(ac)FGQGSR (SEQ ID NO: 809) | RPS29 | NONE | NONE | NONE | NONE | NONE | NONE |
| DQTK(ac)SIVEK (SEQ ID NO: 810) | RTN3 | NONE | NONE | NONE | NONE | NONE | NONE |
| YMVK(ac)SCGK (SEQ ID NO: 811) | SNORA70, | NONE | NONE | NONE | NONE | NONE | NONE |
| | RPL10 | | | | | | |
| LAHEVGWK(ac)YQAVTATLEEK | SNORD32A, | NONE | NONE | NONE | NONE | NONE | NONE |
| (SEQ ID NO: 812) | RPL13A, | | | | | | |
| | SNORD33, | | | | | | |
| | SNORD34 | | | | | | |
| ALEK(ac)IDLK (SEQ ID NO: 813) | SNORD43, | NONE | NONE | NONE | NONE | NONE | NONE |
| | RPL3 | | | | | | |
| AMMEK(ac)LVK(ac)SISQLK | STAT2 | NONE | NONE | NONE | NONE | NONE | NONE |
| (SEQ ID NO: 814) | | | | | | | |
| LLGITK(ac)ECVMR (SEQ ID NO: 815) | TLN1 | NONE | NONE | NONE | NONE | NONE | NONE |
| GK(ac)GK(ac)AGR (SEQ ID NO: 816) | TPPP | NONE | NONE | NONE | NONE | NONE | NONE |
| LGDVISIQPCPDVK(ac)YGK | VCP | NONE | NONE | NONE | NONE | NONE | NONE |
| (SEQ ID NO: 817) | | | | | | | |

### Example 3

### Biological Pathway Analysis

This example describes the biological pathway analysis that was performed on each of Compound A, Compound B, and Tubastatin A.

Once biomarker peptides were identified for a specific drug (either Compound A, Compound B, or Tubastatin A) by the SILAC method in Example 1, the biomarker peptides were compared to peptides/proteins in the biological pathway database at www dot broadinstitute dot org/gsea/index dot jsp in order to identify major biological pathways or functions implicated by the drug's action.

The results of the biological pathway analysis for each of Compound B, Tubastatin A, and Compound A, are in Tables 3, 5, and 7, respectively. Only the top 50 pathways identified for each drug are listed in each of Tables 3, 5, and 7.

The first column of Tables 3, 5, and 7 shows the name of the pathway implicated.

The second column of Tables 3, 5, and 7 shows the number of genes in the pathway observed for each drug treated group of cells.

The third column of Tables 3, 5, and 7 shows the total number of genes in the pathway stated in the first column.

The fourth column of Tables 3, 5, and 7 shows the ratio of the number in the second column to the number in the third column.

The fifth column of Tables 3, 5, and 7 shows the p-value for the numbers in the second and third columns.

The sixth column of Tables 3, 5, and 7 gives a description of the pathway identified in the first column.

TABLE 3
(Compound B (+))

| Pathway | Observed | Pathway_size | Ratio | P-value | Description | Code |
|---|---|---|---|---|---|---|
| PUJANA_BRCA1_ PCC _ | 68 | 1651 | 0.041187 | 1.19E−52 | Genes constituting the BRCA1-PCC | A |
| NETWORK | | | | | network of transcripts whose ex- | |
| | | | | | pression positively correlated | |
| | | | | | (Pearson correlation coefficient, | |
| | | | | | PCC ≧ 0.4) with that of BRCA1 | |
| | | | | | <ahref=‘http://www.ncbi.nlm.nih. | |
| | | | | | gov/gene/672’>[GeneID=672]</a> | |
| | | | | | across a compendium of normal | |
| | | | | | tissues. | |
| DIAZ_CHRONIC_ | 58 | 1382 | 0.041968 | 6.25E−45 | Genes up-regulated in CD34+ | D |
| MEYLOGENOUS_ | | | | | <ahref=‘http://www.ncbi.nlm.nih. | |
| LEUKEMIA _UP | | | | | gov/gene/947’>[GeneID=947]</a> | |
| | | | | | cells isolated from bone marrow | |
| | | | | | of CML (chronic myelogenous | |
| | | | | | leukemia) patients, compared | |
| | | | | | to those from normal donors. | |
| MORF_RAN | 33 | 268 | 0.123134 | 9.76E−41 | Neighborhood of RAN | A |
| HSIAO_HOUSEKEEP- | 36 | 389 | 0.092545 | 7.98E−40 | Housekeeping genes identified | A |
| ING_GENES | | | | | as expressed across 19 normal | |
| | | | | | tissues. | |
| MORF_UBE2I | 31 | 238 | 0.130252 | 4.38E−39 | Neighborhood of UBE2I | A |
| MORF_RAD23A | 34 | 345 | 0.098551 | 1.43E−38 | Neighborhood of RAD23A | D |
| BLALOCK_ | 49 | 1237 | 0.039612 | 1.49E−36 | Genes down-regulated in brain | C |
| ALZHEIMERS_ | | | | | from patients with Alzheimer's | |
| DISEASE_DN | | | | | disease. | |
| MORF_HDAC1 | 30 | 254 | 0.11811 | 1.63E−36 | Neighborhood of HDAC1 | B |
| DANG_BOUND_BY_ | 46 | 1102 | 0.041742 | 2.89E−35 | Genes whose promoters are | A |
| MYC | | | | | bound by MYC | |
| | | | | | <ahref=‘http://www. | |
| | | | | | ncbi.nlm.nih.gov/gene/4609’> | |
| | | | | | [GeneID=4609]</a>, according | |
| | | | | | to MYC Target Gene Database. | |
| INTRACELLULAR_ | 47 | 1192 | 0.03943 | 6.12E−35 | Genes annotated by the GO | D |
| ORGANELLE _PART | | | | | term GO:0044446. A constituent | |
| | | | | | part of an intracellular organelle, | |
| | | | | | an organized structure of | |
| | | | | | distinctive morphology and | |
| | | | | | function, occurring within the cell. | |
| | | | | | Includes constituent parts of the | |
| | | | | | nucleus, mitochondria, plastids, | |
| | | | | | vacuoles, vesicles, ribosomes and | |
| | | | | | the cytoskeleton but excludes the | |
| | | | | | plasma membrane. | |
| ORGANELLE_PART | 47 | 1197 | 0.039265 | 7.38E−35 | Genes annotated by the GO | D |
| | | | | | term GO:0044422. Any constituent | |
| | | | | | part of an organelle, an organized | |
| | | | | | Structure of distinctive morphology | |
| | | | | | and function. Includes constituent | |
| | | | | | parts of the nucleus, mitochondria, | |
| | | | | | plastids, vacuoles, vesicles, | |
| | | | | | ribosomes and the cytoskeleton, but | |
| | | | | | excludes the plasma membrane. | |
| MORF_ANP32B | 27 | 197 | 0.137056 | 9.45E−35 | Neighborhood of ANP32B | A |
| MORF_DEK | 29 | 262 | 0.110687 | 1.79E−34 | Neighborhood of DEK | B |
| MORF_GNB1 | 30 | 303 | 0.09901 | 3.76E−34 | Neighborhood of GNB1 | D |
| PUJANA_CHEK2_ | 39 | 778 | 0.050129 | 7.75E−33 | Genes constituting the CHEK2- | D |
| PCC _ NETWORK | | | | | PCC network of transcripts whose | |
| | | | | | expression positively correlates | |
| | | | | | (Pearson correlation coefficient, | |
| | | | | | PCC ≧ 0.4) with that of CHEK2 | |
| | | | | | <ahref=‘http://www.ncbi.nlm.nih. | |
| | | | | | gov/gene/11200’>[GeneID= | |
| | | | | | 11200]</a>. | |
| GRADE_COLON_ | 40 | 870 | 0.045977 | 3.12E−32 | Up-regulated genes in colon | A |
| CANCER_UP | | | | | carcinoma tumors compared to the | |
| | | | | | matched normal mucosa samples. | |
| MORF_CTBP1 | 24 | 168 | 0.142857 | 1.94E−31 | Neighborhood of CTBP1 | D |
| MORF_XRCC5 | 26 | 234 | 0.111111 | 4.97E−31 | Neighborhood of XRCC5 | D |
| MORF_BUB3 | 27 | 276 | 0.097826 | 1.16E−30 | Neighborhood of BUB3 | D |
| MORF_CSNK2B | 27 | 287 | 0.094077 | 3.38E−30 | Neighborhood of CSNK2B | D |
| MORF_G22P1 | 23 | 171 | 0.134503 | 1.55E−29 | Neighborhood of G22P1 | D |
| MORF_EIF3S2 | 25 | 244 | 0.102459 | 5.75E−29 | Neighborhood of EIF3S2 | D |
| REACTOME_CELL_ | 29 | 412 | 0.070388 | 1.03E−28 | Genes involved in Cell Cycle | D |
| CYCLE | | | | | | |
| BENPORATH_MYC_ | 35 | 775 | 0.045161 | 5.78E−28 | Set ‘Myc targets2’: targets of c-Myc | D |
| MAX_TARGETS | | | | | <ahref=‘http://www.ncbi.nlm.nih. | |
| | | | | | gov/gene/4609’>[GeneID=4609] | |
| | | | | | </a> and Max <ahref =‘http://www. | |
| | | | | | ncbi.nlm.nih.gov/gene/4149’> | |
| | | | | | [GeneID=4149]</a> identified by | |
| | | | | | Chip on chip in a Burkitt's | |
| | | | | | lymphoma cell line; overlap set. | |
| LOPEZ_MBD_ | 37 | 957 | 0.038662 | 3.57E−27 | Genes up-regulated in HeLa | D |
| TARGETS | | | | | cells (cervical cancer) after | |
| | | | | | simultaneus knockdown of all | |
| | | | | | three MBD (methyl-CpG binding | |
| | | | | | domain) proteins MeCP2, MBD1 | |
| | | | | | and MBD2 [GeneID=4204;4152; | |
| | | | | | 8932] by RNAi. | |
| CYTOPLASM | 50 | 2130 | 0.023474 | 8.02E−27 | Genes annotated by the GO | A |
| | | | | | term GO:0005737. All of the | |
| | | | | | contents of a cell excluding the | |
| | | | | | plasma membrane and nucleus, | |
| | | | | | but including other subcellular | |
| | | | | | structures. | |
| MORF_SKP1A | 22 | 202 | 0.108911 | 3.33E−26 | Neighborhood of SKP1A | D |
| MORF_AATF | 22 | 205 | 0.107317 | 4.64E−26 | Neighborhood of AATF | D |
| MORF_ACP1 | 22 | 210 | 0.104762 | 7.99E−26 | Neighborhood of ACP1 | D |
| REACTOME_PACKAG- | 15 | 48 | 0.3125 | 1.08E−25 | Genes involved in Packaging | A |
| ING_OF_TELOMERE_ | | | | | Of Telomere Ends | |
| ENDS | | | | | | |
| PENG_GLUTAMINE_ | 25 | 337 | 0.074184 | 1.97E−25 | Genes down-regulated in BJAB | D |
| DEPRIVATION_DN | | | | | cells (B-lymphoma) after | |
| | | | | | glutamine <ahref=‘http://pubchem. | |
| | | | | | ncbi.nlm.nih.gov/summary/ | |
| | | | | | summary.cgi?cid=738’> | |
| | | | | | [PubChem=738]</a> deprivation. | |
| PENG_LEUCINE_ | 21 | 187 | 0.112299 | 2.39E−25 | Genes down-regulated in BJAB | D |
| DEPRIVATION_DN | | | | | cells (B-lymphoma) after leucine | |
| | | | | | <ahref=‘http://pubchem.ncbi.nlm. | |
| | | | | | nih.gov/summary/summary. cgi? | |
| | | | | | cid=857’>[PubChem=857]</a> | |
| | | | | | deprivation. | |
| CASORELLI_ACUTE_ | 31 | 662 | 0.046828 | 2.71E−25 | Genes down-regulated in APL | D |
| PROMYELOCYTIC_ | | | | | (acute promyeolocytic leukemia) | |
| LEUKEMIA_ DN | | | | | blasts expressing PML-RARA | |
| | | | | | fusion [GeneID=5371;5914] | |
| | | | | | compared to normal promyeloblasts. | |
| MORF_DAP3 | 21 | 193 | 0.108808 | 4.73E−25 | Neighborhood of DAP3 | D |
| MORF_NPM1 | 20 | 162 | 0.123457 | 4.83E−25 | Neighborhood of NPM1 | A |
| PUJANA_ATM_PCC_ | 41 | 1440 | 0.028472 | 4.84E−25 | Genes constituting the ATM-PCC | D |
| N ETWORK | | | | | network of transcripts whose | |
| | | | | | expression positively correlated | |
| | | | | | (Pearson correlation coefficient, | |
| | | | | | PCC ≧ 0.4) with that of ATM | |
| | | | | | <ahref=‘http://www.ncbi.nlm. | |
| | | | | | nih.gov/gene/472’>[GeneID=472] | |
| | | | | | </a> across a compendium of | |
| | | | | | normal tissues. | |
| KIM_BIPOLAR_ | 31 | 681 | 0.045521 | 6.27E−25 | Genes whose expression | C |
| DISORDER_OLIGO- | | | | | significantly and positively | |
| DENDROCYTE_ | | | | | correlated with oligodendrocyte | |
| DENSITY_CORR_UP | | | | | density in layer VI of BA9 brain | |
| | | | | | region in patients with bipolar | |
| | | | | | disorder. | |
| REACTOME_MEIOTIC_ | 16 | 71 | 0.225352 | 9.73E−25 | Genes involved in Meiotic | A |
| SYNAPSIS | | | | | Synapsis | |
| MORF_SOD1 | 23 | 277 | 0.083032 | 1.34E−24 | Neighborhood of SOD1 | D |
| GRAESSMANN_ | 44 | 1781 | 0.024705 | 1.99E−24 | Genes down-regulated in ME- | D |
| APOPTOSIS_BY_ | | | | | A cells (breast cancer) undergoing | |
| DOXORUBICIN_DN | | | | | apoptosis in response to doxorubi- | |
| | | | | | cin <ahref=‘http://pubchem.ncbi. | |
| | | | | | nlm.nih.gov/summary/summary. | |
| | | | | | cgi?cid= 31703’>[PubChem= | |
| | | | | | 31703]</a>. | |
| NUCLEUS | 40 | 1428 | 0.028011 | 3.57E−24 | Genes annotated by the GO term | D |
| | | | | | GO:0005634. A membrane- | |
| | | | | | bounded organelle of eukaryotic | |
| | | | | | cells in which chromosomes are | |
| | | | | | housed and replicated. In most cells, | |
| | | | | | the nucleus contains all of the | |
| | | | | | cell's chromosomes except the | |
| | | | | | organellar chromosomes, and is | |
| | | | | | the site of RNA synthesis and | |
| | | | | | processing. In some species, or in | |
| | | | | | specialized cell types, RNA | |
| | | | | | metabolism or DNA replication | |
| | | | | | may be absent. | |
| MACROMOLECULAR_ | 34 | 945 | 0.035979 | 5.28E−24 | Genes annotated by the GO term | D |
| COMPLEX | | | | | GO:0032991. A stable assembly of | |
| | | | | | two or more macromolecules, i.e. | |
| | | | | | proteins, nucleic acids, carbohy- | |
| | | | | | drates or lipids, in which the | |
| | | | | | constituent parts function together. | |
| MORF_RAD21 | 20 | 182 | 0.10989 | 5.34E−24 | Neighborhood of RAD21 | D |
| REACTOME_RNA_POL_ | 15 | 61 | 0.245902 | 6.69E−24 | Genes involved in RNA | A |
| I_PROM_OTER_OPENING | | | | | Polymerase I Promoter Opening | |
| DODD_NASOPHARYN- | 39 | 1368 | 0.028509 | 7.61E−24 | Genes down-regulated in | D |
| GEAL_CARCINOMA_ | | | | | nasopharyngeal carcinoma (NPC) | |
| DN | | | | | compared to the normal tissue. | |
| MORF_NME2 | 19 | 154 | 0.123377 | 7.7E−24 | Neighborhood of NME2 | A |
| MORF_PPP2CA | 18 | 127 | 0.141732 | 8.9E−24 | Neighborhood of PPP2CA | D |
| PILON_KLF1_ | 45 | 1972 | 0.022819 | 1.33E−23 | Genes down-regulated in | D |
| TARGETS_DN | | | | | erythroid progenitor cells from | |
| | | | | | fetal livers of E13.5 embryos with | |
| | | | | | KLF1 | |
| | | | | | <ahref=‘http://www.ncbi.nlm. | |
| | | | | | nih.gov/gene/10661’>[GeneID= | |
| | | | | | 10661]</a> knockout compared to | |
| | | | | | those from the wild type embryos. | |
| REACTOME_DEPOSI- | 15 | 64 | 0.234375 | 1.5E−23 | Genes involved in Deposition of | A |
| TION_OF_NEW_ | | | | | New CENPA-containing | |
| CENPA_CONTAINING_ | | | | | Nucleosomes at the Centromere | |
| NUCLEOSOMES_AT_ | | | | | | |
| THE_CENTROMERE | | | | | | |
| GGGCGGR_V$SP1_Q6 | 53 | 2939 | 0.018033 | 3.63E−23 | Genes with promoter regions [−2kb, | D |
| | | | | | 2kb] around transcription start site | |
| | | | | | containing the motif GGGCGGR | |
| | | | | | which matches annotation for | |
| | | | | | SP1:Sp1 transcription factor | |

Table 4 below shows, by letter coding, the relatedness of the pathways for the drugs (Compound B, Tubastatin A, and Compound A) tested in the pathway analysis.

TABLE 4

| | | Code |
|---|---|---|
| in Compound B & Tubastatin A & Compound A | 14 | A |
| in Compound B & Tubastatin A but not Compound A | 2 | B |
| in Compound B & Compound A but not Tubastatin A | 2 | C |
| in Compound B only | 32 | D |

TABLE 5
(Tubastatin A (+))

| Pathway | Observed | Pathway_size | Ratio | P-value | Description | Code |
|---|---|---|---|---|---|---|
| HSIAO_HOUSE- | 68 | 389 | 0.174807 | 1.32606E−81 | Housekeeping genes identified | A |
| KEEPING_GENES | | | | | as expressed across 19 normal | |
| | | | | | tissues. | |
| REACTOME_RNA_ | 40 | 61 | 0.655738 | 2.55683E−76 | Genes involved in RNA | A |
| POL_I_PROMOTER_ | | | | | Polymerase I Promoter | |
| OPENING | | | | | Opening | |
| KEGG_SYSTEMIC_ | 47 | 139 | 0.338129 | 2.31143E−71 | Systemic lupus erythematosus | E |
| LUPUS_ERYTHEMA- | | | | | | |
| TOSUS | | | | | | |
| REACTOME_ | 41 | 84 | 0.488095 | 1.47655E−70 | Genes involved in Meiotic | E |
| MEIOTIC_RECOM- | | | | | Recombination | |
| BINATION | | | | | | |
| REACTOME_AMY- | 40 | 81 | 0.493827 | 4.05384E−69 | Genes involved in Amyloids | E |
| LOIDS | | | | | | |
| REACTOME_RNA_ | 40 | 86 | 0.465116 | 1.00565E−67 | Genes involved in RNA | E |
| POL_I_TRANSCRIP- | | | | | PolymeraselTranscription | |
| TION | | | | | | |
| REACTOME_MEIOSIS | 42 | 112 | 0.375 | 4.41037E−66 | Genes involved in Meiosis | E |
| REACTOME_RNA_ | 40 | 119 | 0.336134 | 1.21576E−60 | Genes involved in RNA | E |
| POL_I_RNA_POL_ | | | | | Polymerase I, RNA | |
| III_AND_MITOCHON- | | | | | Polymerase III, and | |
| DRIAL_TRANSCRIP- | | | | | Mitochondrial Transcription | |
| TION | | | | | | |
| MORF_NPM1 | 43 | 162 | 0.265432 | 5.15E−60 | Neighborhood of NPM1 | A |
| REACTOME_TRANS- | 45 | 207 | 0.217391 | 2.0126E−58 | Genes involved in | E |
| CRIPTION | | | | | Transcription | |
| MODULE_83 | 50 | 320 | 0.15625 | 3.51799E−57 | Genes in the cancer module 83 | E |
| PUJANA_BRCA1_ | 84 | 1651 | 0.050878 | 4.37079E−56 | Genes constituting the | A |
| PCC _NETWORK | | | | | BRCA1-PCC network of | |
| | | | | | transcripts whose expression | |
| | | | | | positively correlated (Pearson | |
| | | | | | correlation coefficient, PCC ≧ | |
| | | | | | 0.4) with That of BRCA1 | |
| | | | | | <ahref=‘http://www.ncbi.nlm. | |
| | | | | | nih.gov/gene/672’>[GeneID= | |
| | | | | | 672]</a> across a compendium | |
| | | | | | of normal tissues. | |
| REACTOME_METABO- | 56 | 496 | 0.112903 | 6.93223E−56 | Genes involved in Metabolism | E |
| LISM_OF_PROTEINS | | | | | of proteins | |
| MODULE_151 | 49 | 318 | 0.154088 | 1.01135E−55 | Genes in the cancer module 151 | E |
| MORF_ACTG1 | 39 | 138 | 0.282609 | 1.09872E−55 | Neighborhood of ACTG1 | E |
| MORF_RAN | 46 | 268 | 0.171642 | 1.34399E−54 | Neighborhood of RAN | A |
| MODULE_114 | 49 | 338 | 0.14497 | 2.33475E−54 | Genes in the cancer module 114 | E |
| MORF_NME2 | 39 | 154 | 0.253247 | 1.45001E−53 | Neighborhood of NME2 | A |
| GCM_NPM1 | 36 | 116 | 0.310345 | 3.77137E−53 | Neighborhood of NPM1 | E |
| MORF_UBE2I | 43 | 238 | 0.180672 | 4.45899E−52 | Neighborhood of UBE2I | A |
| KEGG_RIBOSOME | 33 | 88 | 0.375 | 4.86486E−52 | Ribosome | E |
| MARTENS_TRETI- | 63 | 841 | 0.074911 | 9.41755E−52 | Genes down-regulated in NB4 | E |
| NOIN_RESPONSE_DN | | | | | cells (acute promyelocytic | |
| | | | | | leukemia, APL) in response to | |
| | | | | | tretinoin <ahref=‘http://pubchem. | |
| | | | | | ncbi.nlm.nih.gov/summary/ | |
| | | | | | summary.cgi?cid=5538’>[Pub | |
| | | | | | Chem=5538]</a>; based on | |
| | | | | | Chip-seq data. | |
| REACTOME_SRP_ | 39 | 170 | 0.229412 | 1.08908E−51 | Genes involved in SRP- | E |
| DEPENDENT _CO- | | | | | dependent cotranslational | |
| TRANSLATIONAL_ | | | | | protein targeting to membrane | |
| PROTEIN_ | | | | | | |
| TARGETING_TO_ | | | | | | |
| MEMBRANE | | | | | | |
| MIPS_RIBOSOME_ | 32 | 81 | 0.395062 | 2.19874E−51 | Ribosome, cytoplasmic | E |
| CYTOPLASMIC | | | | | | |
| DANG_BOUND_BY_ | 68 | 1102 | 0.061706 | 2.10605E−50 | Genes whose promoters are | A |
| MYC | | | | | bound by MYC <ahref=‘http:// | |
| | | | | | www.ncbi.nlm.nih.gov/gene/ | |
| | | | | | 4609’>[GeneID=4609]</a>, | |
| | | | | | according to MYC Target Gene | |
| | | | | | Database. | |
| GRADE_COLON_ | 62 | 870 | 0.071264 | 1.332E−49 | Up-regulated genes in colon | A |
| CANCER_UP | | | | | carcinoma tumors compared | |
| | | | | | to the matched normal mucosa | |
| | | | | | samples. | |
| MIPS_NOP56P_ | 33 | 104 | 0.317308 | 3.72844E−49 | Nop56p-associated pre-rRNA | E |
| ASSOCIATED_PRE_ | | | | | complex | |
| RRNA_COMPLEX | | | | | | |
| GCM_ACTG1 | 34 | 124 | 0.274194 | 4.09074E−48 | Neighborhood of ACTG1 | E |
| REACTOME_TRANS- | 39 | 211 | 0.184834 | 1.09221E−47 | Genes involved in Translation | E |
| LATION | | | | | | |
| MORF_TPT1 | 32 | 102 | 0.313725 | 1.67005E−47 | Neighborhood of TPT1 | E |
| MORF_ANP32B | 38 | 197 | 0.192893 | 3.0616E−47 | Neighborhood of ANP32B | A |
| GNF2_EIF3S6 | 33 | 121 | 0.272727 | 1.23617E−46 | Neighborhood of ElF3S6 | E |
| REACTOME_PACK- | 26 | 48 | 0.541667 | 1.26746E−46 | Genes involved in Packaging | A |
| AGING_OF_TELO- | | | | | Of Telomere Ends | |
| MERE_ENDS | | | | | | |
| CYTOPLASM | 83 | 2130 | 0.038967 | 1.91899E−46 | Genes annotated by the GO | A |
| | | | | | term GO:0005737. All of the | |
| | | | | | contents of a cell excluding | |
| | | | | | the plasma membrane and | |
| | | | | | nucleus, but including other | |
| | | | | | subcellular structures. | |
| REACTOME_INFLU- | 37 | 195 | 0.189744 | 9.97519E−46 | Genes involved in Influenza | E |
| ENZA_LIFE_CYCLE | | | | | Life Cycle | |
| GCM_TPT1 | 28 | 70 | 0.4 | 2.92888E−45 | Neighborhood of TPT1 | E |
| MOOTHA_HUM | 46 | 429 | 0.107226 | 7.82687E−45 | Mitochondrial genes; based | E |
| AN_MITODB_6_2002 | | | | | on literature and sequence | |
| | | | | | annotation resources and | |
| | | | | | converted to Affymetrix HG- | |
| | | | | | U133A probe sets. | |
| REACTOME_DEPOSI- | 27 | 64 | 0.421875 | 1.8209E−44 | Genes involved in Deposition | A |
| TION_OF_NEW_ | | | | | of New CENPA-containing | |
| CENPA_CONTAINING_ | | | | | Nucleosomes at the Centromere | |
| NUCLEOSOM ES_AT_ | | | | | | |
| THE_CENTROMERE | | | | | | |
| MORF_DEK | 39 | 262 | 0.148855 | 8.56568E−44 | Neighborhood of DEK | B |
| STRUCTURAL_ | 28 | 80 | 0.35 | 2.89995E−43 | Genes annotated by the GO | E |
| CONSTITUENT_OF_ | | | | | term GO:0003735. The action | |
| RIBOSOME | | | | | of a molecule that contributes | |
| | | | | | to the structural integrity of the | |
| | | | | | ribosome. | |
| CYTOPLASMIC_PART | 67 | 1383 | 0.048445 | 5.72269E−43 | Genes annotated by the GO | E |
| | | | | | term GO:0044444. Any | |
| | | | | | constituent part of the cytoplasm, | |
| | | | | | all of the contents of a cell | |
| | | | | | excluding the plasma membrane | |
| | | | | | and nucleus, but including | |
| | | | | | other subcellular structures. | |
| REACTOME_MEI | 27 | 71 | 0.380282 | 6.10173E−43 | Genes involved in Meiotic | A |
| OTIC_SYNAPSIS | | | | | Synapsis | |
| REACTOME_INFLU- | 33 | 161 | 0.204969 | 4.5827E−42 | Genes involved in Influenza | E |
| ENZA_VIRAL_RNA_ | | | | | Viral RNA Transcription and | |
| TRANSCRIPTION_ | | | | | Replication | |
| AND_REPLCATION | | | | | | |
| REACTOME_PEPTIDE_ | 32 | 145 | 0.22069 | 6.07377E−42 | Genes involved in Peptide | E |
| CHAIN_ELONGATION | | | | | chain elongation | |
| GNF2_DAP3 | 30 | 119 | 0.252101 | 2.60612E−41 | Neighborhood of DAP3 | F |
| MORF_HDAC1 | 37 | 254 | 0.145669 | 3.2396E−41 | Neighborhood of HDAC1 | B |
| MODULE_32 | 36 | 241 | 0.149378 | 1.56382E−40 | Genes in the cancer module 32 | F |
| REACTOME_TELO- | 26 | 75 | 0.346667 | 4.08693E−40 | Genes involved in Telomere | E |
| MERE_MAINTENANCE | | | | | Maintenance | |
| REACTOME_3_UTR_ | 32 | 166 | 0.192771 | 6.8601E−40 | Genes involved in 3'-UTR- | E |
| MEDIATED_TRANS- | | | | | mediated translational | |
| LATIONAL_REGULA- | | | | | regulation | |
| TION | | | | | | |
| REACTOME_NON- | 32 | 167 | 0.191617 | 8.44451E−40 | Genes involved in Nonsense | E |
| SENSE_MEDIATED_ | | | | | Mediated Decay Enhanced by | |
| DECAY_ENHANCED_ | | | | | the Exon Junction Complex | |
| BY_THE_EXON_ | | | | | | |
| JUNCTION_COMPLEX | | | | | | |

Table 6 below shows, by letter coding, the relatedness of the pathways for the drugs (Compound B, Tubastatin A, and Compound A) tested in the pathway analysis.

TABLE 6

| | | Code |
|---|---|---|
| in Compound B & Tubastatin A & Compound A | 14 | A |
| in Compound B & Tubastatin A but not Compound A | 2 | B |
| in Tubastatin A & Compound A but not Compound B | 32 | E |
| in Tubastatin A only | 2 | F |

TABLE 7
(Compound A(+))

| Pathway | Observed | Pathway_size | Ratio | P-value | Description | Code |
|---|---|---|---|---|---|---|
| HSIAO_HOUSEKEEP- | 87 | 389 | 0.22365 | 0 | Housekeeping genes identified | A |
| ING_GENES | | | | | as expressed across 19 normal | |
| | | | | | tissues. | |
| REACTOME_RNA_ | 49 | 61 | 0.803279 | 2.77678E−95 | Genes involved in RNA | A |
| POL_I_PROMOTER_ | | | | | Polymerase I Promoter | |
| OPENING | | | | | Opening | |
| REACTOME_MEIOTIC_ | 50 | 84 | 0.595238 | 3.17678E−86 | Genes involved in Meiotic | E |
| RECOMBINATION | | | | | Recombination | |
| REACTOME_RNA_ | 50 | 86 | 0.581395 | 1.82036E−85 | Genes involved in RNA | E |
| POL_I_TRANSCRIP- | | | | | Polymerase I Transcription | |
| TION | | | | | | |
| REACTOME_ | 49 | 81 | 0.604938 | 5.09913E−85 | Genes involved in Amyloids | E |
| AMYLOIDS | | | | | | |
| MORF_NPM1 | 59 | 162 | 0.364198 | 5.95145E−85 | Neighborhood of NPM1 | A |
| KEGG_SYSTEMIC_ | 54 | 139 | 0.388489 | 1.32834E−79 | Systemic lupus erythematosus | E |
| LUPUS_ERYTHE- | | | | | | |
| MATOSUS | | | | | | |
| MORF_ACTG1 | 53 | 138 | 0.384058 | 8.42596E−78 | Neighborhood of ACTG1 | E |
| REACTOME_MEIOSIS | 50 | 112 | 0.446429 | 1.49288E−77 | Genes involved in Meiosis | E |
| REACTOME_RNA_ | 50 | 119 | 0.420168 | 7.42228E−76 | Genes involved in RNA | E |
| POL_I_RNA_POL_III_ | | | | | Polymerase I, RNA | |
| AND_MITOCHONDRI- | | | | | Polymerase III, and | |
| AL_TRANSCRIPTION | | | | | Mitochondrial Transcription | |
| MODULE_83 | 65 | 320 | 0.203125 | 9.3936E−75 | Genes in the cancer module 83 | E |
| MODULE_114 | 64 | 338 | 0.189349 | 1.74872E−71 | Genes in the cancer module 114 | E |
| PUJANA_BRCA1_ | 107 | 1651 | 0.064809 | 1.33441E−70 | Genes constituting the | A |
| PCC _NETWORK | | | | | BRCA1-PCC network of | |
| | | | | | transcripts whose expression | |
| | | | | | positively correlated (Pearson | |
| | | | | | correlation coefficient, PCC ≧ | |
| | | | | | 0.4) with that of BRCA1<ahref= | |
| | | | | | ‘http://www.ncbi.nlm.nih.gov/ | |
| | | | | | gene/672’>[GeneID=672]</a> | |
| | | | | | across a compendium of normal | |
| | | | | | tissues. | |
| CYTOPLASM | 117 | 2130 | 0.05493 | 9.53592E−70 | Genes annotated by the GO | A |
| | | | | | term GO:0005737. All of the | |
| | | | | | contents of a cell excluding the | |
| | | | | | plasma membrane and nucleus, | |
| | | | | | but including other subcellular | |
| | | | | | structures. | |
| MIPS_RIBOSOME_ | 42 | 81 | 0.518519 | 7.22853E−69 | Ribosome, cytoplasmic | E |
| CYTOPLASMIC | | | | | | |
| MODULE_151 | 61 | 318 | 0.191824 | 1.76308E−68 | Genes in the cancer module 151 | E |
| MOOTHA_HUMAN_ | 66 | 429 | 0.153846 | 1.98815E−67 | Mitochondrial genes; based on | E |
| MITODB_6_2002 | | | | | literature and sequence annota- | |
| | | | | | tion resources and converted to | |
| | | | | | Affymetrix HG-U133A probe | |
| | | | | | sets. | |
| REACTOME_METAB- | 69 | 496 | 0.139113 | 2.15041E−67 | Genes involved in Metabolism | E |
| OLISM_OF_PROTEINS | | | | | of proteins | |
| MIPS_NOP56P_ | 44 | 104 | 0.423077 | 5.79581E−67 | Nop56p-associated pre-rRNA | E |
| ASSOCIATED_PRE_ | | | | | complex | |
| RRNA_COMPLEX | | | | | | |
| KEGG_RIBOSOME | 42 | 88 | 0.477273 | 8.20158E−67 | Ribosome | E |
| REACTOME_TRANS- | 53 | 207 | 0.256039 | 8.42324E−67 | Genes involved in Transcription | E |
| CRIPTION | | | | | | |
| MORF_UBE2I | 55 | 238 | 0.231092 | 1.38992E−66 | Neighborhood of UBE2I | A |
| CYTOPLASMIC_PART | 96 | 1383 | 0.069414 | 1.10511E−65 | Genes annotated by the GO term | E |
| | | | | | GO:0044444. Any constituent | |
| | | | | | part of the cytoplasm, all of the | |
| | | | | | contents of a cell excluding the | |
| | | | | | plasma membrane and nucleus, | |
| | | | | | but including other subcellular | |
| | | | | | structures. | |
| REACTOME_PACK- | 35 | 48 | 0.729167 | 1.88442E−65 | Genes involved in Packaging | A |
| AGING_OF_TELO- | | | | | Of Telomere Ends | |
| MERE_ENDS | | | | | | |
| MORF_NME2 | 48 | 154 | 0.311688 | 3.10557E−65 | Neighborhood of NME2 | A |
| DANG_BOUND_BY_ | 88 | 1102 | 0.079855 | 3.8618E−65 | Genes whose promoters are | A |
| MYC | | | | | bound by MYC <ahref=‘http:// | |
| | | | | | www.ncbi.nlm.nih.gov/gene/ | |
| | | | | | 4609’>[GeneID=4609]</a>, | |
| | | | | | according to MYC Target Gene | |
| | | | | | Database. | |
| GCM_TPT1 | 37 | 70 | 0.528571 | 3.55595E−61 | Neighborhood of TPT1 | E |
| MORF_RAN | 53 | 268 | 0.197761 | 3.04985E−60 | Neighborhood of RAN | A |
| REACTOME_SRP_ | 46 | 170 | 0.270588 | 2.87124E−59 | Genes involved in SRP- | E |
| DEPENDENT _CO- | | | | | dependent cotranslational | |
| TRANSLATIONAL_ | | | | | protein targeting to membrane | |
| PROTEIN_TARGET- | | | | | | |
| ING_TO_MEMBRANE | | | | | | |
| MORF_TPT1 | 40 | 102 | 0.392157 | 2.87425E−59 | Neighborhood of TPT1 | E |
| GCM_NPM1 | 41 | 116 | 0.353448 | 1.83552E−58 | Neighborhood of NPM1 | E |
| REACTOME_INFLU | 47 | 195 | 0.241026 | 6.91736E−58 | Genes involved in Influenza | E |
| ENZA_LIFE_CYCLE | | | | | Life Cycle | |
| REACTOME_TRANS- | 48 | 211 | 0.227488 | 8.99426E−58 | Genes involved in Translation | E |
| LATION | | | | | | |
| GRADE_COLON_ | 75 | 870 | 0.086207 | 1.10441E−57 | Up-regulated genes in colon | A |
| CANCER_UP | | | | | carcinoma tumors compared to | |
| | | | | | the matched normal mucosa | |
| | | | | | samples. | |
| GNF2_EIF3S6 | 41 | 121 | 0.338843 | 1.47035E−57 | Neighborhood of EIF3S6 | E |
| REACTOME_TELO- | 36 | 75 | 0.48 | 1.73836E−57 | Genes involved in Telomere | E |
| MERE_MAINTE- | | | | | Maintenance | |
| NANCE | | | | | | |
| REACTOME_MEIO- | 35 | 71 | 0.492958 | 1.86284E−56 | Genes involved in Meiotic | A |
| TIC_SYNAPSIS | | | | | Synapsis | |
| REACTOME_DEPOSI- | 34 | 64 | 0.53125 | 2.19681E−56 | Genes involved in Deposition | A |
| TION_OF_NEW_ | | | | | of New CENPA-containing | |
| CENPA_CONTAIN- | | | | | Nucleosomes at the Centromere | |
| ING_NUCLEOSOMES_ | | | | | | |
| AT_THE_CENTRO- | | | | | | |
| MERE | | | | | | |
| REACTOME_3_UTR_ | 43 | 166 | 0.259036 | 1.62802E−54 | Genes involved in 3′-UTR- | E |
| MEDIATED_TRANS- | | | | | mediated translational | |
| LATIONAL_REGULA- | | | | | regulation | |
| TION | | | | | | |
| REACTOME_PEPTIDE_ | 41 | 145 | 0.282759 | 8.72138E−54 | Genes involved in Peptide | E |
| CHAIN_ELONGATION | | | | | chain elongation | |
| REACTOME_INFLU- | 42 | 161 | 0.26087 | 2.08571E−53 | Genes involved in Influenza | E |
| ENZA_VIRAL_RNA_ | | | | | Viral RNA Transcription and | |
| TRANSCRIPTION_ | | | | | Replication | |
| AND_REPLICATION | | | | | | |
| MOOTHA_MITO- | 57 | 447 | 0.127517 | 2.18748E−53 | Mitochondrial genes | G |
| CHONDRIA | | | | | | |
| GCM_ACTG1 | 39 | 124 | 0.314516 | 2.72657E−53 | Neighborhood of ACTG1 | E |
| BLALOCK_ALZHEI- | 81 | 1237 | 0.065481 | 4.17496E−53 | Genes down-regulated in brain | C |
| MERS_DISEASE_DN | | | | | from patients with Alzheimer's | |
| | | | | | disease. | |
| MARTENS_TRETI- | 69 | 841 | 0.082045 | 1.51166E−51 | Genes down-regulated in NB4 | E |
| NOIN_RESPONSE_DN | | | | | cells (acute promyelocytic | |
| | | | | | leukemia, APL) in response to | |
| | | | | | tretinoin <ahref=‘http://pubchem. | |
| | | | | | ncbi.nlm.nih.gov/summary/ | |
| | | | | | summary.cgi?cid=5538’>[Pub | |
| | | | | | Chem=5538]</a>; based on | |
| | | | | | Chip-seq data. | |
| KIM_BIPOLAR_ | 64 | 681 | 0.093979 | 1.83662E−51 | Genes whose expression | C |
| DISORDER_OLIG- | | | | | significantly and positively | |
| ODENDROCYTE_ | | | | | correlated with oligodendrocyte | |
| DENSITY_CORR_UP | | | | | density in layer VI of BA9 brain | |
| | | | | | region in patients with bipolar | |
| | | | | | disorder. | |
| KRCTCNNNNMA- | 32 | 66 | 0.484848 | 2.20314E−51 | Genes with promoter regions | G |
| NAGC_UNKNOWN | | | | | [−2kb,2kb] around transcription | |
| | | | | | start site containing motif | |
| | | | | | KRCTCNNNNMANAGC. | |
| | | | | | Motif does not match any known | |
| | | | | | transcription factor | |
| MORF_ANP32B | 43 | 197 | 0.218274 | 5.84661E−51 | Neighborhood of ANP32B | A |
| REACTOME_NON- | 41 | 167 | 0.245509 | 6.15053E−51 | Genes involved in Nonsense | E |
| SENSE_MEDIATED_ | | | | | Mediated Decay Enhanced by | |
| DECAY_ENHANCED_ | | | | | the Exon Junction Complex | |
| BY_THE_EXON_ | | | | | | |
| JUNCTION_COMPLEX | | | | | | |
| STRUCTURAL_ | 33 | 80 | 0.4125 | 5.97757E−50 | Genes annotated by the GO | E |
| CONSTITUENT_OF_ | | | | | term GO:0003735. The action | |
| RIBOSOME | | | | | of a molecule that contributes | |
| | | | | | to the structural integrity of the | |
| | | | | | ribosome. | |

Table 8 below shows, by letter coding, the relatedness of the pathways for the drugs (Compound B, Tubastatin A, and Compound A) tested in the pathway analysis.

TABLE 8

| | | Code |
|---|---|---|
| in Compound B & Tubastatin A & Compound A | 14 | A |
| In Compound B & Compound A but not Tubastatin A | 2 | C |
| in Tubastatin A & Compound A but not Compound B | 32 | E |
| in Compound A only | 2 | G |

### Example 4

### Analysis of Total Peptide (Including Both Acetylated and Unacetylated) Amount in Compound B Treated Cell Lysate

The SILAC method was performed as described in Example 1. The Compound B- (¹²C-lysine labeled) and DMSO-treated (¹³C-lysine labeled) cells were lysed with 2% SDS at 97° C. for 5 min. The samples were sonicated using a high intensity ultrasonic processor, and centrifuged at 20,000 g at 4° C. for 15 min to remove remaining debris. Protein content in the supernatant was determined with a BCA assay kit (Thermo Fisher, Waltham, Mass.) according to the manufacturer's instructions. 25 μg of crude protein from each sample was mixed and separated by 12% SDS-PAGE. After electrophoresis, the gel was stained with Coomassie Blue. The entire gel lane was cut into 20 slices and digested with trypsin. The gel bands were cut into small cubes of 1 mm³ that were washed with 500 μl of H₂O followed by 500 μl of 50% ethanol overnight on a mixer. The gel pieces were dehydrated by the addition of 500 μl of acetonitrile. Supernatant was discarded and the gel pieces were vacuum-dried. Disulfide bonds were cleaved by incubating the gels for 1 hr at 56° C. with 200 μl of 10 mM DTT in 50 mM ammonium bicarbonate buffer. Alkylation of cysteines was performed by incubating the gels for 45 min at room temperature in darkness with 200 μl of 55 mM iodoacetamide in 50 mM ammonium bicarbonate buffer. The gel pieces were dehydrated and vacuum-dried again. Gel pieces were covered with trypsin solution (10 ng/μl in 50 mM ammonium bicarbonate buffer). After a 30-min incubation at 4° C., the remaining trypsin solution was removed, and 50 mM ammonium bicarbonate was added to prevent the gels from drying during overnight digestion at 37° C. The peptides in the gels were extracted once by 200 μl 5% TFA in 50% acetonitrile and twice by 200 μl 100% acetonitrile. All the supernatant was combined and vacuum-dried followed by mass spectrometer analysis.

The results of this experiment for Compound B treated cells are shown in Table 9 below. These results show the total peptide (acetylated and unacetylated) amounts in Compound B treated cell lysate. The data shows which peptide levels changed upon treatment with Compound B, regardless of their acetylation status.

The first column of Table 9 shows the name of the gene for the identified peptides.

The second column of Table 9 shows the ratio of light to heavy peptides, after normalization.

The third column of Table 9 shows the median value of the amount of change, which was determined from the ratio of light to heavy peptides, after normalization (the second column). It is important to note that if the median value for the fold of change was less than 1.3, then the fourth column and the fifth column are blank (data is not shown) because the peptides identified were not of interest.

The fourth column of Table 9 shows that the fold of change transitions from a decrease, at the top of the table, to an increase, at the top of the table.

The fifth column of Table 9 shows the peptide sequences of each peptide identified.

Lengthy table referenced here
US20140357512A1-20141204-T00001
Please refer to the end of the specification for access instructions.

### Example 5

### Synthesis of 2-(diphenylamino)-N-(7-(hydroxyamino)-7-oxoheptyl)pyrimidine-5-carboxamide (Compound A)

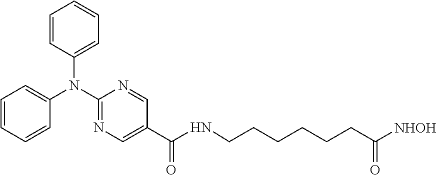

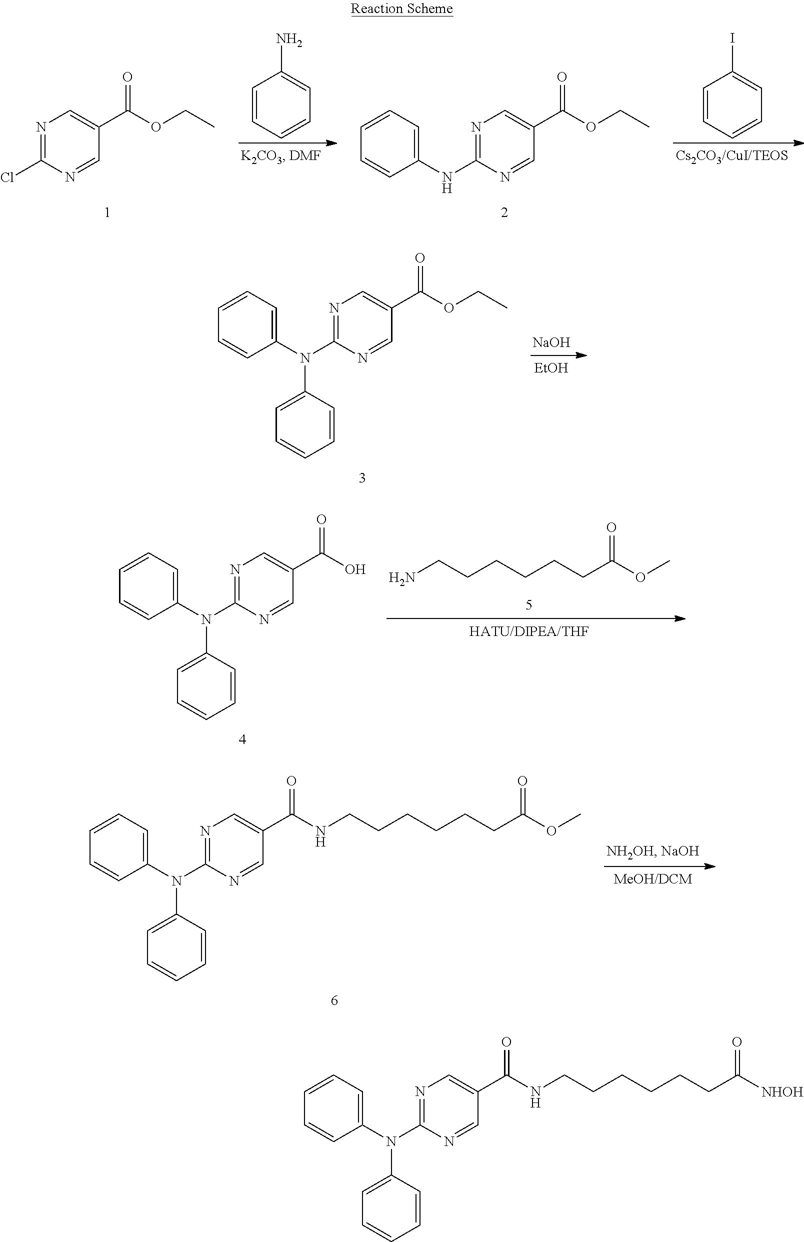

### Synthesis of Intermediate 2

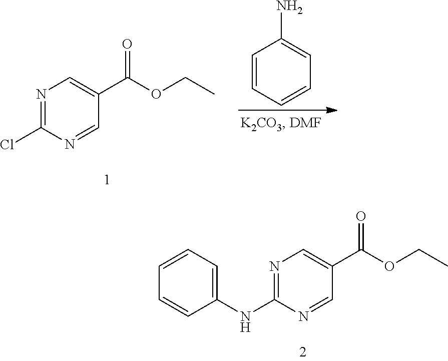

A mixture of aniline (3.7 g, 40 mmol), ethyl 2-chloropyrimidine-5-carboxylate 1 (7.5 g, 40 mmol), K₂CO₃ (11 g, 80 mmol) in DMF (100 ml) was degassed and stirred at 120° C. under N₂ overnight. The reaction mixture was cooled to rt and diluted with EtOAc (200 ml), then washed with saturated brine (200 ml×3). The organic layer was separated and dried over Na₂SO₄, evaporated to dryness and purified by silica gel chromatography (petroleum ethers/EtOAc=10/1) to give the desired product as a white solid (6.2 g, 64%).

### Synthesis of Intermediate 3

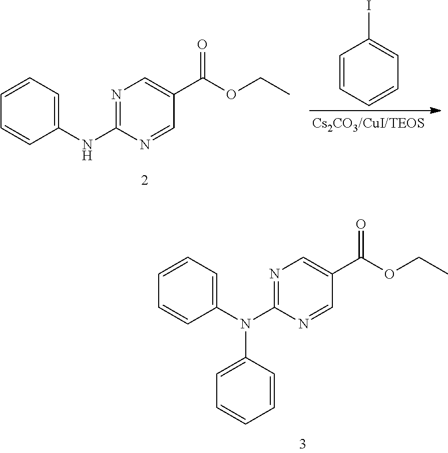 [SMILES: CCOC(=O)c1cnc(Nc2ccccc2)nc1 ; Ic1ccccc1 ; CCOC(=O)c1cnc(N(c2ccccc2)c2ccccc2)nc1]

A mixture of the compound 2 (6.2 g, 25 mmol), iodobenzene (6.12 g, 30 mmol), CuI (955 mg, 5.0 mmol), Cs₂CO₃ (16.3 g, 50 mmol) in TEOS (200 ml) was degassed and purged with nitrogen. The resulting mixture was stirred at 140° C. for 14 h. After cooling to rt, the residue was diluted with EtOAc (200 ml) and 95% EtOH (200 ml), NH₄F—H₂O on silica gel [50 g, pre-prepared by the addition of NH₄F (100 g) in water (1500 ml) to silica gel (500 g, 100-200 mesh)] was added, and the resulting mixture was kept at rt for 2 h, the solidified materials was filtered and washed with EtOAc. The filtrate was evaporated to dryness and the residue was purified by silica gel chromatography (petroleum ethers/EtOAc=10/1) to give a yellow solid (3 g, 38%).

### Synthesis of Intermediate 4

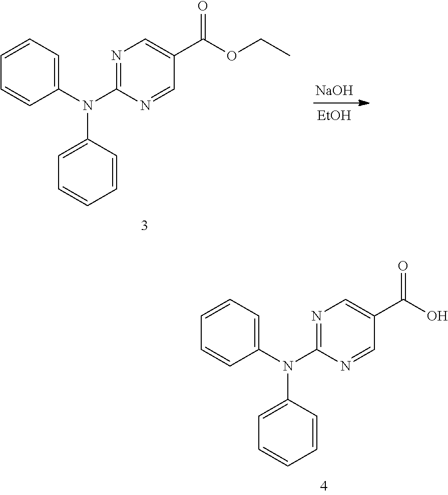

2N NaOH (200 ml) was added to a solution of the compound 3 (3.0 g, 9.4 mmol) in EtOH (200 ml). The mixture was stirred at 60° C. for 30 min. After evaporation of the solvent, the solution was neutralized with 2N HCl to give a white precipitate. The suspension was extracted with EtOAc (2×200 ml), and the organic layer was separated, washed with water (2×100 ml), brine (2×100 ml), and dried over Na₂SO₄. Removal of solvent gave a brown solid (2.5 g, 92%).

### Synthesis of Intermediate 6

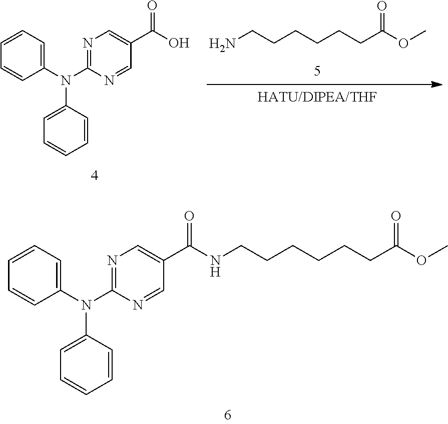 [SMILES: O=C(O)c1cnc(N(c2ccccc2)c2ccccc2)nc1 ; COC(=O)CCCCCCN ; COC(=O)CCCCCCNC(=O)c1cnc(N(c2ccccc2)c2ccccc2)nc1]

A mixture of compound 4 (2.5 g, 8.58 mmol), aminoheptanoate 5 (2.52 g, 12.87 mmol), HATU (3.91 g, 10.30 mmol), DIPEA (4.43 g, 34.32 mmol) was stirred at rt overnight. After the reaction mixture was filtered, the filtrate was evaporated to dryness and the residue was purified by silica gel chromatography (petroleum ethers/EtOAc=2/1) to give a brown solid (2 g, 54%).

### Synthesis of 2-(diphenylamino)-N-(7-(hydroxyamino)-7-oxoheptyl)pyrimidine-5-carboxamide

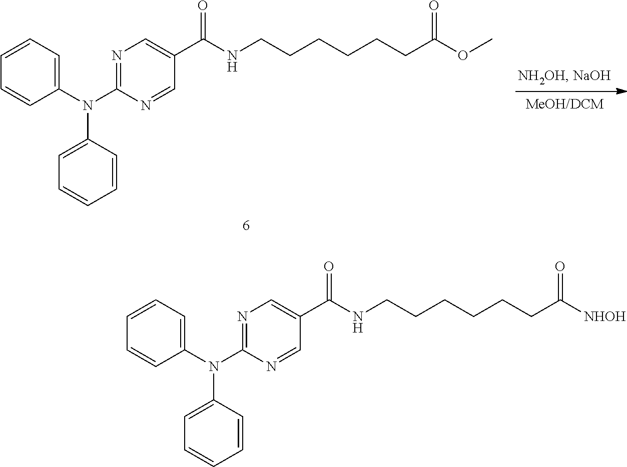

A mixture of the compound 6 (2.0 g, 4.6 mmol), sodium hydroxide (2N, 20 mL) in MeOH (50 ml) and DCM (25 ml) was stirred at 0° C. for 10 min. Hydroxylamine (50%) (10 ml) was cooled to 0° C. and added to the mixture. The resulting mixture was stirred at rt for 20 min. After removal of the solvent, the mixture was neutralized with 1M HCl to give a white precipitate. The crude product was filtered and purified by pre-HPLC to give a white solid (950 mg, 48%).

### Example 6

### Synthesis of 2-((1-(3-fluorophenyl)cyclohexyl)amino)-N-hydroxypyrimidine-5-carboxamide (Compound B)

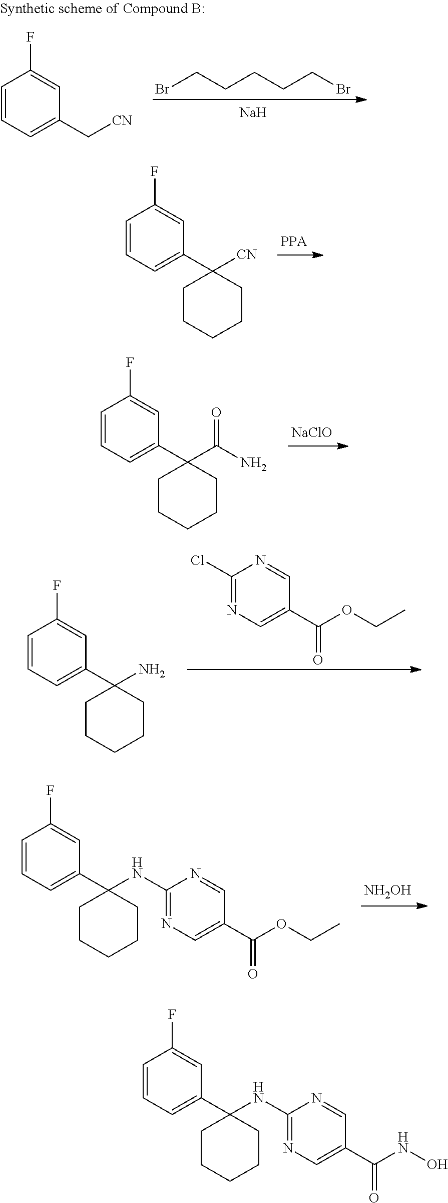

### Synthesis of 1-(3-fluorophenyl)cyclohexanecarbonitrile

To a solution of 2-(3-fluorophenyl)acetonitrile (100 g, 0.74 mol) in Dry DMF (1000 ml) was added 1,5-dibromopentane (170 g, 0.74 mol), and NaH (65 g, 2.2 eq) was added dropwise at ice bath. After addition, the resulting mixture was vigorously stirred overnight at 50° C. The suspension was quenched by ice water carefully, and extracted with ethyl acetate (3*500 ml). The combined organic solution was concentrated to afford the crude, which was purified on a flash column, to give 1-(3-fluorophenyl)cyclohexanecarbonitrile as a pale solid (100 g, 67%).

### Synthesis of 1-(3-fluorophenyl)cyclohexanecarboxamide

A solution of 1-(3-fluorophenyl)cyclohexanecarbonitrile (100 g, 0.49 mol) in PPA (500 ml) was heated at 110° C. for about 5-6 hours. After completion, the resulting mixture was carefully basified with sat.NaHCO3 soultion until pH=8-9. The precipitate was collected and washed with water (1000 ml) to afford 1-(3-fluorophenyl)cyclohexanecarboxamide as a white solid (95 g, 87%).

### Synthesis of 1-(3-fluorophenyl)cyclohexanamine

To a solution of 1-(3-fluorophenyl)cyclohexanecarboxamide (95 g, 0.43 mol) in n-BuOH (800 ml) was added NaClO (260 ml, 1.4 eq), then 3N NaOH (400 ml, 2.8 eq) was added at 0° C., and the reaction was stirred overnight at r.t. The resulting mixture was extracted with EA (2*500 ml), and the combined organic solution was washed with brine, and then dried to afford the crude, which was further purified on treating with HCl salt, as a white powder (72 g, 73%).

### Synthesis of ethyl 2-(1-(3-fluorophenyl)cyclohexylamino)pyrimidine-5-carboxylate

To a solution of 1-(3-fluorophenyl)cyclohexanamine hydrochloride (2.29 g 10 mmol) in Dioxane (50 ml) was added ethyl 2-chloropyrimidine-5-carboxylate (1.87 g, 1.0 eq) and DIPEA (2.58 g, 2.0 eq). The mixture was heated overnight at 110-120° C. The resulting mixture was directly purified on a silica gel column to afford the coupled product as a white solid (1.37 g, 40%).

### Synthesis of Compound B

To a solution of ethyl 2-(1-(3-fluorophenyl)cyclohexylamino)pyrimidine-5-carboxylate (100 mg, 0.29 mmol) in MeOH/DCM (10 ml, 1:1) was added 50% NH₂OH in water (2 ml, excess), then sat. NaOH in MeOH (2 ml, excess) was added at 0° C., and the reaction was stirred for 3-4 hours. After completion, the resulting mixture was concentrated, and acidified with 2N HCl to pH=4-5. The precipitate was collected, washed with water (10 ml) to remove the NH₂OH, and dried to afford Compound B as a white powder (70 mg, 73%).

LENGTHY TABLES
The patent application contains a lengthy table section. A copy of the table is available in electronic form from the USPTO web site (http://seqdata.uspto.gov/?pageRequest=docDetail&DocID=US20140357512A1). An electronic copy of the table will also be available from the USPTO upon request and payment of the fee set forth in 37 CFR 1.19(b)(3).

What is claimed is:

1. A Compound B specific histone deacetylase 6 (HDAC6) biomarker peptide, which is acetylated, for multiple myeloma comprising an amino acid sequence selected from the group consisting of SEQ ID NOs: 1-18, and 323-490.

2. A Tubastatin A specific histone deacetylase 6 (HDAC6) biomarker peptide, which is acetylated, for multiple myeloma comprising an amino acid sequence selected from the group consisting of SEQ ID NOs: 1-322.

3. A joint Compound B and Tubastatin A specific histone deacetylase 6 (HDAC6) biomarker peptide, which is acetylated, for multiple myeloma comprising an amino acid sequence selected from the group consisting of SEQ ID NOs: 1-18.

4. A Compound A specific histone deacetylase (HDAC) biomarker peptide, which is acetylated, for multiple myeloma comprising an amino acid sequence selected from the group consisting of SEQ ID NOs: 12-18, 245-322, and 476-817.

5. A Compound B specific histone deacetylase 6 (HDAC6) biomarker peptide, which is acetylated or unacetylated, for multiple myeloma comprising an amino acid sequence selected from the group consisting of SEQ ID NOs: 818-15721.

6. A kit comprising an anti-acetylated lysine antibody and instructions for identifying an acetylated biomarker peptide comprising an amino acid sequence selected from the group consisting of SEQ ID NOs: 1-817.

7. A kit comprising a detection agent and instructions for identifying an acetylated or unacetylated biomarker peptide comprising an amino acid sequence selected from the group consisting of SEQ ID NOs: 818-15721.

8. A method for monitoring the treatment efficiency of a histone deacetylase (HDAC) inhibitor in a subject comprising:
a) administering a therapeutically effective amount of an HDAC inhibitor to a subject;
b) taking a biological sample from the subject;
c) determining whether an HDAC biomarker peptide comprising an amino acid sequence selected from the group consisting of SEQ ID NOs: 1-15721 is present in the sample; and
d) concluding that the HDAC treatment is efficient if an HDAC biomarker peptide comprising an amino acid sequence selected from the group consisting of SEQ ID NOs: 1-15721 is present in the sample, and concluding that the HDAC treatment is not efficient if an HDAC biomarker peptide comprising an amino acid sequence selected from the group consisting of SEQ ID NOs: 1-15721 is not present in the sample.

9. The method of claim 8, wherein the HDAC inhibitor is selected from the group consisting of: Compound A, Compound B, and Tubastatin A.

10. The method of claim 8, wherein the HDAC inhibitor is an HDAC6 inhibitor.

11. The method of claim 10, wherein the HDAC6 inhibitor is selected from the group consisting of Compound B, and Tubastatin A.

12. The method of claim 8, wherein the sample is a bone marrow sample.

13. The method of claim 8, wherein the determining step utilizes an antibody.
